(19) United States
(12) Patent Application Publication
(10) Pub. No.: US 2007/0032508 A1
(43) Pub. Date: Feb. 8, 2007

(54) Quinazoline derivatives as tyrosine kinase inhibitors

(76) Inventors: Robert Hugh Bradbury, Macclesfield (GB); Jason Grant Kettle, Macclesfield (GB); Laurent Francois Andre Hennequin, Reim (FR); Bernard Christophe Barlaam, Reims (FR)

Correspondence Address: MORGAN LEWIS & BOCKIUS LLP, 1111 PENNSYLVANIA AVENUE NW, WASHINGTON, DC, 20004, US

(21) Appl. No.: 10/571,851

(22) PCT Filed: Sep. 14, 2004
(86) PCT No.: PCT/GB04/03931

§ 371 Date: Mar. 15, 2006

(30) Foreign Application Priority Data
Sep. 16, 2003 (EP) 03292279.1

(51) Int. Cl. A61K 31/517; C07D 403/02; C07D 417/02; C07D 413/02

(52) U.S. Cl. 514/255.05; 514/266.2; 514/266.21; 544/284

(57) ABSTRACT

A quinazoline derivative of the formula (I); wherein the substituents are as defined in the text for use in the production of an anti proliferative effect which effect is produced alone or in part by inhibiting erbB2 receptor tyrosine kinase in a warm blooded animal such as man.

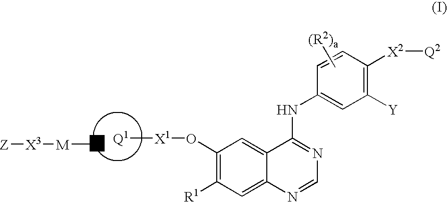

The invention concerns certain novel quinazoline derivatives, or pharmaceutically acceptable salts thereof, which possess anti-tumour activity and are accordingly useful in methods of treatment of the human or animal body. The invention also concerns processes for the manufacture of said quinazoline derivatives, to pharmaceutical compositions containing them and to their use in therapeutic methods, for example in the manufacture of medicaments for use in the prevention or treatment of solid tumour disease in a warm-blooded animal such as man.

Many of the current treatment regimes for diseases resulting from the abnormal regulation of cellular proliferation such as psoriasis and cancer, utilise compounds that inhibit DNA synthesis and cellular proliferation. To date, compounds used in such treatments are generally toxic to cells however their enhanced effects on rapidly dividing cells such as tumour cells can be beneficial. Alternative approaches to these cytotoxic anti-tumour agents are currently being developed, for example selective inhibitors of cell signalling pathways. These types of inhibitors are likely to have the potential to display an enhanced selectivity of action against tumour cells and so are likely to reduce the probability of the therapy possessing unwanted side effects.

Eukaryotic cells are continually responding to many diverse extracellular signals that enable communication between cells within an organism. These signals regulate a wide variety of physical responses in the cell including proliferation, differentiation, apoptosis and motility. The extracellular signals take the form of a diverse variety of soluble factors including growth factors as well as paracrine and endocrine factors. By binding to specific transmembrane receptors, these ligands integrate the extracellular signal to the intracellular signalling pathways, therefore transducing the signal across the plasma membrane and allowing the individual cell to respond to its extracellular signals. Many of these signal transduction processes utilise the reversible process of the phosphorylation of proteins that are involved in the promotion of these diverse cellular responses. The phosphorylation status of target proteins is regulated by specific kinases and phosphatases that are responsible for the regulation of about one third of all proteins encoded by the mammalian genome. As phosphorylation is such an important regulatory mechanism in the signal transduction process, it is therefore not surprising that aberrations in these intracellular pathways result in abnormal cell growth and differentiation and so promote cellular transformation (reviewed in Cohen et al, Curr Opin Chem Biol 1999, 3, 459-465).

It has been widely shown that a number of these tyrosine kinases are mutated to constitutively active forms and/or when over-expressed result in the transformation of a variety of human cells. These mutated and over-expressed forms of the kinase are present in a large proportion of human tumours (reviewed in Kolibaba et al, Biochimica et Biophysica Acta, 1997, 133, F217-F248). As tyrosine kinases play fundamental roles in the proliferation and differentiation of a variety of tissues, much focus has centred on these enzymes in the development of novel anti-cancer therapies. This family of enzymes is divided into two groups—receptor and non-receptor tyrosine kinases e.g. EGF Receptors and the SRC family respectively. From the results of a large number of studies including the Human Genome Project, about 90 tyrosine kinase have been identified in the human genome, of this 58 are of the receptor type and 32 are of the non-receptor type. These can be compartmentalised in to 20 receptor tyrosine kinase and 10 non-receptor tyrosine kinase sub-families (Robinson et al, Oncogene, 2000, 19, 5548-5557).

The receptor tyrosine kinases are of particular importance in the transmission of mitogenic signals that initiate cellular replication. These large glycoproteins, which span the plasma membrane of the cell possess an extracellular binding domain for their specific ligands (such as Epidermal Growth Factor (EGF) for the EGF Receptor). Binding of ligand results in the activation of the receptor's kinase enzymatic activity that is encoded by the intracellular portion of the receptor. This activity phosphorylates key tyrosine amino acids in target proteins, resulting in the transduction of proliferative signals across the plasma membrane of the cell.

It is known that the erbB family of receptor tyrosine kinases, which include EGFR, erbB2, erbB3 and erbB4, are frequently involved in driving the proliferation and survival of tumour cells (reviewed in Olayioye et al., EMBO J., 2000, 19, 3159). One mechanism in which this can be accomplished is by overexpression of the receptor at the protein level, generally as a result of gene amplification. This has been observed in many common human cancers (reviewed in Klapper et al., Adv. Cancer Res., 2000, 77, 25) such as breast cancer (Sainsbury et al., Brit. J. Cancer, 1988, 58, 458; Guerin et al., Oncogene Res., 1988, 3, 21; Slamon et al., Science, 1989, 244, 707; Klijn et al., Breast Cancer Res. Treat., 1994, 29, 73 and reviewed in Salomon et al., Crit. Rev. Oncol. Hematol. 1995, 19, 183), non-small cell lung cancers (NSCLCs) including adenocarcinomas (Cerny et al., Brit. J. Cancer, 1986, 54, 265; Reubi et al, Int. J. Cancer 1990, 45, 269; Rusch et al., Cancer Research, 1993, 53, 2379; Brabender et al, Clin. Cancer Res., 2001, 7, 1850) as well as other cancers of the lung (Hendler et al., Cancer Cells, 1989, 7, 347; Ohsaki et al. Oncol. Rep., 2000, 7, 603), bladder cancer (Neal et al., Lancet, 1985, 366; Chow et al., Clin. Cancer Res., 2001, 7, 1957, Zhau et al, Mol Carcinog. 3, 254), oesophageal cancer (Nukaida et al., Cancer, 1991, 68, 142), gastrointestinal cancer such as colon, rectal or stomach cancer (Bolen et al., Oncogene Res. 1987, 1, 149; Kapitanovic et al., Gastroenterology, 2000, 112, 1103; Ross et al. Cancer Invest., 2001, 19, 554), cancer of the prostate (Visakorpi et al., Histochem. J., 1992, 24, 481; Kumar et al. 2000, 32, 73; Scher et al. J. Natl. Cancer Inst., 2000, 92, 1866), leukaeniia (Konaka et al., Cell 1984, 37, 1035, Martin-Subero et al., Cancer Genet Cytogenet., 2001, 127, 174), ovarian (Hellstrom et al., Cancer Res., 2001, 61, 2420), head and neck (Shiga et al., Head Neck, 2000, 22, 599) or pancreatic cancer (Ovotny et al., Neoplasma, 2001, 48, 188). As more human tumour tissues are tested for expression of the erbB family of receptor tyrosine kinases it is expected that their widespread prevalence and importance will be further enhanced in the future.

As a consequence of the mis-regulation of one or more of these receptors (in particular erbB2), it is widely believed that many tumours become clinically more aggressive and so correlate with a poorer prognosis for the patient (Brabender et al., Clin. Cancer Res., 2001, 7, 1850; Ross et al Cancer Investigation, 2001, 19, 554, Yu et al., Bioessays. 2000, 22.7, 673). In addition to these clinical findings, a wealth of pre-clinical information suggests that the erbB family of receptor tyrosine kinases are involved in cellular transformation. This includes the observations that many tumour cell lines overexpress one or more of the erbB receptors and that EGFR or erbB2 when transfected into non-tumour cells have the ability to transform these cells. This tumourigenic potential has been further verified as transgenic mice that overexpress erbB2 spontaneously develop tumours in the mammary gland. In addition to this, a number of pre-clinical studies have demonstrated that anti-proliferative effects can be induced by knocking out one or more erbB activities by small molecule inhibitors, dominant negatives or inhibitory antibodies (reviewed in Mendelsohn et al., Oncogene, 2000, 19, 6550). Thus it has been recognised that inhibitors of these receptor tyrosine kinases should be of value as a selective inhibitor of the proliferation of mammalian cancer cells (Yaish et al. Science, 1988, 242, 933, Kolibaba et al, Biochimica et Biophysica Acta, 1997, 133, F217-F248; Al-Obeidi et al, 2000, Oncogene, 19, 5690-5701; Mendelsohn et al, 2000, Oncogene, 19, 6550-6565). In addition to this pre-clinical data, findings using inhibitory antibodies against EGFR and erbB2 (c-225 and trastuzumab respectively) have proven to be beneficial in the clinic for the treatment of selected solid tumours (reviewed in Mendelsohn et al, 2000, Oncogene 19, 6550-6565).

Amplification and/or activity of members of the ErbB type receptor tyrosine kinases have been detected and so have been implicated to play a role in a number of non-malignant proliferative disorders such as psoriasis (Ben-Bassat, Curr. Pharm. Des., 2000, 6, 933; Elder et al., Science, 1989, 243, 811), benign prostatic hyperplasia (BPH) (Kumar et al., Int. Urol. Nephrol., 2000, 32, 73), atherosclerosis and restenosis (Bokemeyer et al., Kidney Int. 2000, 58, 549). It is therefore expected that inhibitors of erbB type receptor tyrosine kinases will be useful in the treatment of these and other non-malignant disorders of excessive cellular proliferation.

International Patent Applications WO 96/09294, WO 96/15118, WO 96/16960, WO 96/30347, WO 96/33977, WO 96/33978, WO 96/33979, WO 96/33980, WO 96/33981, WO 97/03069, WO 97/13771, WO 97/30034, WO 97/30035, WO 97/38983, WO 98/02437, WO 98/02434, WO 98/02438, WO 98/13354, WO 99/35132, WO 99/35146, WO01/21596, WO01/21594, WO 01/55141 and WO 02/18372 disclose that certain quinazoline derivatives which bear an anilino substituent at the 4-position possess receptor tyrosine kinase inhibitory activity.

International Patent Applications WO01/94341 discloses that certain quinazoline derivatives which carry a 5-substituent are inhibitors of the Src family of non-receptor tyrosine kinases, such as c-Src, c-Yes and c-Fyn.

International Patent applications WO03/040108 and WO03/040109 disclose that certain quinazoline derivatives which carry a 5-substituent are inhibitors of the erbB family of tyrosine kinase inhibitors, particularly EGFR and erb-B2 receptor tyrosine kinases. International Patent Application WO2004/006846 discloses that certain quinazoline derivatives which carry substituents at the 4-, 6- and 7-positions modulate ephrin and EGFR receptor kinase activity. A specific example of such a compound is 7-[(cyclopropylmethyl)oxy]-N-(3,4-dichlorophenyl)-6-(methyloxy)quinazolin-4-amine.

We have now found that surprisingly certain quinazoline derivatives substituted at the 6-position with a substituent containing certain alkanoyl or sulfonyl groups (more specifically substituted at the 6-position with a substituent containing a 4, 5, 6 or 7 membered saturated or partially unsaturated heterocyclyl group containing 1 nitrogen heteroatom and optionally 1 or 2 additional heteroatoms selected from O, S and N, which heterocyclyl group is substituted on the nitrogen heteroatom by certain alkanoyl or sulfonyl groups) possess potent anti-tumour activity. Without wishing to imply that the compounds disclosed in the present invention possess pharmacological activity only by virtue of an effect on a single biological process, it is believed that the compounds provide an anti-tumour effect by way of inhibition of one or more of the erbB family of receptor tyrosine kinases that are involved in the signal transduction steps which lead to the proliferation of tumour cells. In particular, it is believed that the compounds of the present invention provide an anti-tumour effect by way of inhibition of EGFR and/or erbB2 (particularly erbB2) receptor tyrosine kinases.

Generally the compounds of the present invention possess potent inhibitory activity against the erbB receptor tyrosine kinase family, for example by inhibition of EGFR and/or erbB2 and/or erbB4 receptor tyrosine kinases, whilst possessing less potent inhibitory activity against other kinases. Furthermore, generally the compounds of the present invention possess substantially better potency against the erbB2 over that of the EGFR tyrosine kinase, thus potentially providing effective treatment for erbB2 driven tumours. Accordingly, it may be possible to administer a compound according to the present invention at a dose that is sufficient to inhibit erbB2 tyrosine kinase whilst having no significant effect upon EGFR (or other) tyrosine kinases. The selective inhibition provided by the compounds according to the present invention may provide treatments for conditions mediated by erbB2 tyrosine kinase, whilst reducing undesirable side effects that may be associated with the inhibition of other tyrosine kinases.

Generally the compounds according to the invention exhibit favourable DMPK properties, for example high bioavailability and/or high free-plasma levels.

According to a first aspect of the invention there is provided a quinazoline derivative of the Formula I: 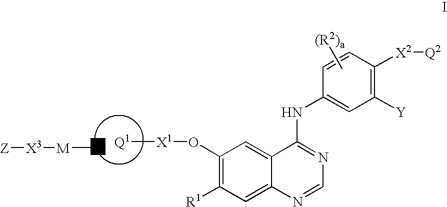
wherein:

R¹ is selected from hydrogen, hydroxy, (1-6C)alkoxy, (3-7C)cycloalkyl-oxy and (3-7C)cycloalkyl-(1-6C)alkoxy,

and wherein adjacent carbon atoms in any (2-6C)alkylene chain within a R¹ substituent are optionally separated by the insertion into the chain of a group selected from O, S, SO, SO₂, N(R³), CO, CON(R³), N(R³)CO, SO₂N(R³) and N(R³)SO₂, wherein R³ is hydrogen or (1-6C)alkyl,

and wherein any CH₂ or CH₃ group within a R¹ substituent optionally bears on each said CH₂ or CH₃ group one or more halogeno or (1-6C)alkyl substituents, or a substituent selected from hydroxy, cyano, amino, carboxy, carbamoyl, sulfamoyl, oxo, thioxo, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino;

Y is selected from hydrogen, halogeno, (1-4C)alkyl, (1-4C)alkoxy, (2-4C)alkenyl and (2-4C)alkynyl;

a is 0, 1, 2 or 3 or 4;

each R², which may be the same or different, is selected from halogeno, (1-4C)alkyl, (1-4C)alkoxy, (2-4C)alkenyl and (2-4C)alkynyl;

X² is a direct bond or is selected from O, S, OC(R⁴)₂, SC(R⁴)₂, SO, SO₂, N(R⁴), CO and N(R⁴)C(R⁴)₂ wherein each R⁴ is, which may be the same or different, is selected from hydrogen or (1-6C)alkyl, and Q² is aryl or heteroaryl,

and wherein Q² optionally bears one or more substituents (for example 1, 2 or 3), which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, and a group of the formula:
—X⁴—R⁵

wherein X⁴ is a direct bond or is selected from O, CO and N(R⁶), wherein R⁶ is hydrogen or (1-6C)alkyl, and R⁵ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulfamoyl(1-6C)alkyl, N-(1-6C)alkylsulfamoyl(1-6C)alkyl, N,N di-(1-6C)alkylsulfamoyl(1-6C)alkyl, (2-6C)alkanoyl-(1-6C)alkyl, (2-6C)alkanoyloxy-(1-6C)alkyl or (1-6C)alkoxycarbonyl-(1-6C)alkyl,

and wherein any CH₂ or CH₃ group within-X²-Q² optionally bears on each said CH₂ or CH₃ one or more (for example 1, 2, or 3) halogeno or (1-6C)alkyl substituents or a substituent selected from hydroxy, cyano, amino, (1-4C)alkoxy, (1-4C)alkylamino and di-[(1-4C)alkylamino];

X¹ is a direct bond or C(R⁷)₂, wherein each R⁷, which may be the same or different, is selected from hydrogen and (1-4C)alkyl;

ring Q¹ is a 4, 5, 6 or 7 membered saturated or partially unsaturated heterocyclyl group containing 1 nitrogen heteroatom and optionally 1 or 2 additional heteroatoms selected from O, S and N, and which ring is linked to the group X¹ by a ring carbon;

M is selected from CO and SO₂;

X³ is a group of the formula:
—(CR⁸R⁹)ₚ-(Q³)ₘ-(CR¹⁰R¹¹)_{q}—

wherein m is 0 or 1, p is 0, 1, 2, 3 or 4 and q is 0, 1, 2, 3 or 4,

each of R⁸, R⁹, R¹⁰ and R¹¹, which may be the same or different, is selected from hydrogen and (1-6C)alkyl, and

Q³ is selected from (3-7C)cycloalkylene and (3-7C)cycloalkenylene;

Z is selected from hydrogen, hydroxy, amino, (1-6C)alkylamino, di-[(1-6C)alkyl]amino, (1-6C)alkoxy, (1-6C)alkylsulfonyl, (1-6C)alkanesulfonylamino, N-(1-6C)alkyl-(1-6C)alkanesulfonylamino, and a group of the formula:
Q⁴-X⁵—

wherein X⁵ is a direct bond or is selected from O, N(R¹²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein adjacent carbon atoms in any (2-6C)alkylene chain within a Z substituent are optionally separated by the insertion into the chain of a group selected from O, S, SO, SO₂, N(R¹³), CO, —C═C— and —C≡C— wherein R¹³ is hydrogen or (1-6C)alkyl,

and wherein any CH₂ or CH₃ group within any Z, X¹ or X³ group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group represented by Q¹ or within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group represented by Q¹ or within a Z substituent optionally bears 1 or 2 oxo or thioxo substituents;

or a pharmaceutically acceptable salt thereof.

According to a second aspect of the invention there is provided a quinazoline derivative of the Formula I, wherein R¹ is selected from hydrogen, hydroxy and (1-6C)alkoxy,

and wherein adjacent carbon atoms in any (2-6C)alkylene chain within a R¹ substituent are optionally separated by the insertion into the chain of a group selected from O, S, SO, SO₂, N(R³), CO, CON(R³), N(R³)CO, SO₂N(R³) and N(R³)SO₂, wherein R³ is hydrogen or (1-6C)alkyl,

and wherein any CH₂ or CH₃ group within a R¹ substituent optionally bears on each said CH₂ or CH₃ group one or more halogeno or (1-6C)alkyl substituents, or a substituent selected from hydroxy, cyano, amino, carboxy, carbamoyl, sulfamoyl, oxo, thioxo, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino;

and wherein Y, a, R², X², Q², X¹, ring Q¹, M, X³ and Z are each as hereinbefore defined,

or a pharmaceutically acceptable salt thereof.

In particular, in the quinazoline derivatives of the Formula I defined above, when X² is CO or SO, then M is not CO.

In this specification the generic term “alkyl” includes both straight-chain and branched-chain alkyl groups such as propyl, isopropyl and tert-butyl, and (3-7C)cycloalkyl groups such as cyclopropyl, cyclobutyl cyclopentyl, cyclohexyl and cycloheptyl. However references to individual alkyl groups such as “propyl” are specific for the straight-chain version only, references to individual branched-chain alkyl groups such as “isopropyl” are specific for the branched-chain version only and references to individual cycloalkyl groups such as “cyclopentyl” are specific for that 5-membered ring only. An analogous convention applies to other generic terms, for example (1-6C)alkoxy includes methoxy, ethoxy, cyclopropyloxy and cyclopentyloxy, (1-6C)alkylamino includes methylamino, ethylamino, cyclobutylamino and cyclohexylamino, and di-[(1-6C)alkyl]amino includes dimethylamino, diethylamino, N-cyclobutyl-N-methylamino and N-cyclohexyl-N-ethylamino.

It is to be understood that, insofar as certain of the compounds of Formula I defined above may exist in optically active or racemic forms by virtue of one or more asymmetric carbon atoms, the invention includes in its definition any such optically active or racemic form which possesses the above-mentioned activity. It is further to be understood that in the names of chiral compounds (R,S) denotes any scalemic or racemic mixture while (R) and (S) denote the enantiomers. In the absence of (R,S), (R) or (S) in the name it is to be understood that the name refers to any scalemic or racemic mixture, wherein a scalemic mixture contains R and S enantiomers in any relative proportions and a racemic mixture contains R and S enantiomers in the ratio 50:50. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

Suitable values for the generic radicals referred to above include those set out below.

A suitable value for any one of the ‘Q’ groups (for example Q²) when it is aryl or for the aryl group within a ‘Q’ group is, for example, phenyl or naphthyl, preferably phenyl. A suitable value for any one of the ‘Q’ groups (for example Q⁴) when it is (3-7C)cycloalkyl or for the (3-7C)cycloalkyl group within a ‘Q’ group or a R¹ group is, for example, cyclopropyl, cyclobutyl, cyclopentyl, cyclohexyl, cycloheptyl or bicyclo[2.2.1]heptyl and a suitable value for any one of the ‘Q’ groups (for example Q¹) when it is (3-7C)cycloalkenyl or for the (3-7C)cycloalkenyl group within a ‘Q’ group is, for example, cyclobutenyl, cyclopentenyl cyclohexenyl or cycloheptenyl. It is to be understood that reference to (3-7C)cycloalkylene used herein for Q³ refers to a divalent (3-7C)cycloalkane linking group, which group may be linked via different carbon atoms in the (3-7C)cycloalkylene ring, or which may be linked via a single carbon atom in the (3-7C)cycloalkylene ring. Accordingly, reference to, for example, a “cyclopropylene” group includes cycloprop-1,2-ylene and a cyclopropylidene group of the formula: 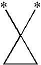
wherein * represent the bonds from the divalent cyclopropylidene group.

However references to an individual (3-7C)cycloalkylene group such as cyclopropylidene are specific for that group only. A similar convention is adopted for the (3-7C)cycloalkenylene groups represented by Q³.

References to (3-7C)cycloalkyl-oxy groups include, for example, cyclopropyl-oxy, cyclobutyl-oxy, cyclopentyl-oxy, cyclohexyl-oxy, cycloheptyl-oxy or bicyclo[2.2.1]heptyloxy. References to (3-7C)cycloalkyl-(1-6C)alkoxy groups include, for example, cyclopropyl-(1-6C)alkoxy, cyclobutyl-(1-6C)alkoxy, cyclopentyl-(1-6C)alkoxy, cyclohexyl-(1-6C)alkoxy, cycloheptyl-(1-6C)alkoxy or bicyclo[2.2.1]heptyl-(1-6C)alkoxy, where the (1-6C)alkoxy group may be, for example, methoxy, ethoxy, propoxy, isopropoxy or butoxy. Particular values for (3-7C)cycloalkyl-(1-6C)alkoxy groups include, for example, cyclopropylmethoxy and cyclopropylethoxy.

A suitable value for any one of the ‘Q’ groups (for example Q²) when it is heteroaryl or for the heteroaryl group within a ‘Q’ group is, for example, an aromatic 5- or 6-membered monocyclic ring or a 9- or 10-membered bicyclic ring with up to five ring heteroatoms independently selected from oxygen, nitrogen and sulfur, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, 1,3-benzodioxolyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl or naphthyridinyl.

A suitable value for any one of the ‘Q’ groups (for example Q¹ or Q⁴) when it is heterocyclyl or for the heterocyclyl group within a ‘Q’ group is, for example, a non-aromatic saturated (i.e. ring systems with the maximum degree of saturation) or partially saturated (i.e. ring systems retaining some, but not the full, degree of unsaturation) 3 to 10 membered monocyclic or bicyclic ring with up to five heteroatoms independently selected from oxygen, nitrogen and sulfur, which, unless specified otherwise, may be carbon or nitrogen linked, for example oxiranyl, oxetanyl, azetidinyl, tetrahydrofuranyl, 1,3-dioxolanyl, tetrahydropyranyl, 1,4-dioxanyl, oxepanyl, pyrrolinyl, pyrrolidinyl, morpholinyl, tetrahydro-1,4-thiazinyl, 1,1-dioxotetrahydro-1,4-thiazinyl, piperidinyl, homopiperidinyl, piperazinyl, homopiperazinyl, dihydropyridinyl, tetrahydropyridinyl, dihydropyrimidinyl, tetrahydropyrimidinyl, tetrahydrothienyl, tetrahydrothiopyranyl, decahydroisoquinolinyl or decahydroquinolinyl, particularly tetrahydrofuanyl, tetrahydropyranyl, pyrrolidinyl, morpholinyl, 1,4-oxazepanyl, thiamorpholinyl 1,1-dioxotetrahydro-4H-1,4-thiazinyl, piperidinyl or piperazinyl, more particularly tetrahydrofuran-3-yl, tetrahydropyranyl, tetrahydrothien-3-yl, tetrahydrothiopyran-4-yl, pyrrolidin-1-yl pyrrolidin-2-yl, pyrrolidin-3-yl, morpholino, morpholin-2-yl, piperidino, piperidin-4-yl, piperidin-3-yl, piperidin-2-yl or piperazin-1-yl. A nitrogen or sulfur atom within a heterocyclyl group may be oxidized to give the corresponding N or S oxide, for example 1,1-dioxotetrahydrothienyl, 1-oxotetrahydrothienyl, 1,1-dioxotetrahydrothiopyranyl or 1-oxotetrahydrothiopyranyl. A suitable value for such a group which bears 1 or 2 oxo or thioxo substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl.

A suitable value for any one of the ‘Q’ groups (for example Q¹) when it is a nitrogen containing heterocyclyl group is, for example, a non-aromatic saturated or partially saturated 3 to 10 membered monocyclic or bicyclic ring with up to five heteroatoms independently selected from oxygen, nitrogen and sulfur, provided at least one heteroatom is nitrogen, which, unless specified otherwise, may be carbon or nitrogen linked. Suitable values include, for example, those heterocyclic groups mentioned above that contain at least one nitrogen atom, for example azetidinyl, pyrrolinyl, pyrrolidinyl, morpholinyl (including morpholino), tetrahydro-1,4-thiazinyl, 1,1-dioxotetrahydro-1,4-thiazinyl, piperidinyl (including piperidino), homopiperidinyl, piperazinyl, homopiperazinyl, dihydropyridinyl, tetrahydropyridinyl, dihydropyrimidinyl, tetrahydropyrimidinyl, decahydroisoquinolinyl or decahydroquinolinyl.

Particular values for Q¹ is a carbon linked 4, 5, 6 or 7 membered monocyclic heterocyclyl group containing 1 nitrogen heteroatom and optionally 1 or 2 further heteroatoms independently selected from oxygen, nitrogen and sulfur, which heterocyclyl group may be fully saturated or partially saturated. More particularly Q¹ is a carbon linked 5 or 6 membered monocyclic heterocyclyl group containing 1 nitrogen heteroatom and optionally 1 further heteroatom selected from oxygen, nitrogen and sulfur, which heterocyclyl group may be partially saturated or preferably fully saturated. Still more particularly Q¹ is a carbon linked monocyclic fully saturated 5 or 6 membered monocyclic heterocyclyl group containing 1 nitrogen heteroatom and optionally 1 further heteroatom selected from oxygen, nitrogen and sulfur. Suitable values of such groups represented by Q¹ include the appropriate heterocyclyl groups listed above, more particularly azetidinyl, pyrrolidinyl, piperidinyl, piperazinyl or morpholinyl (all of which are linked to X¹ by a ring carbon), more particularly, pyrrolidin-2-yl pyrrolidin-3-yl, piperidin-4-yl, piperidin-3-yl, piperidin-2-yl, piperazin-2-yl, morpholin-2-yl or morpholin-3-yl, and still more particularly pyrrolidin-2-yl, pyrrolidin-3-yl, piperidin-3-yl, piperidin-2-yl, piperazin-2-yl, morpholin-2-yl or morpholin-3-yl.

For the avoidance of any doubt the nitrogen atom in Q¹ to which the group ZX³M is attached is not quaternised; namely the group ZX³M is attached to the nitrogen atom in Q¹ via substitution of an NH group in the heterocyclyl ring, for example when Q¹ is pyrrolidin-2-yl the ZX³M group is attached to the pyrrolidin-2-yl ring at the 1-position.

A suitable value for a ‘Q’ group when it is heterocyclyl-(1-6C)alkyl is, for example, heterocyclylmethyl, 2-heterocyclylethyl and 3-heterocyclylpropyl. The invention comprises corresponding suitable values for ‘Q’ groups when, for example, rather than a heterocyclyl-(1-6C)alkyl group, an (3-7C)cycloalkyl-(1-6C)alkyl or (3-7C)cycloalkenyl-(1-6C)alkyl is present.

Suitable values for any of the ‘R’ groups (R¹ to R¹⁵), Y, or for various groups within a Q¹, Q², X³ or Z group include:—
for halogeno fluoro, chloro, bromo and iodo;
for (1-6C)alkyl: methyl, ethyl, propyl, isopropyl and tert-butyl;
for (2-8C)alkenyl: vinyl, isopropenyl, allyl and but-2-enyl;
for (2-8C)alkynyl: ethynyl, 2-propynyl and but-2-ynyl;
for (1-6C)alkoxy: methoxy, ethoxy, propoxy, isopropoxy and butoxy;
for (2-6C)alkenyloxy: vinyloxy and allyloxy;
for (2-6C)alkynyloxy: ethynyloxy and 2-propynyloxy;
for (1-6C)alkylthio: methylthio, ethylthio and propylthio;
for (1-6C)alkylsulfinyl: methylsulfinyl and ethylsulfinyl;
for (1-6C)alkylsulfonyl: methylsulfonyl and ethylsulfonyl;
for (1-6C)alkylamino: methylamino, ethylamino, propylamino, isopropylamino and butylamino;
for di-[(1-6C)alkyl]amino: dimethylamino, diethylamino, N-ethyl-N-methylamino and diisopropylamino;
for (1-6C)alkoxycarbonyl: methoxycarbonyl, ethoxycarbonyl, propoxycarbonyl and tert-butoxycarbonyl;
for N-(1-6C)alkylcarbamoyl: N-methylcarbamoyl, N-ethylcarbamoyl and N-propylcarbamoyl;
for N,N-di-[(1-6C)alkyl]carbamoyl: N,N-dimethylcarbamoyl, N-ethyl-N-methylcarbamoyl and N,N-diethylcarbamoyl;
for (2-6C)alkanoyl: acetyl, propionyl, butyryl and isobuyryl;
for (2-6C)alkanoyloxy: acetoxy and propionyloxy;
for (2-6C)alkanoylamino: acetamido and propionamido;
for N-(1-6C)alkyl-(2-6C)alkanoylamino: N-methylacetamido and N-methylpropionamido;
for N-(1-6C)alkylsulfamoyl: N-methylsulfamoyl and N-ethylsulfamoyl;
for N,N-di-[(1-6C)alkyl]sulfamoyl: N,N-sulfdimethylsulfamoyl;
for (1-6C)sulfalkanesulfonylamino: sulfmethanesulfonylamino and sulfethanesulfonylamino;
for N-(1-6C)alkyl-(1-6C)sulfalkanesulfonylamino: N-sulfmethylmethanesulfonylamino and N-sulfmethylethanesulfonylamino;
for (3-6C)alkenoylamino: acrylamido, methacrylamido and crotonamido;
for N-(1-6C)alkyl-(3-6C)alkenoylamino: N-methylacrylamido and N-methylcrotonamido;
for (3-6C)alkynoylamino: propiolamido;
for N-(1-6C)alkyl-(3-6C)alkynoylamino: N-methylpropiolamido;
for amino-(1-6C)alkyl: aminomethyl, 2-aminoethyl, 1-aminoethyl and 3-aminopropyl;
for (1-6C)alkylamino-(1-6C)alkyl: methylaminomethyl, ethylaminomethyl, 1-methylaminoethyl, 2-methylaminoethyl, 2-ethylaminoethyl and 3-methylaminopropyl;
for di-[(1-6C)alkyl]amino-(1-6C)alkyl: dimethylaminomethyl, diethylaminomethyl, 1-dimethylaminoethyl, 2-dimethylaminoethyl and 3-dimethylaminopropyl;
for halogeno-(1-6C)alkyl: chloromethyl, 2-chloroethyl, 1-chloroethyl and 3-chloropropyl;
for hydroxy-(1-6C)alkyl: hydroxymethyl, 2-hydroxyethyl, 1-hydroxyethyl and 3-hydroxypropyl;
for (1-6C)alkoxy-(1-6C)alkyl: methoxymethyl, ethoxymethyl, 1-methoxyethyl, 2-methoxyethyl, 2-ethoxyethyl and 3-methoxypropyl;
for cyano-(1-6C)alkyl: cyanomethyl, 2-cyanoethyl, 1-cyanoethyl and 3-cyanopropyl;
for (1-6C)alkylthio-(1-6C)alkyl: methylthiomethyl, ethylthiomethyl, 2-methylthioethyl, 1-methylthioethyl and 3-methylthiopropyl;
for (1-6C)alkylsulfinyl-(1-6C)alkyl: sulfmethylsulfinylmethyl, ethylsulfinylmethyl, 2-methylsulfinylethyl, 1-methylsulfinylethyl and 3-methylsulfinylpropyl;
for (1-6C)alkylsulfonyl-(1-6C)alkyl: sulfmethylsulfonylmethyl, ethylsulfonylmethyl, 2-methylsulfonylethyl, 1-methylsulfonylethyl and 3-methylsulfonylpropyl;
for (2-6C)alkanoylamino-(1-6C)alkyl: acetamidomethyl, propionamidomethyl and 2-acetamidoethyl;
for N-(1-6C)alkyl-(2-6C)alkanoylamino-(1-6C)alkyl: N-methylacetamidomethyl, 2-(N-methylacetamido)ethyl and 2-t-methylpropionamido)ethyl;
for (1-6C)alkoxycarbonylamino-(1-6C)alkyl: methoxycarbonylaminomethyl, ethoxycarbonylaminomethyl, tert-butoxycarbonylaminomethyl and 2-methoxycarbonylaminoethyl;
(2-6C)alkanoyloxy-(1-6C)alkyl: acetoxymethyl, 2-acetoxyethyl and 2-propionyloxyethyl;
for carbamoyl-(1-6C)alkyl: carbamoylmethyl, 1-carbamoylethyl, 2-carbamoylethyl and 3-carbamoylpropyl;
for (2-6C)alkanoyl-(1-6C)alkyl: acetylmethyl and 2-acetylethyl;
for N-(1-6C)alkylcarbamoyl-(1-6C)alkyl: N-methylcarbamoylmethyl, N-ethylcarbamoylmethyl, N-propylcarbamoylmethyl 1-(N-methylcarbamoyl) ethyl, 1-(N-methylcarbamoyl)ethyl, 2-(N-methylcarbamoyl)ethyl, 2-(N-ethylcarbamoyl)ethyl and 3-(N-methylcarbamoyl)propyl;
for N,N-di[(1-6C)alkyl]carbamoyl-(1-6C)alkyl: N,N-dimethylcarbamoylmethyl, N,N-diethylcarbamoylmethyl, 2-(N,N-diethylcarbamoyl)ethyl, and 3-(N,N-dimethylcarbamoyl)propyl;
for sulfamoyl(1-6C)alkyl: sulfamoylmethyl, 1-sulfamoylethyl, 2-sulfamoylethyl and 3-sulfamoylpropyl;
for N-(1-6C)alkylsulfamoyl(1-6C)alkyl: N-methylsulfamoylmethyl, N-ethylsulfamoylmethyl, N-propylsulfamoylmethyl, 1-(N-methylsulfamoyl)ethyl, 2-(N-methylsulfamoyl)ethyl and 3-N-methylsulfamoyl)propyl; and
for N,N di-(1-6C)alkylsulfamoyl(1-6C)alkyl: N,N-dimethylsulfamoylmethyl, N,N-diethylsulfamoylmethyl, N methyl-N-ethylsulfamoylmethyl, N,N-dimethylsulfamoyl)ethyl, 1-(N,N-diethylsulfamoyl)ethyl, 2-(N-dimethylsulfamoyl)ethyl, 2-(N,N-diethylsulfamoyl)ethyl and 3-(N,N-dimethylsulfamoyl)propyl.

When, as defined hereinbefore, in the group of the formula —X²-Q², and X² is, for example, a OC(R⁴)₂ linking group, it is the oxygen atom, not the carbon atom, of the OC(R⁴)₂ linking group which is attached to the phenyl ring in the Formula I and the carbon atom is attached to the Q² group. Similarly when X² is a N(R⁴)C(R⁴)₂ linking group the nitrogen atom of the N(R⁴)C(R⁴)₂ group is attached to the phenyl ring in Formula I and the carbon atom is attached to the Q² group. A similar convention is applied to other linking groups used herein, for example when Z is a group of the formula Q⁴-X⁵—, and X⁵ is SO₂N(R¹⁰), the SO₂ group is attached to Q⁴ and the nitrogen atom is attached to X³ in Formula I. Similarly, when X³ is Q³-(CR⁸R⁹)ₘ, the Q³ is attached to the group Z in Formula I and the (CR⁸R⁹)ₘ group is attached to the M group in Formula I.

It is to be understood that references herein to adjacent carbon atoms in any (2-6C)alkylene chain within a group may be optionally separated by the insertion into the chain of a group such as O or C≡C refer to insertion of the specified group between two carbon atoms in an alkylene chain. For example, when Z is a 2-pyrrolidin-1-ylethoxy group insertion of a C≡C group into the ethylene chain gives rise to a 4-pyrrolidin-1-ylbut-2-ynyloxy group.

When reference is made herein to a CH₂ or CH₃ group optionally bearing on each said CH₂ or CH₃ group one or more halogeno or (1-6C)alkyl substituents, there are suitably 1 or 2 halogeno or (1-6C)alkyl substituents present on each said CH₂ group and there are suitably 1, 2 or 3 such substituents present on each said CH₃ group.

Where reference is made herein to any CH₂ or CH₃ group optionally bearing on each said CH₂ or CH₃ group a substituent as defined herein, suitable substituents so formed include, for example, hydroxy-substituted heterocyclyl-(1-6C)alkoxy groups such as 2-hydroxy-3-piperidinopropoxy and 2-hydroxy-3-morpholinopropoxy, hydroxy-substituted heterocyclyl-(1-6C)alkylamino groups such as 2-hydroxy-3-piperidinopropylamino and 2-hydroxy-3-morpholinopropylamino, and hydroxy-substituted (2-6)alkanoyl groups such as hydroxyacetyl, 2-hydroxypropionyl and 2-hydroxybutyryl.

It is to be understood that certain compounds of the Formula I may exist in solvated as well as unsolvated forms such as, for example, hydrated forms. It is to be understood that the invention encompasses all such solvated forms which exhibit an inhibitory effect on an erbB receptor tyrosine kinase.

It is also to be understood that certain compounds of the Formula I may exhibit polymorphism, and that the invention encompasses all such forms which exhibit an inhibitory effect on an erbB receptor tyrosine kinase.

It is also to be understood that the invention relates to all tautomeric forms of the compounds of the Formula I forms which exhibit an inhibitory effect on an erbB receptor tyrosine kinase.

A suitable pharmaceutically-acceptable salt of a compound of the Formula I is, for example, an acid-addition salt of a compound of the Formula I, for example an acid-addition salt with an inorganic or organic acid such as hydrochloric, hydrobromic, sulfuric, trifluoroacetic, citric or maleic acid; or, for example, a salt of a compound of the Formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine.

Particular novel compounds of the invention include, for example, quinazoline derivatives of the Formula I, or pharmaceutically-acceptable salts thereof, wherein, unless otherwise stated, each of R¹, R², Q¹, Q², X¹, X², X³, Y, M, a and Z has any of the meanings defined hereinbefore or in paragraphs (a) to (wwwwwwww) hereinafter:—

(a) R¹ is selected from hydrogen, hydroxy, (1-6C)alkoxy, (3-7C)cycloalkyl-oxy and (3-7C)cycloalkyl-(1-6C)alkoxy (particularly hydrogen, hydroxy and (1-6C)alkoxy),

and wherein any CH₂ or CH₃ group within a R¹ substituent optionally bears on each said CH₂ or CH₃ group one or more halogeno or (1-6C)alkyl substituents, or a substituent selected from hydroxy, cyano, amino, carboxy, carbamoyl, sulfamoyl, oxo, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, N-(1-6C)alkylsulfamoyl and N,N-di-[(1-6C)alkyl]sulfamoyl, (1-6C)alkanesulfonylamino and N-(1-6C)alkyl-(1-6C)alkanesulfonylamino;

(b) R¹ is selected from hydrogen, hydroxy, (1-6C)alkoxy, (3-7C)cycloalkyl-oxy and (3-7C)cycloalkyl-(1-6C)alkoxy (particularly hydrogen, hydroxy and (1-6C)alkoxy),

and wherein any CH₂ or CH₃ group within a R¹ substituent optionally bears on each said CH₂ or CH₃ group one or more halogeno or (1-6C)alkyl substituents, or a substituent selected from hydroxy, cyano, amino, (1-6C)alkoxy, (1-6C)alkylamino and di-[(1-6C)alkyl]amino;

(c) R¹ is selected from hydrogen, hydroxy, (1-6C)alkoxy, (3-7C)cycloalkyl-oxy and (3-7C)cycloalkyl-(1-6C)alkoxy (particularly hydrogen, hydroxy and (1-6C)alkoxy),

and wherein any CH₂ or CH₃ group within a R¹ substituent optionally bears on each said CH₂ or CH₃ group one or more fluoro or chloro substituents, or a substituent selected from hydroxy, amino, (1-4C)alkoxy, (1-4C)alkylamino and di-[(1-4C)alkyl]amino;

(d) R¹ is selected from hydrogen, (1-6C)alkoxy, cyclopropyl-(1-4C)alkoxy, cyclobutyl-(1-4C)alkoxy, cyclopentyl-(1-4C)alkoxy and cyclohexyl-(1-6C)alkoxy,

and wherein any CH₂ or CH₃ group within a R¹ substituent optionally bears on each said CH₂ or CH₃ group one or more fluoro or chloro substituents, or a substituent selected from hydroxy, methoxy and ethoxy;

(e) R¹ is selected from hydrogen, hydroxyl and (1-6C)alkoxy,

and wherein any CH₂ or CH₃ group within a R¹ substituent optionally bears on each said CH₂ or CH₃ group one or more fluoro or chloro substituents, or a substituent selected from hydroxy, methoxy and ethoxy;

(f) R¹ is selected from hydrogen, (1-6C)alkoxy, cyclopropylmethoxy and 2-cyclopropylethoxy,

and wherein any CH₂ or CH₃ group within a R¹ substituent optionally bears on each said CH₂ or CH₃ group one or more fluoro or chloro substituents, or a substituent selected from hydroxy, methoxy and ethoxy;

(g) R¹ is selected from hydrogen, methoxy, ethoxy, propyloxy, isopropyloxy, cyclopropylmethoxy, 2-hydroxyethoxy, 2-fluoroethoxy, 2-methoxyethoxy, 2-ethoxyethoxy, 2,2-difluoroethoxy and 2,2,2-trifluoroethoxy;

(h) R¹ is selected from hydrogen and (1-3C)alkoxy;

(i) R¹ is hydrogen;

(j) R¹ is methoxy;

(k) Y is selected from hydrogen, halogeno, (1-4C)alkyl, (1-4C)alkoxy and (2-4C)alkynyl;

(l) Y is selected from halogeno, (1-4C)alkyl, (1-4C)alkoxy, (2-4C)alkenyl and (2-4C)alkynyl;

(m) Y is selected from halogeno, (1-4C)alkyl, (1-4C)alkoxy and (2-4C)alkynyl;

(n) Y is selected from hydrogen, halogeno, (1-4C)alkoxy and (2-4C)alkynyl;

(o) Y is selected from hydrogen, halogeno and (1-4C)alkoxy;

(p) Y is selected from hydrogen and halogeno;

(q) Y is halogeno;

(r) Y is selected from hydrogen, fluoro, chloro, methyl, methoxy and ethynyl;

(s) Y is selected from hydrogen, fluoro, chloro and methoxy;

(t) Y is selected from hydrogen, fluoro, chloro and methyl;

(u) Y is selected from hydrogen, fluoro, chloro and bromo;

(v) Y is selected from hydrogen, chloro and methoxy;

(w) Y is selected from hydrogen and chloro;

(x) Y is hydrogen;

(y) Y is chloro;

(z) Y is fluoro;

(aa) Y is methoxy;

(bb) Y is ethynyl;

(cc) Y is methyl;

(dd) a is 0, 1 or 2 and each R², which may be the same or different, is selected from halogeno;

(ee) a is 0 or 1 and R² is selected from fluoro and chloro;

(ff) a is 0;

(gg) a is 0 and Y is selected from hydrogen, fluoro, chloro, methyl, methoxy and ethynyl;

(hh) a is 0 and Y is halogeno, particularly chloro;

(ii) X² is selected from O, S and OC(R⁴)₂ wherein each R⁴ is, independently, hydrogen or (1-4C)alkyl;

(jj) X² is selected from O, S and OCH₂;

(kk) X² is O;

(ll) X² is S;

(mm) X² is OCH₂;

(nn) X² is OCH₂ and Y is halogeno, particularly chloro;

(oo) X² is OCH₂, X is chloro and a is 0;

(pp) X² is OCH₂ and Y is selected from hydrogen, halogeno, (1-4C)alkoxy and (2-4C)alkynyl;

(qq) X² is OCH₂ and Y is selected from hydrogen, chloro, methoxy and ethynyl;

(rr) X² is OCH₂, Y is selected from hydrogen, halogeno, (1-4C)alkoxy and (2-4C)alkynyl and a is 0;

(ss) X² is OCH₂, Y is selected from hydrogen, chloro, methoxy and ethynyl and a is 0;

(tt) X² is S and Y is selected from hydrogen and halogeno (particularly chloro or fluoro);

(uu) X² is S, Y is selected from hydrogen and halogeno (particularly chloro or fluoro) and a is 0;

(vv) X² is O and Y is (1-4C)alkyl (particularly (1-2C)alkyl, such as methyl);

(ww) X² is O and Y is (1-4C)alkyl (particularly (1-2C)alkyl, such as methyl) and a is 0;

(xx) Q² is selected from phenyl and a 5- or 6-membered monocyclic heteroaryl ring, which ring contains 1, 2 or 3 heteroatoms independently selected from oxygen, nitrogen and sulfur,

and wherein Q² optionally bears one or more substituents (for example 1, 2 or 3), which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C) alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, and a group of the formula:
—X⁴—R⁵

wherein X⁴ is a direct bond or is selected from O, CO and N(R⁶), wherein R⁶ is hydrogen or (1-6C)alkyl, and R⁵ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulfamoyl(1-6C)alkyl, N-(1-6C)alkylsulfamoyl(1-6C)alkyl, N,N di-(1-6C)alkylsulfamoyl(1-6C)alkyl, (2-6C)alkanoyl-(1-6C)alkyl, (2-6C)alkanoyloxy-(1-6C)alkyl or (1-6C)alkoxycarbonyl-(1-6C)alkyl,

and wherein any CH₂ or CH₃ group within Q² optionally bears on each said CH₂ or CH₃ one or more (for example 1, 2, or 3) halogeno or (1-6C)alkyl substituents or a substituent selected from hydroxy, cyano, amino, (1-4C)alkoxy, (1-4C)alkylamino and di-[(1-4C)alkylamino];

(yy) Q² is selected from phenyl and a 5- or 6-membered monocyclic heteroaryl ring, which ring contains 1 nitrogen heteroatom and optionally 1 or 2 (particularly 1) additional heteroatom independently selected from oxygen, nitrogen and sulfur,

and wherein Q² optionally bears one or more substituents (for example 1, 2 or 3), which may be the same or different, as hereinbefore defined in (xx);

(zz) Q² is phenyl,

and wherein Q² optionally bears one or more substituents (for example 1, 2 or 3), which may be the same or different, as hereinbefore defined in (xx);

(aaa) Q² is a 5- or 6-membered monocyclic heteroaryl ring, which ring contains 1 nitrogen heteroatom and optionally 1 additional heteroatom selected from oxygen, nitrogen and sulfur,

and wherein Q² optionally bears one or more substituents (for example 1, 2 or 3), which may be the same or different, as hereinbefore defined in (xx);

(bbb) Q² is a 5- or 6-membered monocyclic heteroaryl ring, which ring contains 1 nitrogen heteroatom and optionally 1 additional nitrogen heteroatom,

and wherein Q² optionally bears one or more substituents (for example 1, 2 or 3), which may be the same or different, as hereinbefore defined in (xx);

(ccc) Q² is selected from phenyl, pyridyl, pyrazinyl, 1,3-thiazolyl, 1H-imidazolyl, 1H-pyrazolyl, 1,3-oxazolyl and isoxazolyl,

and wherein Q² optionally bears one or more substituents (for example 1, 2 or 3), which may be the same or different, as hereinbefore defined in (xx);

(ddd) Q² is selected from phenyl, pyridyl, pyrazinyl, 1,3-thiazolyl and 1H-imidazolyl,

and wherein Q² optionally bears one or more substituents (for example 1, 2 or 3), which may be the same or different, as hereinbefore defined in (xx);

(eee) Q² is selected from pyridyl, pyrazinyl, 1,3-thiazolyl and isoxazolyl,

and wherein Q² optionally bears one or more substituents (for example 1, 2 or 3), which may be the same or different, as hereinbefore defined in (xx);

(fff) Q² is selected from phenyl, pyridyl and pyrazinyl,

and wherein Q² optionally bears one or more substituents (for example 1, 2 or 3), which may be the same or different, as hereinbefore defined in (xx);

(ggg) Q² is selected from phenyl, 2-, 3- or 4-pyridyl, 2-pyrazinyl, 1H-imidazol-2-yl, 1,3-thiazol-2-yl, 1,3-thiazol-4-yl, 1,3-thiazol-5-yl, 3-isoxazolyl, 4-isoxazolyl and 5-isoxazolyl,

and wherein Q² optionally bears one or more substituents (for example 1, 2 or 3), which may be the same or different, as hereinbefore defined in (xx);

(hhh) Q² is selected from phenyl, 2-pyridyl, 3-pyridyl, 2-pyrazinyl, 1H-imidazol-2-yl and 1,3-thiazol-2-yl,

and wherein Q² optionally bears one or more substituents (for example 1, 2 or 3), which may be the same or different, as hereinbefore defined in (xx);

(iii) Q² is selected from 2-, 3- or 4-pyridyl, 2-pyrazinyl, 1,3-thiazol-2-yl, 1,3-thiazol-4-yl, 1,3-thiazol-5-yl, 3-isoxazolyl, 4-isoxazolyl and 5-isoxazolyl,

and wherein Q² optionally bears one or more substituents (for example 1, 2 or 3), which may be the same or different, as hereinbefore defined in (xx);

(jjj) Q² is selected from 2-pyridyl, 2-pyrazinyl, 1,3-thiazol-4-yl, 1,3-thiazol-5-yl and 3-isoxazolyl,

and wherein Q² optionally bears one or more substituents (for example 1, 2 or 3), which may be the same or different, as hereinbefore defined in (xx);

(kkk) Q² is selected from phenyl, 2-pyridyl and 2-pyrazinyl,

and wherein Q² optionally bears 1 or 2 substituents selected from halogeno (particularly fluoro);

(lll) Q² is pyrazinyl particularly 2-pyrazinyl), which optionally bears one or more substituents (for example 1, 2 or 3), which may be the same or different, as defined above in (xx);

(mmm) Q² is isoxazolyl particularly isoxazol-3-yl), which optionally bears one or more substituents (for example 1, 2 or 3), which may be the same or different, as defined above in (xx);

(nnn) Q² is pyridyl (particularly 2-pyridyl or 3-pyridyl, more particularly 2-pyridyl), which optionally bears one or more substituents (for example 1, 2 or 3), which may be the same or different, as defined above in (xx);

(ooo) Q² is 1,3-thiazolyl (particularly 1,3-thiazol-2-yl, 1,3-thiazol-4-yl or 1,3-thiazolyl-5-yl, more particularly 1,3-thiazol-2-yl), which optionally bears one or more substituents (for example 1, 2 or 3), which may be the same or different, as defined above in (xx);

(ppp) Q² is phenyl, which optionally bears one or more substituents (for example 1, 2 or 3), which may be the same or different, as defined above in (xx);

(qqq) Q² is 1H-imidazolyl (particularly 1H-imidazol-2-yl), which optionally bears one or more substituents (for example 1, 2 or 3), which may be the same or different, as defined above in (xx);

(rrr) Q² is selected from phenyl and a 5- or 6-membered monocyclic heteroaryl ring, which ring contains 1, 2 or 3 heteroatoms independently selected from oxygen, nitrogen and sulfur,

and wherein Q² optionally bears one or more substituents (for example 1, 2 or 3), which may be the same or different, selected from halogeno, hydroxy, amino, carbamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl and (2-6C)alkanoyloxy;

(sss) Q² is selected from phenyl and a 5- or 6-membered monocyclic heteroaryl ring, which ring contains 1 nitrogen heteroatom and optionally 1 or 2 particularly 1) additional heteroatom independently selected from oxygen, nitrogen and sulfur,

and wherein Q² optionally bears one or more substituents (for example 1, 2 or 3), which may be the same or different, as hereinbefore defined in (rrr);

(ttt) Q² is phenyl,

and wherein Q² optionally bears one or more substituents (for example 1, 2 or 3), which may be the same or different, as hereinbefore defined in (rrr);

(uuu) Q² is a 5- or 6-membered monocyclic heteroaryl ring, which ring contains 1 nitrogen heteroatom and optionally 1 additional heteroatom selected from oxygen, nitrogen and sulfur,

and wherein Q² optionally bears one or more substituents (for example 1, 2 or 3), which may be the same or different, as hereinbefore defined in (rrr);

(vvv) Q² is a 5- or 6-membered monocyclic heteroaryl ring, which ring contains 1 nitrogen heteroatom and optionally 1 additional nitrogen heteroatom,

and wherein Q² optionally bears one or more substituents (for example 1, 2 or 3), which may be the same or different, as hereinbefore defined in (rrr);

(www) Q² is selected from phenyl, pyridyl, pyrazinyl, 1,3-thiazolyl, 1H-imidazolyl, 1H-pyrazolyl, 1,3-oxazolyl and isoxazolyl,

and wherein Q² optionally bears one or more substituents (for example 1, 2 or 3), which may be the same or different, as hereinbefore defined in (rrr);

(xxx) Q² is selected from phenyl, pyridyl, pyrazinyl, 1,3-thiazolyl and 1H-imidazolyl,

and wherein Q² optionally bears one or more substituents (for example 1, 2 or 3), which may be the same or different, as hereinbefore defined in (rrr);

(yyy) Q² is selected from phenyl, pyridyl and pyrazinyl,

and wherein Q² optionally bears one or more substituents (for example 1, 2 or 3), which may be the same or different, as hereinbefore defined in (rrr);

(zzz) Q² is selected from phenyl, 2-, 3- or 4-pyridyl, 2-pyrazinyl, 1H-imidazol-2-yl, 1,3-thiazol-2-yl, 1,3-thiazol-4-yl, 1,3-thiazol-5-yl, 3-isoxazolyl, 4-isoxazolyl and 5-isoxazolyl,

and wherein Q² optionally bears one or more substituents (for example 1, 2 or 3), which may be the same or different, as hereinbefore defined in (rrr);

(aaaa) Q² is selected from phenyl, 2-pyridyl, 3-pyridyl, 2-pyrazinyl, 1H-imidazol-2-yl and 1,3-thiazol-2-yl,

and wherein Q² optionally bears one or more substituents (for example 1, 2 or 3), which may be the same or different, as hereinbefore defined in (rrr);

for example 1, 2 or 3), which may be the same or different, as hereinbefore defined in (ii);

(bbbb) Q² is selected from phenyl, 2-pyridyl and 2-pyrazinyl,

and wherein Q² optionally bears 1 or 2 substituents selected from halogeno (particularly fluoro);

(cccc) Q² is pyrazinyl particularly 2-pyrazinyl), which optionally bears one or more substituents (for example 1, 2 or 3), which may be the same or different, as defined above in (rrr);

(dddd) Q² is isoxazolyl (particularly isoxazol-3-yl), which optionally bears one or more substituents (for example 1, 2 or 3), which may be the same or different, as defined above in (rrr);

(eeee) Q² is pyridyl (particularly 2-pyridyl or 3-pyridyl, more particularly 2-pyridyl), which optionally bears one or more substituents (for example 1, 2 or 3), which may be the same or different, as defined above in (rrr);

(ffff) Q² is 1,3-thiazolyl (particularly 1,3-thiazol-2-yl, 1,3-thiazol-4-yl or 1,3-thiazolyl-5-yl, more particularly 1,3-thiazol-2-yl), which optionally bears one or more substituents (for example 1, 2 or 3), which may be the same or different, as defined above in (rrr);

(gggg) Q² is phenyl, which optionally bears one or more substituents (for example 1, 2 or 3), which may be the same or different, as defined above in (rrr);

(hhhh) Q² is 1H-imidazolyl (particularly 1H-imidazol-2-yl), which optionally bears one or more substituents (for example 1, 2 or 3), which may be the same or different, as defined above in (rrrr);

(iiii) Q² is selected from phenyl and a 5- or 6-membered monocyclic heteroaryl ring, which ring contains 1, 2 or 3 heteroatoms independently selected from oxygen, nitrogen and sulfur,

and wherein. Q² optionally bears one or more substituents. (for example 1, 2 or 3), which may be the same or different, selected from halogeno, hydroxy, (1-6C)alkyl, (2-8C)alkenyl, (2-8C)alkynyl and (1-6C)alkoxy;

(jjjj) Q² is selected from phenyl and a 5- or 6-membered monocyclic heteroaryl ring, which ring contains 1 nitrogen heteroatom and optionally 1 or 2 (particularly 1) additional heteroatom independently selected from oxygen, nitrogen and sulfur,

and wherein Q² optionally bears one or more substituents (for example 1, 2 or 3), which may be the same or different, as hereinbefore defined in (iiii);

(kkkk) Q¹ is phenyl,

and wherein Q² optionally bears one or more substituents (for example 1, 2 or 3), which may be the same or different, as hereinbefore defined in (iiii);

(llll) Q² is a 5- or 6-membered monocyclic heteroaryl ring, which ring contains 1 nitrogen heteroatom and optionally 1 additional heteroatom selected from oxygen, nitrogen and sulfur,

and wherein Q² optionally bears one or more substituents (for example 1, 2 or 3), which may be the same or different, as hereinbefore defined in (iiii);

(mmmm) Q² is selected from phenyl, pyridyl, pyrazinyl, 1,3-thiazolyl and 1H-imidazolyl,

and wherein Q² optionally bears one or more substituents (for example 1, 2 or 3), which may be the same or different, as hereinbefore defined in (iiii);

(nnnn) Q² is selected from phenyl, 2-pyridyl, 3-pyridyl, 2-pyrazinyl, 1H-imidazol-2-yl and 1,3-thiazol-2-yl,

and wherein Q² optionally bears one or more substituents (for example 1, 2 or 3), which may be the same or different, as hereinbefore defined in (iiii);

(oooo) Q² is selected from phenyl, 2-pyridyl, 2-pyrazinyl, 1,3-thiazol-4-yl, 1,3-thiazol-5-yl, and isoxazol-3-yl,

and wherein Q² optionally bears one or more substituents (for example 1, 2 or 3), which may be the same or different selected from halogeno, hydroxy, cyano, carboxy, nitro, amino, (1-4C)alkyl, (1-4C)alkoxy, (2-4C)alkenyl, (2-4C)alkynyl, (1-4C)alkylthio, (1-4C)alkylsulfinyl, (1-4C)alkylsulfonyl, (2-4C)alkanoyl, N-(1-4C)alkylamino, N,N-di-[(1-4C)alkyl]amino, (1-4C)alkoxycarbonyl, carbamoyl, N-(1-4C)alkylcarbamoyl, N. N-di-[(1-4C)alkyl]carbamoyl, (2-4C)alkanoyloxy, (2-4C)alkanoylamino, N-(1-4C)alkyl-(2-4C)alkanoylamino, halogeno-(1-4C)alkyl, hydroxy-(1-4C)alkyl, (1-4C)alkoxy-(1-4C)alkyl, cyano-(1-4C)alkyl, carboxy-(1-4C)alkyl, amino-(1-4C)alkyl, N-(1-4C)alkylamino-(1-4C)alkyl and N,N-di-[(1-4C)alkyl]amino-(1-4C)alkyl;

(pppp) Q² is selected from phenyl, 2-pyridyl, 3-pyridyl, 2-pyrazinyl, 1,3-thiazol-2-yl and 1H-imidazol-2-yl,

and wherein Q² optionally bears one or more substituents (for example 1, 2 or 3), which may be the same or different, as hereinbefore defined in (oooo);

(qqqq) Q² is selected from phenyl, 2-pyridyl, 3-pyridyl and 2-pyrazinyl,

and wherein Q² optionally bears one or more substituents (for example 1, 2 or 3), which may be the same or different, as hereinbefore defined in (oooo);

(rrrr) Q² is selected from phenyl, 2-pyridyl, 2-pyrazinyl, 1,3-thiazol-4-yl, 1,3-thiazol-5-yl and isoxazol-3-yl,

and wherein Q² optionally bears one or more substituents (for example 1, 2 or 3), which may be the same or different, selected from fluoro, chloro, bromo, hydroxy, carboxy, cyano, nitro, amino, methyl, ethyl, isopropyl, methoxy, ethoxy, vinyl, allyl, ethynyl, 2-propynyl, methylthio, methylsulfinyl, methylsulfonyl, acetyl, propionyl, methylamino, ethylamino, N,N-dimethylamino, N,N-diethylamino, N-methyl-N-ethylamino methoxycarbonyl, ethoxycarbonyl, carbamoyl, N-methylcarbamoyl, N,N-dimethylcarbamoyl, acetoxy, acetamido, fluoromethyl, 2-fluoroethyl, chloromethyl, 2-chloroethyl, hydroxymethyl, 2-hydroxyethyl, methoxymethyl, 2-methoxyethyl, cyanomethyl, 2-cyanoethyl, carboxymethyl, 2-carboxymethyl, aminomethyl, methylaminomethyl, ethylaminomethyl, N,N-dimethylaminomethyl, N,N-diethylaminomethyl, N-methyl-N-ethylaminomethyl, 2-aminoethyl, 2-(methylamino)ethyl, 2-(ethylamino)ethyl, 2-(N,N-dimethylamino)ethyl, 2-(N,N-diethylamino)ethyl, 2-(N-methyl-N-ethylamino)ethyl, carbamoylmethyl, N-methylcarbamoylmethyl and N,N-dimethylcarbamoylmethyl;

(ssss) Q² is selected from phenyl, 2-pyridyl, 3-pyridyl, 2-pyrazinyl, 1,3-thiazol-2-yl and 1H-imidazol-2-yl,

and wherein Q² optionally bears one or more substituents (for example 1, 2 or 3), which may be the same or different, as hereinbefore defined in (rrrr);

(tttt) Q² is selected from phenyl, 2-pyridyl, 3-pyridyl and 2-pyrazinyl,

and wherein Q² optionally bears one or more substituents (for example 1, 2 or 3), which may be the same or different, as hereinbefore defined in (rrrr);

(uuuu) Q² is selected from 2-pyridyl, 2-pyrazinyl, 1,3-thiazol-4-yl, 1,3-thiazol-5-yl and isoxazol-3-yl,

and wherein Q² optionally bears 1, 2, or 3 substituents, which may be the same or different, as defined above in (rrrr);

(vvvv) Q² is selected from phenyl, 2-pyridyl and 2-pyrazinyl,

and wherein Q² optionally bears 1, 2, or 3 substituents, which may be the same or different, selected from fluoro, chloro, hydroxy, cyano, nitro, (1-4C)alkyl and (1-4C)alkoxy;

(wwww) Q² is phenyl which optionally bears one or more substituents (for example 1, 2, or

3), which may be the same or different, selected from fluoro, chloro, bromo, cyano, methyl and methoxy;

(xxxx) Q² is phenyl which bears 1 or 2 substituents, which may be the same or different, selected from halogeno (particularly fluoro and chloro, more particularly fluoro);

(yyyy) Q² is 3-fluorophenyl;

(zzzz) Q² is pyridyl which optionally bears 1 or 2 substituents selected from fluoro, chloro, hydroxy, (1-4C)alkyl and (1-4C)alkoxy;

(aaaaa) Q² is 2-pyridyl which optionally bears 1 or 2 substituents selected from fluoro, chloro, hydroxy, (1-4C)alkyl and (1-4C)alkoxy;

(bbbbb) Q² is 3-pyridyl which optionally bears 1 or 2 substituents selected from fluoro, chloro, hydroxy, (1-4C)alkyl and (1-4C)alkoxy;

(ccccc) Q² is selected from 2-pyridyl and 6-methylpyrid-3-yl;

(ddddd) Q² is 2-pyridyl;

(eeeee) Q² is 6-methylpyrid-3-yl;

(fffff) Q² is 2-pyrazinyl which optionally bears 1 or 2 substituents, which may be the same or different, selected from fluoro, chloro, hydroxy, (1-4C)alkyl and (1-4C)alkoxy;

(ggggg) Q² is 2-pyrazinyl;

(hhhhh) Q² is 1,3-thiazol-2-yl which optionally bears 1 or 2 substituents, which may be the same or different, selected from fluoro, chloro, hydroxy, (1-4C)alkyl and (1-4C)alkoxy;

(iiiii) Q² is 1,3-thiazol-2-yl;

(jjjjj) Q² is 1-methyl-1H-imidazol-2-yl which optionally bears 1 or 2 substituents, which may be the same or different, selected from fluoro, chloro, hydroxy, (1-4C)alkyl and (1-4C)alkoxy;

(kkkkk) Q² is 1H-imidazol-2-yl;

(lllll) Q² is selected from 2-pyridyl, 6-methyl-pyrid-3yl, 3-fluorophenyl, 2-pyrazinyl, 1,3-thiazol-2-yl and 1-methyl-1H-imidazol-2-yl;

(mmmmm) Q² is selected from 3-fluorophenyl, 2-pyridyl and 2-pyrazinyl,

(nnnnn) Q² is selected from phenyl, 2-pyridyl, 3-pyridyl, 2-pyrazinyl, 1,3-thiazol-2-yl, and 1H-imidazol-2-yl,

and wherein Q² optionally bears 1, 2, or 3 substituents, which may be the same or different, selected from fluoro, chloro, hydroxy, cyano, nitro, amino, (1-4C)alkyl, (1-4C)alkoxy, N-(1-4C)alkylamino and N,N-di-[(1-4C)alkyl]amino,

and X² is selected from OCH₂, O and S;

(ooooo) Q² is selected from phenyl, 2-pyridyl, 2-pyrazinyl, 1,3-thiazolyl, 1,3-thiazol-5-yl and isoxazol-3-yl,

and wherein Q² optionally bears 1, 2, or 3 substituents, which may be the same or different, selected from fluoro, chloro, hydroxy, cyano, nitro, amino, (1-4C)alkyl, (1-4C)alkoxy, N-(1-4C)alkylamino and N,N-di-[(1-4C)alkyl]amino,

and X² is OCH₂;

(ppppp) Q² is selected from phenyl, 2-pyridyl and 2-pyrazinyl,

and wherein Q² optionally bears 1, 2, or 3 substituents, which may be the same or different, selected from fluoro, chloro, hydroxy, cyano, nitro, amino, (1-4C)alkyl, (1-4C)alkoxy, N-(1-4C)alkylamino and N,N-di-[(1-4C)alkyl]amino,

X² is OCH₂, and

a is 0;

(qqqqq) Q² is selected from 1,3-thiazol-2-yl and 1H-imidazol-2-yl,

and wherein Q² optionally bears 1, 2, or 3 substituents, which may be the same or different, selected from fluoro, chloro, hydroxy, cyano, nitro, amino, (1-4C)alkyl, (1-4C)alkoxy, N-(1-4C)alkylamino and N,N-di-[(1-4C)alkyl]amino,

and X² is S;

(rrrrr) Q² is 3-pyridyl,

and wherein Q² optionally bears 1, 2, or 3 substituents, which may be the same or different, selected from fluoro, chloro, hydroxy, cyano, nitro, amino, (1-4C)alkyl, (1-4C)alkoxy, N-(1-4C)alkylamino and N,N-di-[(1-4C)alkyl]amino,

and X² is O;

(sssss) Q² is selected from 2-pyridyl and 3-pyridyl particularly 2-pyridyl),

and wherein Q² optionally bears a (1-4C)alkyl substituent (for example methyl),

X² is O,

a is 0, and

Y is (1-4C)alkyl (for example methyl);

(ttttt) Q² is selected from phenyl, 2-pyridyl, 3-pyridyl, 2-pyrazinyl, 1,3-thiazol-2-yl and 1H-imidazol-2-yl,

and wherein Q² optionally bears 1, 2, or 3 substituents, which may be the same or different, selected from fluoro, chloro, hydroxy, cyano, nitro, amino, (1-4C)alkyl, (1-4C)alkoxy, N-(1-4C)alkylamino and N,N-di-[(1-4C)alkyl]amino,

X² is selected from OCH₂, O and S,

a is 0; and

Y is selected from hydrogen, fluoro, chloro, methyl, methoxy and ethynyl;

(uuuu) Q² is selected from phenyl, 2-pyridyl, 3-pyridyl, 2-pyrazinyl, 1,3-thiazol-2-yl and 1H-imidazol-2-yl,

and wherein Q² optionally bears 1, 2, or 3 substituents, which may be the same or different, selected from fluoro, chloro, hydroxy, cyano, nitro, amino, (1-4C)alkyl, (1-4C)alkoxy, N-(1-4C)alkylamino and N,N-di-[(1-4C)alkyl]amino,

X² is OCH₂,

a is 0; and

Y is selected from hydrogen, chloro, methoxy and ethynyl;

(vvvvv) Q² is selected from phenyl, 2-pyridyl, 3-pyridyl, 2-pyrazinyl, 1,3-thiazol-2-yl and 1H-imidazol-2-yl,

and wherein Q² optionally bears 1, 2, or 3 substituents, which may be the same or different, selected from fluoro, chloro, hydroxy, cyano, nitro, amino, (1-4C)alkyl, (1-4C)alkoxy, N-(1-4C)alkylamino and N,N-di-[(1-4C)alkyl]amino,

X² is O,

a is 0; and

Y is methyl;

(wwwww) Q² is selected from phenyl, 2-pyridyl, 3-pyridyl, 2-pyrazinyl, 1,3-thiazol-2-yl and 1H-imidazol-2-yl,

and wherein Q² optionally bears 1, 2, or 3 substituents, which may be the same or different, selected from fluoro, chloro, hydroxy, cyano, nitro, amino, (1-4C)alkyl, (1-4C)alkoxy, N-(1-4C)alkylamino and N. N-di-[(1-4C)alkyl]amino,

X² is s,

a is 0; and

Y is selected from hydrogen, fluoro and chloro;

(xxxxx) X¹ is selected from a direct bond and CH₂;

(yyyyy) a X¹ is C(R⁷)₂, wherein each R⁷, which may be the same or different, is selected from hydrogen and (1-4C)alkyl;

(zzzzz) X¹ is CH₂;

(aaaaaa) X¹ is a direct bond;

(bbbbbb) Q¹ is selected from azetidinyl, pyrrolidinyl, piperidinyl, homopiperidinyl, piperazinyl, morpholinyl and thiomorpholinyl,

and wherein Q¹ is linked to the group X¹—O by a ring carbon atom,

and wherein Q¹ optionally bears one or more (for example 1, 2 or 3) substituents, which may be the same or different, selected from halogeno, trifluoromethyl, hydroxy, carbamoyl, (1-4C)alkyl, (1-4C)alkoxy, N-(1-4C)alkylcarbamoyl and N,N-di-[(1-4C)alkyl]carbamoyl,

and wherein any heterocyclyl group within Q¹ optionally bears an oxo substituent;

(cccccc) Q¹ is selected from azetidinyl, pyrrolidinyl and piperidinyl,

and wherein Q¹ is linked to the group X¹—O by a ring carbon atom,

and wherein Q¹ optionally bears one or more (for example 1, 2 or 3) substituents, which may be the same or different, selected from halogeno, trifluoromethyl, hydroxy, carbamoyl, (1-4C)alkyl, (1-4C)alkoxy, N-(1-4C)alkylcarbamoyl and N,N-di-[(1-4C)alkyl]carbamoyl,

and wherein any heterocyclyl group within Q¹ optionally bears an oxo substituent;

(dddddd) Q¹ is selected from azetidin-2-yl, azetidin-3-yl, pyrrolidin-2-yl, pyrrolidin-3-yl, morpholin-2-yl, morpholin-3-yl, thiomorpholin-2-yl, thiomorpholin-3-yl, piperidin-2-yl, piperidin-3-yl, piperidin-4-yl, piperazin-2-yl, 2-, 3- or 4-homopiperidinyl, 2, 3, 5, 6 or 7-homopiperazinyl,

and wherein Q¹ optionally bears one or more (for example 1, 2 or 3) substituents, which may be the same or different, selected from fluoro, chloro, hydroxy, carbamoyl, (1-4C)alkyl, (1-4C)alkoxy, N-(1-4C)alkylcarbamoyl and N,N-di-[(1-4C)alkyl]carbamoyl,

and wherein any heterocyclyl group within Q¹ optionally bears an oxo substituent;

(eeeeee) Q¹ is selected from azetidin-3-yl, pyrrolidin-2-yl, pyrrolidin-3-yl, piperidin-3-yl or piperidin-4-yl,

and wherein Q¹ optionally bears one or more (for example 1, 2 or 3) substituents, which may be the same or different, selected from fluoro, chloro, hydroxy, carbamoyl, (1-4C)alkyl, (1-4C)alkoxy, N-(1-4C)alkylcarbamoyl and N,N-di-[(1-4C)alkyl]carbamoyl,

and wherein any heterocyclyl group within Q¹ optionally bears an oxo substituent;

(ffffff) Q¹ is piperidinyl,

and wherein Q¹ optionally bears one or more (for example 1, 2 or 3) substituents, which may be the same or different, selected from fluoro, chloro, hydroxy, carbamoyl, (1-4C)alkyl, (1-4C)alkoxy, N-(1-4C)alkylcarbamoyl and N,N-di-[(1-4C)alkyl]carbamoyl,

and wherein the piperidinyl group within Q¹ optionally bears an oxo substituent;

(gggggg) Q¹ is selected from pyrrolidin-2-yl, pyrrolidin-3-yl, morpholin-2-yl, morpholin-3-yl, piperidin-2-yl, piperidin-3-yl and piperazin-2-yl,

and wherein Q¹ optionally bears one or more (for example 1, 2 or 3) substituents, which may be the same or different, selected from fluoro, chloro, hydroxy, carbamoyl, (1-4C)alkyl, (1-4C)alkoxy, N-(1-4C)alkylcarbamoyl and N,N-di-[(1-4C)alkyl]carbamoyl,

and wherein any heterocyclyl group within Q¹ optionally bears an oxo substituent;

(hhhhhh) Q¹ is selected from pyrrolidin-2-yl, pyrrolidin-3-yl, piperidin-2-yl, piperidin-3-yl and piperidin-4-yl

and wherein Q¹ optionally bears one or more (for example 1, 2 or 3) substituents, which may be the same or different, selected from fluoro, chloro, hydroxy, carbamoyl, (1-4C)alkyl, (1-4C)alkoxy, N-(1-4C)alkylcarbamoyl and N,N-di-[(1-4C)alkyl]carbamoyl,

and wherein any heterocyclyl group within Q¹ optionally bears an oxo substituent;

(iiiiii) Q¹ is selected from pyrrolidin-2-yl and piperidin-2-yl,

and wherein Q¹ optionally bears one or more (for example 1, 2 or 3) substituents, which may be the same or different, selected from fluoro, chloro, hydroxy, carbamoyl, (1-4C)alkyl, (1-4C)alkoxy, N-(1-4C)alkylcarbamoyl and N,N-di-[(1-4C)alkyl]carbamoyl,

and wherein any heterocyclyl group within Q¹ optionally bears an oxo substituent;

(jjjjjj) Q¹ is pyrrolidin-2-yl,

and wherein Q¹ optionally bears 1 or 2 substituents, which may be the same or different, selected from fluoro, hydroxy, oxo, (1-4C)alkyl and (1-4C)alkoxy;

(kkkkkk) Q¹ is selected from piperidin-2-yl, piperidin-3-yl and piperidin-4-yl,

and wherein Q¹ optionally bears 1 or 2 substituents, which may be the same or different, selected from fluoro, hydroxy, oxo, (1-4C)alkyl and (1-4C)alkoxy;

(llllll) Q¹ is selected from piperidin-4-yl,

and wherein Q¹ optionally bears 1 or 2 substituents, which may be the same or different, selected from fluoro, hydroxy, oxo, (1-4C)alkyl and (1-4C)alkoxy;

(mmmmmm) Q¹ is pyrrolidin-2-yl;

(nnnnnn) Q¹ is pyrrolidin-3-yl;

(oooooo) Q¹ is piperidin-2-yl;

(pppppp) Q¹ is piperidin-3-yl;

(qqqqqq) Q¹ is piperidin-4-yl;

(rrrrrr) Q¹ is azetidin-3-yl

(ssssss) Q¹ is selected from azetidin-3-yl, pyrrolidin-2-yl, pyrrolidin-3-yl, piperidin-3-yl or piperidin-4-yl,

and wherein Q¹ optionally bears one or more (for example 1, 2 or 3) substituents, which may be the same or different, selected from fluoro, chloro, hydroxy, carbamoyl, (1-4C)alkyl, (1-4C)alkoxy, N-(1-4C)alkylcarbamoyl and N,N-di-[(1-4C)alkyl]carbamoyl,

and wherein any heterocyclyl group within Q¹ optionally bears an oxo substituent,

and X¹ is selected from a direct bond and CH₂;

(tttttt) Q¹-X¹ is selected from piperidin-4-yl, piperidin-3-yl, azetidin-3-yl, pyrrolidin-2-ylmethyl and pyrrolidin-3-ylmethyl,

and wherein Q¹ optionally bears 1 or 2 substituents, which may be the same or different, selected from fluoro, hydroxy, oxo, (1-4C)alkyl and (1-4C)alkoxy;

(uuuuuu) Q¹-X¹ is selected from pyrrolidin-2-ylmethyl, pyrrolidin-3-ylmethyl, morpholin-2-ylmethyl, morpholin-3-ylmethyl, piperidin-2-ylmethyl, piperidin-3-ylmethyl, piperidin-4-ylmethyl and piperazin-2-ylmethyl,

and wherein Q¹ optionally bears 1 or 2 substituents, which may be the same or different, selected from fluoro, hydroxy, oxo, (1-4C)alkyl and (1-4C)alkoxy;

(vvvvvv) Q¹-X¹ is selected from pyrrolidin-2-ylmethyl and pyrrolidin-3-ylmethyl,

and wherein Q¹ optionally bears 1 or 2 substituents, which may be the same or different, selected from hydroxy, oxo, (1-4C)alkyl and (1-4C)alkoxy;

(wwwwww) Q¹-X¹ is selected from piperidin-4-yl and piperidin-3-yl,

and wherein Q¹ optionally bears 1 or 2 substituents, which may be the same or different, selected from hydroxy, oxo, (1-4C)alkyl and (1-4C)alkoxy;

(xxxxxx) Q¹-X¹ is azetidin-3-yl,

and wherein Q¹ optionally bears 1 or 2 substituents, which may be the same or different, selected from hydroxy, oxo, (1-4C)alkyl and (1-4C)alkoxy;

(yyyyyy) The group Q¹-X¹—O— is selected from pyrrolidin-3-yloxy, piperidin-3-yloxy and piperidinyloxy,

and wherein the piperidinyl group optionally bears 1 or 2 substituents, which may be the same or different, selected from fluoro, chloro, hydroxy, oxo, (1-3C)alkyl and (1-3C)alkoxy;

(zzzzzz) Q¹-X¹ is piperidin-4-ylmethyl, and wherein the piperidinyl group optionally bears 1 or 2 substituents, which may be the same or different, selected from fluoro, chloro, hydroxy, oxo, (1-3C)alkyl and (1-3C)alkoxy;

(aaaaaaa) Q¹-X¹ is piperidin-4-yl;

(bbbbbbb) Q¹-X¹ is piperidin-3-yl;

(ccccccc) Q¹-X¹ is azetidin-3-yl;

(ddddddd) Q¹-X¹ is pyrrolidin-2-ylmethyl;

(eeeeeee) Q¹-X¹ is pyrrolidin-3-ylmethyl;

For the avoidance of any doubt, the rings represented by Q¹ described in (bbbbbb) to (eeeeeee) above are all substituted on the ring nitrogen by the group Z-X³-M- in accordance with Formula I;

(ffffff) M is CO;

(ggggggg) M is SO₂;

(hhhhhhh) X³ is selected from a group of the formula -(Q³)ₘ-(CR¹⁰R¹¹)_{q}— and a group of the formula —(CR⁸R⁹)_{q}-(Q³)ₘ-, wherein m is 0 or 1, q is 1, 2, 3 or 4, and Q³, R⁸, R⁹, R¹⁰ and R¹¹ are as hereinbefore defined;

(iiiiiii) X³ is a group of the formula -Q³-, for example (3-7C)cycloalkylene such as cyclopropylidene;

(jjjjjjj) X³ is selected from cyclopropylene, cyclobutylene, cyclopentylene, cyclohexylene, methylene-(3-6C)cycloalkylene, (3-6C)cycloalkylene-methylene-, ethylene-(3-6C)cycloalkylene and (3-6C)cycloalkylene-ethylene-,

and wherein any CH₂ or CH₃ group within an X³ group, optionally bears on each said CH₂ or CH₃ group one or more halogeno substituents,

and wherein any CH₂ group which is attached to 2 carbon atoms or any CH₃ group which is attached to a carbon atom within a X³ substituent optionally bears on each said CH₂ or CH₃ group a substituent selected from hydroxy, and (1-6C)alkoxy;

(kkkkkkk) X³ is a group of the formula —(CR⁸R⁹)_{q}—,

q is 1, 2, 3 or 4,

each of R⁸ and R⁹, which may be the same or different, is selected from hydrogen and (1-6C)alkyl,

and wherein any CH₂ or CH₃ group within an X³ group, optionally bears on each said CH₂ or CH₃ group one or more halogeno substituents,

and wherein any CH₂ group which is attached to 2 carbon atoms or any CH₃ group which is attached to a carbon atom within a X³ substituent optionally bears on each said CH₂ or CH₃ group a substituent selected from hydroxy, and (1-6C)alkoxy;

(lllllll) X³ is a group of the formula —(CR⁸R⁹)_{q}—,

q is 1, 2, 3 or 4,

each of R⁸ and R⁹, which may be the same or different, is selected from hydrogen and (1-6C)alkyl, provided that at least one of R⁸ or R⁹ group in X³ is (1-6C)alkyl,

and wherein any CH₂ or CH₃ group within an X³ group, optionally bears on each said CH₂ or CH₃ group one or more halogeno substituents,

and wherein any CH₂ group which is attached to 2 carbon atoms or any CH₃ group which is attached to a carbon atom within a X³ substituent optionally bears on each said CH₂ or CH₃ group a substituent selected from hydroxy, and (1-6C)alkoxy;

(mmmmmmm) X³ is selected from a group of the formula —(CR⁸R⁹)—, —(CR⁸R⁹CH₂)—, —(CR⁸R⁹CH₂CH₂)—, —(CH₂CR⁸R⁹)— and —(CH₂CH₂CR⁸R⁹)—,

each of R⁸ and R⁹, which may be the same or different, is selected from hydrogen and (1-6C)alkyl, provided that at least one of R⁸ or R⁹ group in X³ is (1-6C)alkyl,

and wherein any CH₂ or CH₃ group within an X³ group, optionally bears on each said CH₂ or CH₃ group one or more halogeno substituents,

and wherein any CH₂ group which is attached to 2 carbon atoms or any CH₃ group which is attached to a carbon atom within a X³ substituent optionally bears on each said CH₂ or CH₃ group a substituent selected from hydroxy and (1-6C)alkoxy;

(nnnnnnn) X³ is selected from a group of the formula —(CR⁸R⁹)—, —(CR⁸R⁹CH₂)—, —(CR⁸R⁹CH₂CH₂)—, —(CH₂CR⁸R⁹)— and —(CH₂CH₂CR⁸R⁹)—,

each of R⁸ and R⁹, which may be the same or different, is selected from hydrogen and (1-6C)alkyl, provided that at least one of R⁸ or R⁹ group in X³ is a branched (1-6C)alkyl group,

and wherein any CH₂ or CH₃ group within an X³ group, optionally bears on each said CH₂ or CH₃ group one or more halogeno substituents,

and wherein any CH₂ group which is attached to 2 carbon atoms or any CH₃ group which is attached to a carbon atom within a X³ substituent optionally bears on each said CH₂ or CH₃ group a substituent selected from hydroxy and (1-6C)alkoxy;

(ooooooo) X³ is selected from a group of the formula —(CR⁸R⁹)—, —(CR⁸R⁹CH₂)—, —(CR⁸R⁹CH₂CH₂)—, —(CH₂CR⁸R⁹)— and —(CH₂CH₂CR⁸R⁹)—,

each of R⁸ and R⁹, which may be the same or different, is selected from hydrogen and (1-6C)alkyl, provided that at least one of R⁸ or R⁹ in X³ is a branched alkyl group selected from iso-propyl, iso-butyl, sec-butyl and tert-butyl,

and wherein any CH₂ or CH₃ group within an X³ group, optionally bears on each said CH₂ or CH₃ group one or more halogeno substituents,

and wherein any CH₂ group which is attached to 2 carbon atoms or any CH₃ group which is attached to a carbon atom within a X³ substituent optionally bears on each said CH₂ or CH₃ group a substituent selected from hydroxy and (1-6C)alkoxy;

(ppppppp) X³ is selected from a group of the formula —CH₂—, —CH₂CH₂—, —CH₂CH₂CH₂—, —(CR⁸R⁹)—, —(CR⁸R⁹CH₂)— and —(CH₂CR⁸R⁹)—,

wherein each of R⁸ and R⁹, which may be the same or different, is selected from hydrogen, (1-4C)alkyl, hydroxy-(1-4C)alkyl, and (1-3C)alkoxy-(1-4C)alkyl, provided that R⁸ and R⁹ are not both hydrogen;

(qqqqqqq) X³ is selected from a group of the formula —CH₂—, —CH₂CH₂—, —(CHR⁸)—, —(CHR⁸CH₂)— and —(CH₂CHR⁸)—,

wherein R⁸ is selected from hydrogen, (1-4C)alkyl, hydroxy-(1-4C)alkyl and (1-3C)alkoxy-(1-4C)alkyl;

(rrrrrr) X³ is selected from a group of the formula (CH₂)_{q}—, wherein q is 1, 2 or 3, particularly q is 1 or 2;

(sssssss) X³ is —CH₂—;

(ttttttt) Z is selected from hydroxy, amino, (1-6C)alkylamino, di-[(1-6C)alkyl]amino, (1-6C)alkoxy, (1-6C)alkylsulfonyl, (1-6C)alkanesulfonylamino, N-(1-6C)alkyl-(1-6C)alkanesulfonylamino, and a group of the formula:
Q⁴-X⁵—

wherein X⁵ is a direct bond or is selected from O, N(R¹²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein adjacent carbon atoms in any (2-6C)alkylene chain within a Z substituent are optionally separated by the insertion into the chain of a group selected from O, S, SO, SO₂, N(R¹³), CO, —C═C— and —C≡C— wherein R¹³ is hydrogen or (1-6C)alkyl,

and wherein any CH₂ or CH₃ group within a Z group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within a Z substituent optionally bears 1 or 2 oxo or thioxo substituents;

(uuuuuuu) Z is selected from hydroxy, amino, (1-6C)alkylamino, di-[(1-6C)alkyl]amino, (1-6C)alkoxy, (1-6C)alkylsulfonyl, (1-6C)alkanesulfonylamino and N-(1-6C)alkyl-(1-6C)alkanesulfonylamino and a group of the formula:
Q⁴-X⁵—

wherein X⁵ is a direct bond or is selected from O, N(R¹²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein any heterocyclyl group in Z is a monocyclic a fully saturated 4, 5, 6 or 7-membered monocyclic heterocyclyl group containing 1 or 2 heteroatoms selected from oxygen, nitrogen and sulfur,

and wherein any CH₂ or CH₃ group within a Z group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within a Z substituent optionally bears 1 or 2 oxo substituents;

(vvvvvv) Z is selected from hydroxy, amino, (1-6C)alkylamino, di-[(1-6C)alkyl]amino, (1-6C)alkoxy and a group of the formula:
Q⁴-X⁵—

wherein X⁵ is a direct bond or is selected from O and N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein any heterocyclyl group in Z is a monocyclic a non-aromatic fully saturated or partially saturated 4, 5, 6 or 7-membered monocyclic heterocyclyl group containing 1 heteroatom selected from oxygen and nitrogen and optionally a further heteroatom selected from oxygen, nitrogen and sulfur,

and wherein any CH₂ or CH₃ group within a Z group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl;

(wwwwwww) Z is selected from hydroxy, amino, (1-6C)alkylamino, di-[(1-6C)alkyl]amino, (1-6C)alkoxy and a group of the formula:
Q⁴-X⁵—

wherein X⁵ is a direct bond or is selected from O and N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when n, p and q are all 0, then Z is heterocyclyl,

and wherein any heterocyclyl group in Z is selected from tetrahydrofuranyl, 1,3-dioxolanyl, tetrahydropyranyl, 1,4-dioxanyl, oxepanyl, pyrrolidinyl, morpholinyl, tetrahydro-1,4-thiazinyl, piperidinyl, homopiperidinyl, piperazinyl and homopiperazinyl, which heterocyclyl group may be carbon or nitrogen linked to the group to which it is attached,

and wherein any CH₂ or CH₃ group within a Z group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl;

(xxxxxxx) Z is selected from hydroxy, amino, (1-6C)alkylamino, hydroxy-(2-6C)alkylamino, (1-4C)alkoxy-(2-6C)alkylamino, di-[(1-6C)alkyl]amino, N-[hydroxy-(2-6C)alkyl]-N-(1-6C)alkylamino, N-[(1-4C)alkoxy-(2-6C)alkyl]-N-(1-6C)alkylamino, di-[hydroxy-(2-6C)alkyl]-amino, di-[(1-4C)alkoxy-(2-6C)alkyl]amino, N-[(1-4C)alkoxy-(2-6C)alkyl]-N-[hydroxy-(2-6C)alkyl]-amino, (1-6C)alkoxy, hydroxy-(2-6C)alkoxy, (1-4C)alkoxy-(2-6C)alkoxy, azetidin-1-yl, pyrrolidin-1-yl, piperidino, piperazin-1-yl, morpholino, homopiperidin-1-yl homopiperazin-1-yl, tetrahydrofuran-2-yl, tetrahydrofuran-3-yl, 1,3-dioxolanyl, tetrahydropyranyl, 1,4-dioxanyl and a group of the formula:
Q⁴-X⁵—

wherein X⁵ is selected from O and N(R¹²), wherein R¹² is hydrogen or (1-4C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

and provided that when m, p and q are all 0, then Z is heterocyclyl,

and wherein any heterocyclyl group in Z is selected from tetrahydrofuranyl, 1,3-dioxolanyl, tetrahydropyranyl, 1,4-dioxanyl, oxepanyl, pyrrolidinyl, morpholinyl, tetrahydro-1,4-thiazinyl, piperidinyl, homopiperidinyl, piperazinyl, homopiperazinyl, which heterocyclyl group may be carbon or nitrogen linked to the group to which it is attached,

and wherein any CH₂ or CH₃ group within a Z group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(4C)alkyl, N-(1-4C)alkylamino-(1-4C)alkyl and N,N-di-[(1-4C)alkyl]amino-(1-4C)alkyl,

(yyyyyyy) Z is selected from amino, (1-6C)alkylamino, hydroxy-(2-6C)alkylamino, (1-4C)alkoxy-(2-6C)alkylamino, di-[(1-6C)alkyl]amino, N-[hydroxy-(2-6C)alkyl]-N-(1-6C)alkylamino, N-[(1-4C)alkoxy-(2-6C)alkyl]-N-(1-6C)alkylamino, di-[hydroxy-(2-6C)alkyl]-amino, di-[(1-4C)alkoxy-(2-6C)alkyl]amino, N-[(1-4C)alkoxy-(2-6C)alkyl]-N-[hydroxy-(2-6C)alkyl]-amino, azetidin-1-yl, pyrrolidin-1-yl, piperidino, piperazin-1-yl, morpholino, homopiperidin-1-yl and homopiperazin-1-yl,

and wherein and wherein any CH₂ or CH₃ group within a Z group, optionally bears on each said CH₂ or CH₃ group one or more fluoro substituents or a substituent selected from hydroxy, cyano, amino, (2-6C)alkenyl, (2-6C)alkynyl, (1-6C)alkoxy, (1-6C)alkylamino and di-[(1-6C)alkyl]amino,

and wherein any heterocyclyl group within a Z substituent optionally bears one or more (for example 1, 2 or 3) substitutents which may be the same or different, selected from halogeno, cyano, hydroxy, amino, (1-4C)alkyl, (1-4C)alkoxy, (1-4C)alkylamino and di-[(1-4C)alkyl]amino;

(zzzzzzz) Z is selected from hydroxy, (1-6C)alkoxy, hydroxy-(2-6C)alkoxy, (1-4C)alkoxy-(2-6C)alkoxy, tetrahydrofuran-2-yl, tetrahydrofuran-3-yl, 1,3-dioxolanyl, tetrahydropyranyl, 1,4-dioxanyl and a group of the formula:
Q⁴-X⁵—

wherein X⁵ is O, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

and provided that when m, p and q are all 0, then Z is heterocyclyl,

and wherein any heterocyclyl group in Z is selected from tetrahydrofuranyl, 1,3-dioxolanyl, tetrahydropyranyl, 1,4-dioxanyl and oxepanyl,

and wherein any CH₂ or CH₃ group within a Z group, optionally bears on each said CH₂ or CH₃ group one or more fluoro substituents or a substituent selected from hydroxy, cyano, amino, (2-6C)alkenyl, (2-6C)alkynyl, (1-6C)alkoxy, (1-6C)alkylamino and di-[(1-6C)alkyl]amino,

and wherein any heterocyclyl group within a Z substituent optionally bears one or more (for example 1, 2 or 3) substitutents which may be the same or different, selected from halogeno, cyano, hydroxy, amino, (1-4C)alkyl, (1-4C)alkoxy, (1-4C)alkylamino and di-[(1-4C)alkyl]amino;

(aaaaaaaa) Z is selected from hydroxy, amino, (1-6C)alkylamino, hydroxy-(2-6C)alkylamino, (1-4C)alkoxy-(2-6C)alkylamino, di-[(1-6C)alkyl]amino, N-[hydroxy-(2-6C)alkyl]-N-(1-6C)alkylamino, N-[(1-4C)alkoxy-(2-6C)alkyl]-N-(1-6C)alkylamino, di-[hydroxy-(2-6C)alkyl]-amino, di-[(1-4C)alkoxy-(2-6C)alkyl]amino, N-[(1-4C)alkoxy-(2-6C)alkyl]-N-[hydroxy-(2-6C)alkyl]-amino, (1-6C)alkoxy, hydroxy-(2-6C)alkoxy and (1-4C)alkoxy-(2-6C)alkoxy;

(bbbbbbbb) Z is selected from hydroxy, methoxy, ethoxy, 2-hydroxyethoxy, 2-methoxyethoxy, amino, methylamino, ethylamino, N-(2-hydroxyethyl)amino, N-(2-methoxyethyl)amino, dimethylamino, N-methyl-N-ethylamino, di-ethylamino, N-(2-hydroxyethyl)-N-methylamino, N-(2-hydroxyethyl)-N-ethylamino, N,N-di-(2-hydroxyethyl)amino, N-(2-methoxyethyl)-N-methylamino, N-(2-methoxyethyl)-N-ethylamino, pyrrolidin-1-yl, piperidino, piperazin-1-yl, morpholino, tetrahydrofuranyl and tetrahydropyranyl,

and wherein any heterocyclyl group within Z optionally bears 1 or 2 substituents, which may be the same or different, selected from fluoro, chloro, hydroxy, (1-4C)alkyl and (1-4C)alkoxy;

(cccccccc) Z is selected from N-[hydroxy-(2-4C)alkyl]-amino, N-[(1-4C)alkoxy-(2-4C)alkyl]-amino, N-[hydroxy-(2-4C)alkyl]-N-[(1-4C)alkyl]amino, N,N-di-[hydroxy-(2-4C)alkyl]-amino, N-[(1-4C)alkoxy-(2-4C)alkyl]-N-[(1-4C)alkyl]amino and hydroxy-(2-6C)alkoxy and (1-4C)alkoxy-(2-6C)alkoxy;

(dddddddd) Z is selected from pyrrolidin-1-yl, piperidino, piperazin-1-yl, morpholino, homopiperidin-1-yl, homopiperazin-1-yl, (particularly Z is selected from pyrrolidin-1-yl, piperidino, piperazin-1-yl and morpholino),

and wherein the heterocyclyl group within Z optionally bears one or more (for example 1, 2 or 3) substituents, which may be the same or different selected from fluoro, chloro, cyano, hydroxy, amino, carbamoyl, (1-4C)alkyl, (1-4C)alkoxy, (1-4C)alkylamino, di-[(1-4C)alkyl]amino, N-(1-4C)alkylcarbamoyl, N,N-di-[(1-4C)alkyl]carbamoyl, acetyl, propionyl, 2-fluoroethyl, 2-hydroxyethyl, 2-methoxyethyl, cyanomethyl, hydroxyacetyl, aminoacetyl, methylaminoacetyl, ethylaminoacetyl, dimethylaminoacetyl and N-methyl-N-ethylaminoacetyl;

(eeeeeeee) Z is hydroxy;

(ffffffff) Z is selected from hydrogen, hydroxy, amino, (1-6C)alkylamino, di-[(1-6C)alkyl]amino, (1-6C)alkoxy, and a group of the formula:
Q⁴-X⁵—

wherein X⁵ is a direct bond or is selected from O, N(R¹²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein adjacent carbon atoms in any (2-6C)alkylene chain within a Z substituent are optionally separated by the insertion into the chain of a group selected from O, S, SO, SO₂, N(R¹³), CO, —C═C— and —C≡C— wherein R¹³ is hydrogen or (1-6C)alkyl,

and wherein any CH₂ or CH₃ group within a Z group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within a Z substituent optionally bears 1 or 2 oxo or thioxo substituents;

(gggggggg) Z is selected from hydrogen, hydroxy, amino, (1-6C)alkylamino, di-[(1-6C)alkyl]amino and (1-6C)alkoxy, and a group of the formula:
Q⁴-X⁵—

wherein X⁵ is a direct bond or is selected from O, N(R¹²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein any heterocyclyl group in Z is a monocyclic a fully saturated 4, 5, 6 or 7-membered monocyclic heterocyclyl group containing 1 or 2 heteroatoms selected from oxygen, nitrogen and sulfur,

and wherein any CH₂ or CH₃ group within a Z group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group within a Z substituent optionally bears 1 or 2 oxo substituents;

(hhhhhhhh) Z is selected from hydrogen, hydroxy, amino, (1-6C)alkylamino, di-[(1-6C)alkyl]amino and (1-6C)alkoxy and a group of the formula:
Q⁴-X⁵—

wherein X⁵ is a direct bond or is selected from O and N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein any heterocyclyl group in Z is selected from tetrahydrofuranyl, 1,3-dioxolanyl, tetrahydropyranyl, 1,4-dioxanyl, oxepanyl, pyrrolidinyl, morpholinyl, tetrahydro-1,4-thiazinyl, piperidinyl, homopiperidinyl, piperazinyl and homopiperazinyl, which heterocyclyl group may be carbon or nitrogen linked to the group to which it is attached,

and wherein any CH₂ or CH₃ group within a Z group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl;

(iiiiiiii) Z is selected from hydrogen, hydroxy, amino, (1-6C)alkylamino, hydroxy-(2-6C)alkylamino, (1-4C)alkoxy-(2-6C)alkylamino, di-[(1-6C)alkyl]amino, N-[hydroxy-(2-6C)alkyl]-N-(1-6C)alkylamino, N-[(1-4C)alkoxy-(2-6C)alkyl]-N-(1-6C)alkylamino, di-[hydroxy-(2-6C)alkyl]-amino, di-[(1-4C)alkoxy-(2-6C)alkyl]amino, N-[(1-4C)alkoxy-(2-6C)alkyl]-N-[hydroxy-(2-6C)alkyl]-amino, (1-6C)alkoxy, hydroxy-(2-6C)alkoxy, (1-4C)alkoxy-(2-6C)alkoxy, azetidin-1-yl, pyrrolidin-1-yl, piperidino, piperazin-1-yl, morpholino, homopiperidin-1-yl homopiperazin-1-yl, tetrahydrofuran-2-yl, tetrahydrofuran-3-yl, 1,3-dioxolanyl, tetrahydropyranyl, 1,4-dioxanyl and a group of the formula:
Q⁴-X⁵—

wherein X⁵ is selected from O and N(R¹²), wherein R¹² is hydrogen or (1-4C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

and provided that when m, p and q are all 0, then Z is heterocyclyl,

and wherein any heterocyclyl group in Z is selected from tetrahydrofuranyl, 1,3-dioxolanyl, tetrahydropyranyl, 1,4-dioxanyl, oxepanyl, pyrrolidinyl, morpholinyl, tetrahydro-1,4-thiazinyl, piperidinyl, homopiperidinyl, piperazinyl, homopiperazinyl, which heterocyclyl group may be carbon or nitrogen linked to the group to which it is attached,

and wherein any CH₂ or CH₃ group within a Z group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl;

(jjjjjjjj) Z is selected from hydrogen, hydroxy, amino, (1-6C)alkylamino, hydroxy-(2-6C)alkylamino, (1-4C)alkoxy-(2-6C)alkylamino, di-[(1-6C)alkyl]amino, N-[hydroxy-(2-6C)alkyl]-N-(1-6C)alkylamino, N-[(1-4C)alkoxy-(2-6C)alkyl]-N-(1-6C)alkylamino, di-[hydroxy-(2-6C)alkyl]-amino, di-[(1-4C)alkoxy-(2-6C)alkyl]amino, N-[(1-4C)alkoxy-(2-6C)alkyl]-N-[hydroxy-(2-6C)alkyl]-amino, (1-6C)alkoxy, hydroxy-(2-6C)alkoxy and (1-4C)alkoxy-(2-6C)alkoxy;

(kkkkkkkk) Z is selected from hydrogen, hydroxy, methoxy, ethoxy, 2-hydroxyethoxy, 2-methoxyethoxy, amino, methylamino, ethylamino, N-(2-hydroxyethyl)amino, N-(2-methoxyethyl)amino, dimethylamino, N-methyl-N-ethylamino, di-ethylamino, N-(2-hydroxyethyl)-N-methylamino, N-(2-hydroxyethyl)-N-ethylamino, N,N-di-(2-hydroxyethyl)amino, N-(2-methoxyethyl)-N-methylamino, N-(2-methoxyethyl)-N-ethylamino, pyrrolidin-1-yl, piperidino, piperazin-1-yl, morpholino, tetrahydrofuranyl and tetrahydropyranyl,

and wherein any heterocyclyl group within Z optionally bears 1 or 2 substituents, which may be the same or different, selected from fluoro, chloro, hydroxy, (1-4C)alkyl and (1-4C)alkoxy;

(llllllll) Z is hydrogen;

(mmmmmmmm) Z is hydroxy;

(nnnnnnnn) Z is dimethylamino;

(oooooooo) Z is as defined in any of (ttttttt) to (nnnnnnnn) above,

and wherein X³ is selected from —CH₂—, —CH₂CH₂—, —(CR⁸R⁹)—, —(CR⁸R⁹CH₂)—, —(CH₂CR⁸R⁹)— and (3-6C)cycloalkenylene (for example cyclopropylene such as 1,1-cyclopropylene),

wherein each of R⁸ and R⁹, which may be the same or different, is selected from hydrogen, (1-4C)alkyl, hydroxy-(1-4C)alkyl, and (1-3C)alkoxy-(1-4C)alkyl, provided that R⁸ and R⁹ are not both hydrogen,

and M is CO;

(pppppppp) Z is as defined in any of (ttttt) to (nnnnnnnn) above,

and wherein X³ is selected from —CH₂—, —CH₂CH₂—, —(CR⁸R⁹)—, —(CR⁸R⁹CH₂)—, —(CH₂CR⁸R⁹)— and (3-6C)cycloalkenylene (for example cyclopropylene such as 1,1-cyclopropylene),

wherein each of R⁸ and R⁹, which may be the same or different, is selected from hydrogen, (1-4C)alkyl, hydroxy-(1-4C)alkyl, and (1-3C)alkoxy-(1-4C)alkyl, provided that R⁸ and R⁹ are not both hydrogen,

and M is SO₂;

(qqqqqqqq) Z-X³-M is (1-4C)alkylsulfonyl, for example methylsulfonyl;

(rrrrrrrr) Z-X³-M is (2-4C)alkanoyl, for example acetyl;

(ssssssss) Z-X³-M is hydroxy-(2-4C)alkanoyl, for example hydroxyacetyl;

(tttttttt) Z-X³-M is di[(1-6C)alkyl]amino-(2-4C)alkanoyl, for example (dimethylamino)acetyl;

(uuuuuuuu) Z-X³-M is selected from methylsulfonyl, acetyl, hydroxyacetyl and (dimethylamino)acetyl;

(vvvvvv) Z-X³— is selected from tetrahydrofuranyl, 1,3-dioxolanyl, tetrahydropyranyl, 1,4-dioxanyl, oxepanyl, pyrrolidinyl, morpholinyl, piperidinyl, homopiperidinyl, piperazinyl and homopiperazinyl, which heterocyclyl is linked to the carbonyl group in Formula I, by a ring carbon,

and wherein the heterocyclyl group within Z-X³ optionally bears 1 or 2 substituents, which may be the same or different, selected from fluoro, chloro, hydroxy, (1-4C)alkyl, (1-4C)alkoxy and (2-4C)alkanoyl,

and M is CO; and

(wwwwwwww) Z-X³— is selected from tetrahydrofuranyl, 1,3-dioxolanyl, tetrahydropyranyl, 1,4-dioxanyl, oxepanyl (for example Z-X³ is selected tetrahydrofuran-2-yl or tetrahydropyran-2-yl),

and M is CO.

A particular embodiment of the present invention is a quinazoline derivative of the formula I wherein:

R¹ is selected from hydrogen and (1-3C)alkoxy, (for example R¹ is hydrogen or methoxy, particularly hydrogen);

X¹ is a direct bond or CH₂;

X² is selected from O, S and OCH₂;

Q² is heteroaryl or phenyl,

and wherein Q² optionally bears one or more substituents (for example 1, 2 or 3), which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, and a group of the formula:
—X⁴—R⁵

wherein X⁴ is a direct bond or is selected from O, CO and N(R⁶), wherein R⁶ is hydrogen or (1-6C)alkyl, and R⁵ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulfamoyl(1-6C)alkyl, N-(1-6C)alkylsulfamoyl(1-6C)alkyl, N di-(1-6C)alkylsulfamoyl(1-6C)alkyl, (2-6C)alkanoyl-(1-6C)alkyl, (2-6C)alkanoyloxy-(1-6C)alkyl or (1-6C)alkoxycarbonyl-(1-6C)alkyl,

and wherein any CH₂ or CH₃ group within Q² optionally bears on each said CH₂ or CH₃ one or more (for example 1, 2, or 3) halogeno or (1-6C)alkyl substituents or a substituent selected from hydroxy, cyano, amino, (1-4C)alkoxy, (1-4C)alkylamino and di-[(1-4C)alkylamino];

M is CO;

and wherein R², Y, Q¹, X³, a and Z have any of the values defined hereinbefore;

or a pharmaceutically acceptable salt thereof.

In this embodiment a particular value for Q² is a 5 or 6 membered heteroaryl ring containing 1 nitrogen heteroatom and optionally 1 additional heteroatom selected from O, S and N, and wherein Q² optionally bears one or more substituents as defined above.

In this embodiment a particular value for X² is OCH₂.

In this embodiment a particular value for a is 0 or 1, more particularly 0.

In this embodiment Z is preferably not hydrogen.

Another embodiment of the present invention is a quinazoline derivative of the Formula I wherein:

R¹ is selected from hydrogen and (1-3C)alkoxy, (for example R¹ is hydrogen or methoxy, particularly hydrogen);

Y is selected from halogeno (particularly chloro), (1-4C)alkyl, (1-4C)alkoxy and (2-4C)alkynyl;

a is 0 or 1;

R² is halogeno;

X² is selected from O, S and OCH₂;

Q² is selected from phenyl and a 5 or 6 membered heteroaryl ring containing 1 nitrogen heteroatom and optionally 1 additional heteroatom selected from O, S and N,

and wherein Q² optionally bears one or more substituents (for example 1, 2 or 3), which may be the same or different selected from halogeno, hydroxy, cyano, carboxy, nitro, amino, (1-4C)alkyl, (1-4C)alkoxy, (2-4C)alkenyl, (2-4C)alkynyl, (1-4C)alkylthio, (1-4C)alkylsulfinyl, (1-4C)alkylsulfonyl, (2-4C)alkanoyl, N-(1-4C)alkylamino, N. N-di-[(1-4C)alkyl]amino, (1-4C)alkoxycarbonyl, carbamoyl, N-(1-4C)alkylcarbamoyl, N,N-di-[(1-4C)alkyl]carbamoyl, (2-4C)alkanoyloxy, (2-4C)alkanoylamino, N-(1-4C)alkyl-(2-4C)alkanoylamino, halogeno-(1-4C)alkyl, hydroxy-(1-4C)alkyl, (1-4C)alkoxy-(1-4C)alkyl, cyano-(1-4C)alkyl, carboxy-(1-4C)alkyl, amino-(1-4C)alkyl, N-(1-4C)alkylamino-(1-4C)alkyl and N,N-di-[(1-4C)alkyl]amino-(1-4C)alkyl;

X¹ is a direct bond or CH₂;

Q¹ is selected from pyrrolidinyl and piperidinyl

and wherein Q¹ optionally bears 1 or 2 substituents, which may be the same or different, selected from hydroxy, (1-4C)alkyl and (1-4C)alkoxy,

and wherein Q¹ optionally bears an oxo substituent,

and wherein Q¹ is linked to the group X¹ by a ring carbon;

M is CO;

X³ is selected from CH₂—, —CH₂CH₂—, —(CR⁸R⁹)—, —(CR⁸R⁹CH₂)—, —(CH₂CR⁸R⁹)— and (3-6C)cycloalkylene (for example cyclopropylene such as cyclopropylidene),

wherein each of R⁸ and R⁹, which may be the same or different, is selected from hydrogen, (1-4C)alkyl, hydroxy-(1-4C)alkyl, and (1-3C)alkoxy-(1-4C)alkyl, provided that R⁸ and R⁹ are not both hydrogen;

Z is selected from hydroxy, amino, (1-6C)alkylamino, hydroxy-(2-6C)alkylamino, (1-4C)alkoxy-(2-6C)alkylamino, di-[(1-6C)alkyl]amino, N-[hydroxy-(2-6C)alkyl]-N-(1-6C)alkylamino, N-[(1-4C)alkoxy-(2-6C)alkyl]-N-(1-6C)alkylamino, di-[hydroxy-(2-6C)alkyl]-amino, di-[(1-4C)alkoxy-(2-6C)alkyl]amino, N-[(1-4C)alkoxy-(2-6C)alkyl]-N-[hydroxy-(2-6C)alkyl]-amino, (1-6C)alkoxy, hydroxy-(2-6C)alkoxy and (1-4C)alkoxy-(2-6C)alkoxy;

or a pharmaceutically acceptable salt thereof.

In this embodiment a particular value for X¹ is CH₂ and Q¹ is selected from pyrrolidin-2-yl, pyrrolidin-3-yl, piperidin-3-yl and piperidin-4-yl. Still more particularly in this embodiment X¹ is CH₂; Q¹ is selected from pyrrolidin-2-yl, pyrrolidin-3-yl, piperidin-3-yl and piperidin-4-yl; and Z-X³ is selected from hydroxymethyl, aminomethyl, (1-4C)alkylaminomethyl and di-[(1-4C)alkyl]aminomethyl (more particularly Z-X³ is hydroxymethyl or di-methylaminomethyl, still more particularly Z-X³ is hydroxymethyl).

In this embodiment a particular value for Q² is pyridyl, pyrazinyl, 1,3-thiazolyl or isoxazolyl, more particularly Q² is selected from 2-pyridyl and 2-pyrazinyl,

and wherein Q² optionally bears one or more substituents as defined above for this embodiment.

Another embodiment of the present invention is a quinazoline derivative of the Formula I wherein:

R¹ is selected from hydrogen and (1-3C)alkoxy, (for example R¹ is hydrogen or methoxy, particularly hydrogen);

Y is selected from hydrogen, halogeno and (1-4C)alkoxy;

a is 0 or 1;

R² is halogeno;

X² is OCH₂;

Q² is phenyl which optionally bears 1 or 2 halogeno (particularly fluoro) substituents;

X¹ is a direct bond or CH₂;

Q¹ is selected from pyrrolidinyl and piperidinyl,

and wherein Q¹ optionally bears 1 or 2 substituents, which may be the same or different, selected from hydroxy, (1-4C)alkyl and (1-4C)alkoxy,

and wherein Q¹ optionally bears an oxo substituent,

and wherein Q¹ is linked to the group X¹ by a ring carbon;

M is CO;

X³ is selected from —CH₂—, —CH₂CH₂—, —(CR⁸R⁹)—, —(CR⁸R⁹CH₂)—, —(CH₂CR⁸R⁹)— and (3-6C)cycloalkylene (for example cyclopropylene such as cyclopropylidene),

wherein each of R⁸ and R⁹, which may be the same or different, is selected from hydrogen, (1-4C)alkyl, hydroxy-(1-4C)alkyl, and (1-3C)alkoxy-(1-4C)alkyl, provided that R⁸ and R⁹ are not both hydrogen;

Z is selected from hydroxy, amino, (1-6C)alkylamino, hydroxy-(2-6C)alkylamino, (1-4C)alkoxy-(2-6C)alkylamino, di-[(1-6C)alkyl]amino, N-[hydroxy-(2-6C)alkyl]-N-(1-6C)alkylamino, N-[(1-4C)alkoxy-(2-6C)alkyl]-N-(1-6C)alkylamino, di-[hydroxy-(2-6C)alkyl]-amino, di-[(1-4C)alkoxy-(2-6C)alkyl]amino, N-[(1-4C)alkoxy-(2-6C)alkyl]-N-[hydroxy-(2-6C)alkyl]-amino, (1-6C)alkoxy, hydroxy-(2-6C)alkoxy and (1-4C)alkoxy-(2-6C)alkoxy;

or a pharmaceutically acceptable salt thereof.

In this embodiment a particular value for X¹ is CH₂ and Q¹ is selected from pyrrolidin-2-yl, pyrrolidin-3-yl, piperidin-3-yl and piperidin-4-yl. Still more particularly in this embodiment X¹ is CH₂; Q¹ is selected from pyrrolidin-2-yl, pyrrolidin-3-yl, piperidin-3-yl and piperidin-4-yl; and Z-X³ is selected from hydroxymethyl, aminomethyl, (1-4C)alkylaminomethyl and di-[(1-4C)alkyl]aminomethyl (more particularly Z-X³ is hydroxymethyl or di-methylaminomethyl, still more particularly Z-X³ is hydroxymethyl).

In this embodiment a particular value for Q² is phenyl substituted by 1 or 2 substituents, which may be the same or different, selected from fluoro and chloro, for example Q² is 3-fluorophenyl; a is 0; and Y is selected from hydrogen, chloro and methoxy (particularly Y is methoxy).

Another embodiment of the present invention is a quinazoline derivative of the Formula I wherein:

R¹ is selected from hydrogen and (1-3C)alkoxy, (for example R¹ is hydrogen or methoxy, particularly hydrogen);

Y is selected halogeno particularly chloro);

a is 0 or 1;

R² is halogeno;

X² is OCH₂;

Q² is selected from 2-pyridyl and 2-pyrazinyl which optionally bears 1 or 2 substituents, which may be the same or different, selected from (1-3C)alkyl, (1-3-C)alkoxy and halogeno (particularly fluoro);

X¹ is a direct bond or CH₂;

Q¹ is selected from pyrrolidinyl and piperidinyl,

and wherein Q¹ optionally bears 1 or 2 substituents, which may be the same or different, selected from hydroxy, (1-4C)alkyl and (1-4C)alkoxy,

and wherein Q¹ optionally bears an oxo substituent,

and wherein Q¹ is linked to the group X¹ by a ring carbon;

M is CO;

X³ is selected from —CH₂—, —CH₂CH₂—, —(CR⁸R⁹)—, —(CR⁸R⁹CH₂)—, —(CH₂CR⁸R⁹)— and (3-6C)cycloalkylene (for example cyclopropylene such as cyclopropylidene),

wherein each of R⁸ and R⁹, which may be the same or different, is selected from hydrogen, (1-4C)alkyl, hydroxy-(1-4C)alkyl, and (1-3C)alkoxy-(1-4C)alkyl, provided that R⁸ and R⁹ are not both hydrogen; and

Z is selected from hydroxy, amino, (1-6C)alkylamino, hydroxy-(2-6C)alkylamino, (1-4C)alkoxy-(2-6C)alkylamino, di-[(1-6C)alkyl]amino, N-[hydroxy-(2-6C)alkyl]-N-(1-6C)alkylamino, N-[(1-4C)alkoxy-(2-6C)alkyl]-N-(1-6C)alkylamino, di-[hydroxy-(2-6C)alkyl]-amino, di-[(1-4C)alkoxy-(2-6C)alkyl]amino, N-[(1-4C)alkoxy-(2-6C)alkyl]-N-[hydroxy-(2-6C)alkyl]-amino, (1-6C)alkoxy, hydroxy-(2-6C)alkoxy and (1-4C)alkoxy-(2-6C)alkoxy;

or a pharmaceutically acceptable salt thereof.

In this embodiment a particular value for X¹ is CH₂ and Q¹ is selected from pyrrolidin-2-yl, pyrrolidin-3-yl, piperidin-3-yl and piperidin-4-yl. Still more particularly in this embodiment X¹ is CH₂; Q¹ is selected from pyrrolidin-2-yl, pyrrolidin-3-yl, piperidin-3-yl and piperidin-4-yl; and Z-X³ is selected from hydroxymethyl, aminomethyl, (1-4C)alkylaminomethyl and di-[(1-4C)alkyl]aminomethyl (more particularly Z-X³ is hydroxymethyl or di-methylaminomethyl, still more particularly Z-X³ is hydroxymethyl).

In this embodiment a particular value for Q² is 2-pyridyl or 2-pyrazinyl; a is 0; and Y is chloro.

Another embodiment of the compounds of Formula I is a quinazoline derivative of the Formula IA: 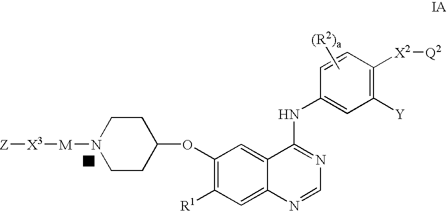
wherein:

R¹ is selected from hydrogen, hydroxy, (1-6C)alkoxy, (3-7C)cycloalkyl-oxy and (3-7C)cycloalkyl-(1-6C)alkoxy,

and wherein adjacent carbon atoms in any (2-6C)alkylene chain within a R¹ substituent are optionally separated by the insertion into the chain of a group selected from O, S, SO, SO₂, N(R³), CO, CON(R³), N(R³)CO, SO₂N(R³) and N(R³)SO₂, wherein R³ is hydrogen or (1-6C)alkyl,

and wherein any CH₂ or CH₃ group within a R¹ substituent optionally bears on each said CH₂ or CH₃ group one or more halogeno or (1-6C)alkyl substituents, or a substituent selected from hydroxy, cyano, amino, carboxy, carbamoyl, sulfamoyl, oxo, thioxo, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino;

Y is selected from hydrogen, halogeno, (1-4C)alkyl, (1-4C)alkoxy, (2-4C)alkenyl and (2-4C)alkenyl;

a is 0, 1, 2 or 3 or 4;

each R², which may be the same or different, is selected from halogeno, (1-4C)alkyl, (1-4C)alkoxy, (2-4C)alkenyl and (2-4C)alkynyl;

X² is a direct bond or is selected from O, S, OC(R⁴)₂, SC(R⁴)₂, SO, SO₂, N(R⁴), CO and N(R⁴)C(R⁴)₂ wherein each R⁴ is, which may be the same or different, is selected from hydrogen or (1-6C)alkyl, and Q² is aryl or heteroaryl,

and wherein Q² optionally bears one or more substituents (for example 1, 2 or 3), which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N E-di-[(1-6C)alkyl]sulfamoyl, (1-6C)alkanesulfonylamino, N-(1-6C)alkyl-(1-6C)alkanesulfonylamino, and a group of the formula:
—X⁴—R⁵

wherein X⁴ is a direct bond or is selected from O, CO and N(R⁶), wherein R⁶ is hydrogen or (1-6C)alkyl, and R⁵ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulfamoyl(1-6C)alkyl, N-(1-6C)alkylsulfamoyl(1-6C)alkyl, N,N di-(1-6C)alkylsulfamoyl(1-6C)alkyl, (2-6C)alkanoyl-(1-6C)alkyl, (2-6C)alkanoyloxy-(1-6C)alkyl or (1-6C)alkoxycarbonyl-(1-6C)alkyl,

and wherein any CH₂ or CH₃ group within —X²-Q² optionally bears on each said CH₂ or CH₃ one or more (for example 1, 2, or 3) halogeno or (1-6C)alkyl substituents or a substituent selected from hydroxy, cyano, amino, (1-4C)alkoxy, (1-4C)alkylamino and di-[(1-4C)alkylamino];

M is selected from CO and SO₂;

X³ is a group of the formula:
—(CR⁸R⁹)ₚ-(Q³)ₘ-(CR¹⁰R¹¹)_{q}—
wherein m is 0 or 1, p is 0, 1, 2, 3 or 4 and q is 0, 1, 2, 3 or 4,

each of R⁸, R⁹, R¹⁰ and R¹¹, which may be the same or different, is selected from hydrogen and (1-6C)alkyl, and

Q³ is selected from (3-7C)cycloalkylene and (3-7C)cycloalkenylene;

Z is selected from hydrogen, hydroxy, amino, (1-6C)alkylamino, di-[(1-6C)alkyl]amino, (1-6C)alkoxy, (1-6C)alkylsulfonyl, (1-6C)alkanesulfonylamino, N-(1-6C)alkyl-(1-6C)alkanesulfonylamino, and a group of the formula:
Q⁴-X⁵—

wherein X⁵ is a direct bond or is selected from O, N(R¹²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein adjacent carbon atoms in any (2-6C)alkylene chain within a Z substituent are optionally separated by the insertion into the chain of a group selected from O, S, SO, SO₂, N(R¹³), CO, —C═C— and —C≡C— wherein R¹³ is hydrogen or (1-6C)alkyl,

and wherein any CH₂ or CH₃ group within any Z, X¹ or X³ group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group represented by Q¹ or within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group represented by Q¹ or within a Z substituent optionally bears 1 or 2 oxo or thioxo substituents;

or a pharmaceutically acceptable salt thereof.

In this embodiment a particular value for R¹ is hydrogen, hydroxy or (1-6C)alkoxy, more particularly hydrogen or (1-3C)alkoxy (such as methoxy).

In this embodiment a particular value for Y is hydrogen, halogeno, (1-4C)alkyl, (1-4C)alkoxy or (2-4C)alkynyl. More particularly, Y is selected from hydrogen, chloro, fluoro, methyl, methoxy and ethynyl.

In this embodiment a particular value for a is 0 or 1, more particularly 0.

In this embodiment a particular value for X² is O, S or OC(R⁴)₂ wherein each R⁴ is, independently, hydrogen or (1-4C)alkyl. More particularly, X² is selected from O, S and OCH₂.

In this embodiment a particular value for Q² is (optionally substituted) phenyl or a 5- or 6-membered monocyclic heteroaryl ring, which ring contains 1 nitrogen heteroatom and optionally 1 or 2 (particularly 1) additional heteroatom independently selected from oxygen, nitrogen and sulfur. More particularly Q² is selected from phenyl, pyridyl, pyrazinyl, 1,3-thiazolyl and 1H-imidazolyl (for example 2-pyridyl, 6-methyl-pyrid-3yl, 3-fluorophenyl, 2-pyrazinyl, 1,3-thiazol-2-yl and 1-methyl-1H-imidazol-2-yl).

In this embodiment a particular value for X³ is a group of the formula —(CR⁸R⁹)ₚ, wherein p is 0, 1 or 2 and each of R⁸ and R⁹, which may be the same or different, is selected from hydrogen and (1-4C)alkyl. For example, a particular value for X³ is —CH₂—.

In this embodiment a particular value for Z is hydrogen, hydroxy, amino, (1-6C)alkylamino or di-[(1-6C)alkyl]amino. More particularly Z is selected from hydrogen, hydroxy and dimethylamino.

Another embodiment of the compounds of Formula I is a quinazoline derivative of the Formula IB: 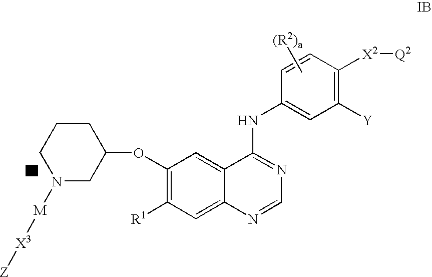
wherein:

R¹ is selected from hydrogen, hydroxy, (1-6C)alkoxy, (3-7C)cycloalkyl-oxy and (3-7C)cycloalkyl-(1-6C)alkoxy,

and wherein adjacent carbon atoms in any (2-6C)alkylene chain within a R¹ substituent are optionally separated by the insertion into the chain of a group selected from O, S, SO, SO₂, N(R³), CO, CON(R³), N(R³)CO, SO₂N(R³) and N(R³)SO₂, wherein R³ is hydrogen or (1-6C)alkyl,

and wherein any CH₂ or CH₃ group within a R¹ substituent optionally bears on each said CH₂ or CH₃ group one or more halogeno or (1-6C)alkyl substituents, or a substituent selected from hydroxy, cyano, amino, carboxy, carbamoyl, sulfamoyl, oxo, thioxo, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino;

Y is selected from hydrogen, halogeno, (1-4C)alkyl, (1-4C)alkoxy, (2-4C)alkenyl and (2-4C)alkynyl;

a is 0, 1, 2 or 3 or 4;

each R², which may be the same or different, is selected from halogeno, (1-4C)alkyl, (1-4C)alkoxy, (2-4C)alkenyl and (2-4C)alkynyl;

X² is a direct bond or is selected from O, S, OC(R⁴)₂, SC(R⁴)₂, SO, SO₂, N(R⁴), CO and N(R⁴)C(R⁴)₂ wherein each R⁴ is, which may be the same or different, is selected from hydrogen or (1-6C)alkyl, and Q² is aryl or heteroaryl,

and wherein Q² optionally bears one or more substituents (for example 1, 2 or 3), which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, and a group of the formula:
—X⁴—R⁵

wherein X⁴ is a direct bond or is selected from O, CO and N(R⁶), wherein R⁶ is hydrogen or (1-6C)alkyl, and R⁵ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulfamoyl(1-6C)alkyl, N-(1-6C)alkylsulfamoyl(1-6C)alkyl, N,N di-(1-6C)alkylsulfamoyl(1-6C)alkyl, (2-6C)alkanoyl-(1-6C)alkyl, (2-6C)alkanoyloxy-(1-6C)alkyl or (1-6C)alkoxycarbonyl-(1-6C)alkyl,

and wherein any CH₂ or CH₃ group within —X²-Q² optionally bears on each said CH₂ or CH₃ one or more (for example 1, 2, or 3) halogeno or (1-6C)alkyl substituents or a substituent selected from hydroxy, cyano, amino, (1-4C)alkoxy, (1-4C)alkylamino and di-[(1-4C)alkylamino];

M is selected from CO and SO₂;

X³ is a group of the formula:
—(CR⁸R⁹)ₚ-(Q³)ₘ-(CR¹⁰R¹¹)_{q}—

wherein m is 0 or 1, p is 0, 1, 2, 3 or 4 and q is 0, 1, 2, 3 or 4,

each of R⁸, R⁹, R¹⁰ and R¹¹, which may be the same or different, is selected from hydrogen and (1-6C)alkyl, and

Q³ is selected from (3-7C)cycloalkylene and (3-7C)cycloalkenylene;

Z is selected from hydrogen, hydroxy, amino, (1-6C)alkylamino, di-[(1-6C)alkyl]amino, (1-6C)alkoxy, (1-6C)alkylsulfonyl, (1-6C)alkanesulfonylamino, N-(1-6C)alkyl-(1-6C)alkanesulfonylamino, and a group of the formula:
Q⁴-X⁵—

wherein X⁵ is a direct bond or is selected from O, N(R¹²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein adjacent carbon atoms in any (2-6C)alkylene chain within a Z substituent are optionally separated by the insertion into the chain of a group selected from O, S, SO, SO₂, N(R¹³), CO, —C═C— and —C≡C— wherein R¹³ is hydrogen or (1-6C)alkyl,

and wherein any CH₂ or CH₃ group within any Z, X¹ or X³ group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group represented by Q¹ or within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group represented by Q¹ or within a Z substituent optionally bears 1 or 2 oxo or thioxo substituents;

or a pharmaceutically acceptable salt thereof.

In this embodiment a particular value for R¹ is hydrogen, hydroxy or (1-6C)alkoxy, more particularly hydrogen.

In this embodiment a particular value for Y is hydrogen, halogeno, (1-4C)alkyl, (1-4C)alkoxy or (2-4C)alkynyl. More particularly, Y is selected from halogeno, such as chloro.

In this embodiment a particular value for a is 0.

In this embodiment a particular value for X² is O, S or OC(R⁴)₂ wherein each R⁴ is, independently, hydrogen or (1-4C)alkyl. More particularly, X² is selected from OC(R⁴)₂ wherein each R⁴ is, independently, hydrogen or (1-2C)alkyl, for example X² is OCH₂.

In this embodiment a particular value for Q² is an optionally substituted 5- or 6-membered monocyclic heteroaryl ring, which ring contains 1 nitrogen heteroatom and optionally 1 or 2 (particularly 1) additional heteroatom independently selected from oxygen, nitrogen and sulfur. More particularly Q² is selected from pyridyl, pyrazinyl, 1,3-thiazolyl and 1H-imidazolyl (for example 2-pyridyl, 6-methyl-pyrid-3yl, 3-fluorophenyl, 2-pyrazinyl, 1,3-thiazol-2-yl and 1-methyl-1H-imidazol-2-yl, particularly 2-pyridyl).

In this embodiment, a particular value for M is CO.

In this embodiment a particular value for X³ is a group of the formula —(CR⁸R⁹)ₚ, wherein p is 0, 1 or 2 and each of R⁸ and R⁹, which may be the same or different, is selected from hydrogen and (1-4C)alkyl. For example, a particular value for X³ is —CH₂—.

In this embodiment a particular value for Z is hydrogen, hydroxy, amino, (1-6C)alkylamino or di-[(1-6C)alkyl]amino. More particularly Z is selected from hydroxy and dimethylamino.

Another embodiment of the compounds of Formula I is a quinazoline derivative of the Formula IC: 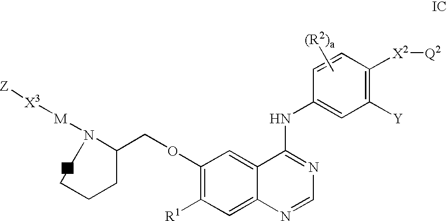
wherein:

R¹ is selected from hydrogen, hydroxy, (1-6C)alkoxy, (3-7C)cycloalkyl-oxy and (3-7C)cycloalkyl-(1-6C)alkoxy,

and wherein adjacent carbon atoms in any (2-6C)alkylene chain within a R¹ substituent are optionally separated by the insertion into the chain of a group selected from O, S, SO, SO₂, N(R³), CO, CON(R³), N(R³)CO, SO₂N(R³) and N(R³)SO₂, wherein R³ is hydrogen or (1-6C)alkyl,

and wherein any CH₂ or CH₃ group within a R¹ substituent optionally bears on each said CH₂ or CH₃ group one or more halogeno or (1-6C)alkyl substituents, or a substituent selected from hydroxy, cyano, amino, carboxy, carbamoyl, sulfamoyl, oxo, thioxo, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino;

Y is selected from hydrogen, halogeno, (1-4C)alkyl, (1-4C)alkoxy, (2-4C)alkenyl and (2-4C)alkynyl;

a is 0, 1, 2 or 3 or 4;

each R², which may be the same or different, is selected from halogeno, (1-4C)alkyl, (1-4C)alkoxy, (2-4C)alkenyl and (2-4C)alkynyl;

X² is a direct bond or is selected from O, S, OC(R⁴)₂, SC(R⁴)₂, SO, SO₂, N(R⁴), CO and N(R⁴)C(R⁴)₂ wherein each R⁴ is, which may be the same or different, is selected from hydrogen or (1-6C)alkyl, and Q² is aryl or heteroaryl,

and wherein Q² optionally bears one or more substituents (for example 1, 2 or 3), which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, and a group of the formula:
—X⁴—R⁵

wherein X⁴ is a direct bond or is selected from O, CO and N(R⁶), wherein R⁶ is hydrogen or (1-6C)alkyl, and R⁵ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulfamoyl(1-6C)alkyl, N-(1-6C)alkylsulfamoyl(1-6C)alkyl, N,N di-(1-6C)alkylsulfamoyl(1-6C)alkyl, (2-6C)alkanoyl-(1-6C)alkyl, (2-6C)alkanoyloxy-(1-6C)alkyl or (1-6C)alkoxycarbonyl-(1-6C)alkyl,

and wherein any CH₂ or CH₃ group within —X²-Q² optionally bears on each said CH₂ or CH₃ one or more (for example 1, 2, or 3) halogeno or (1-6C)alkyl substituents or a substituent selected from hydroxy, cyano, amino, (1-4C)alkoxy, (1-4C)alkylamino and di-[(1-4C)alkylamino];

M is selected from CO and SO₂;

X³ is a group of the formula:
—(CR⁸R⁹)ₚ-(Q³)ₘ-(CR¹⁰R¹¹)_{q}—

wherein m is 0 or 1, p is 0, 1, 2, 3 or 4 and q is 0, 1, 2, 3 or 4,

each of R⁸, R⁹, R¹⁰ and R¹¹, which may be the same or different, is selected from hydrogen and (1-6C)alkyl, and

Q³ is selected from (3-7C)cycloalkylene and (3-7C)cycloalkenylene;

Z is selected from hydrogen, hydroxy, amino, (1-6C)alkylamino, di-[(1-6C)alkyl]amino, (1-6C)alkoxy, (1-6C)alkylsulfonyl, (1-6C)alkanesulfonylamino, N-(1-6C)alkyl-(1-6C)alkanesulfonylamino, and a group of the formula:
Q⁴-X⁵—

wherein X⁵ is a direct bond or is selected from O, N(R¹²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein adjacent carbon atoms in any (2-6C)alkylene chain within a Z substituent are optionally separated by the insertion into the chain of a group selected from O, S, SO, SO₂, N(R¹³), CO, —C═C— and —C≡C— wherein R¹³ is hydrogen or (1-6C)alkyl,

and wherein any CH₂ or CH₃ group within any Z, X¹ or X³ group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group represented by Q¹ or within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group represented by Q¹ or within a Z substituent optionally bears 1 or 2 oxo or thioxo substituents;

or a pharmaceutically acceptable salt thereof.

In this embodiment a particular value for R¹ is hydrogen, hydroxy or (1-6C)alkoxy, more particularly hydrogen.

In this embodiment a particular value for Y is halogeno, for example chloro.

In this embodiment a particular value for a is 0.

In this embodiment a particular value for X² is OC(R⁴)₂ wherein each R⁴ is, independently, hydrogen or (1-4C)alkyl. More particularly, X² is OCH₂.

In this embodiment a particular value for Q² is an optionally substituted 5- or 6-membered monocyclic heteroaryl ring, which ring contains 1 nitrogen heteroatom and optionally 1 or 2 (particularly 1) additional heteroatom independently selected from oxygen, nitrogen and sulfur. More particularly Q² is selected from pyridyl, pyrazinyl, 1,3-thiazolyl and 1H-imidazolyl (for example 2-pyridyl, 6-methyl-pyrid-3yl, 3-fluorophenyl, 2-pyrazinyl, 1,3-thiazol-2-yl and 1-methyl-1H-imidazol-2-yl, particularly 2-pyridyl).

In this embodiment, a particular value for M is CO.

In this embodiment a particular value for X³ is a group of the formula —(CR⁸R⁹)ₚ, wherein p is 0, 1 or 2 and each of R⁸ and R⁹, which may be the same or different, is selected from hydrogen and (1-4C)alkyl. For example, a particular value for X³ is —CH₂—.

In this embodiment a particular value for Z is hydrogen, hydroxy, amino, (1-6C)alkylamino or di-[(1-6C)alkyl]amino. More particularly Z is selected from hydroxy and dimethylamino.

Another embodiment of the compounds of Formula I is a quinazoline derivative of the Formula ID: 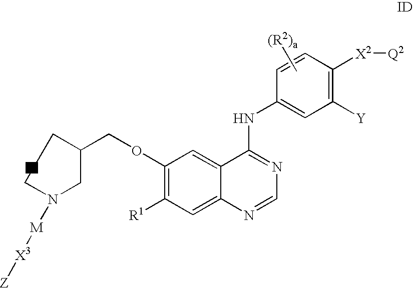
wherein:

R¹ is selected from hydrogen, hydroxy, (1-6C)alkoxy, (3-7C)cycloalkyl-oxy and (3-7C)cycloalkyl-(1-6C)alkoxy,

and wherein adjacent carbon atoms in any (2-6C)alkylene chain within a R¹ substituent are optionally separated by the insertion into the chain of a group selected from O, S, SO, SO₂, N(R³), CO, CON(R³), N(R³)CO, SO₂N(R³) and N(R³)SO₂, wherein R³ is hydrogen or (1-6C)alkyl,

and wherein any CH₂ or CH₃ group within a R¹ substituent optionally bears on each said CH₂ or CH₃ group one or more halogeno or (1-6C)alkyl substituents, or a substituent selected from hydroxy, cyano, amino, carboxy, carbamoyl, sulfamoyl, oxo, thioxo, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino;

Y is selected from hydrogen, halogeno, (1-4C)alkyl, (1-4C)alkoxy, (2-4C)alkenyl and (2-4C)alkynyl;

a is 0, 1, 2 or 3 or 4;

each R², which may be the same or different, is selected from halogeno, (1-4C)alkyl, (1-4C)alkoxy, (2-4C)alkenyl and (2-4C)alkynyl;

X² is a direct bond or is selected from O, S, OC(R⁴)₂, SC(R⁴)₂, SO, SO₂, N(R⁴), CO and N(R⁴)C(R⁴)₂ wherein each R⁴ is, which may be the same or different, is selected from hydrogen or (1-6C)alkyl, and Q² is aryl or heteroaryl,

and wherein Q² optionally bears one or more substituents (for example 1, 2 or 3), which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, and a group of the formula:
—X⁴—R⁵

wherein X⁴ is a direct bond or is selected from O, CO and N(R⁶), wherein R⁶ is hydrogen or (1-6C)alkyl, and R⁵ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulfamoyl(1-6C)alkyl, N-(1-6C)alkylsulfamoyl(1-6C)alkyl, N,N di-(1-6C)alkylsulfamoyl(1-6C)alkyl, (2-6C)alkanoyl-(1-6C)alkyl, (2-6C)alkanoyloxy-(1-6C)alkyl or (1-6C)alkoxycarbonyl-(1-6C)alkyl,

and wherein any CH₂ or CH₃ group within —X²-Q² optionally bears on each said CH₂ or CH₃ one or more (for example 1, 2, or 3) halogeno or (1-6C)alkyl substituents or a substituent selected from hydroxy, cyano, amino, (1-4C)alkoxy, (1-4C)alkylamino and di-[(1-4C)alkylamino];

M is selected from CO and SO₂;

X³ is a group of the formula:
—(CR⁸R⁹)ₚ-(Q³)ₘ-(CR¹⁰R¹¹)_{q}—

wherein m is 0 or 1, p is 0, 1, 2, 3 or 4 and q is 0, 1, 2, 3 or 4,

each of R⁸, R⁹, R¹⁰ and R¹¹, which may be the same or different, is selected from hydrogen and (1-6C)alkyl, and

Q³ is selected from (3-7C)cycloalkylene and (3-7C)cycloalkenylene;

Z is selected from hydrogen, hydroxy, amino, (1-6C)alkylamino, di-[(1-6C)alkyl]amino, (1-6C)alkoxy, (1-6C)alkylsulfonyl, (1-6C)alkanesulfonylamino, N-(1-6C)alkyl-(1-6C)alkanesulfonylamino, and a group of the formula:
Q⁴-X⁵—

wherein X⁵ is a direct bond or is selected from O, N(R¹²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein adjacent carbon atoms in any (2-6C)alkylene chain within a Z substituent re optionally separated by the insertion into the chain of a group selected from O, S, SO, SO₂, N(R¹³), CO, —C═C— and —C≡C— wherein R¹³ is hydrogen or (1-6C)alkyl,

and wherein any CH₂ or CH₃ group within any Z, X¹ or X³ group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group represented by Q¹ or within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group represented by Q¹ or within a Z substituent optionally bears 1 or 2 oxo or thioxo substituents;

or a pharmaceutically acceptable salt thereof.

In this embodiment a particular value for R¹ is hydrogen, hydroxy or (1-6C)alkoxy, more particularly hydrogen.

In this embodiment a particular value for Y is halogeno, for example chloro.

In this embodiment a particular value for a is 0.

In this embodiment a particular value for X² is OC(R⁴)₂ wherein each R⁴ is, independently, hydrogen or (1-4C)alkyl. More particularly, X² is OCH₂.

In this embodiment a particular value for Q² is an optionally substituted 5- or 6-membered monocyclic heteroaryl ring, which ring contains 1 nitrogen heteroatom and optionally 1 or 2 (particularly 1) additional heteroatom independently selected from oxygen, nitrogen and sulfur. More particularly Q² is selected from pyridyl, pyrazinyl, 1,3-thiazolyl and 1H-imidazolyl (for example 2-pyridyl, 6-methyl-pyrid-3yl, 3-fluorophenyl, 2-pyrazinyl, 1,3-thiazol-2-yl and 1-methyl-1H-imidazol-2-yl, particularly 2-pyridyl).

In this embodiment a particular value for X³ is a group of the formula —(CR⁸R⁹)ₚ, wherein p is 0, 1 or 2 and each of R⁸ and R⁹, which may be the same or different, is selected from hydrogen and (1-4C)alkyl. For example, a particular value for X³ is —CH₂—.

In this embodiment a particular value for Z is hydrogen, hydroxy, amino, (1-6C)alkylamino or di-[(1-6C)alkyl]amino. More particularly Z is selected from hydrogen and hydroxy.

Another embodiment of the compounds of Formula I is a quinazoline derivative of the Formula IE: 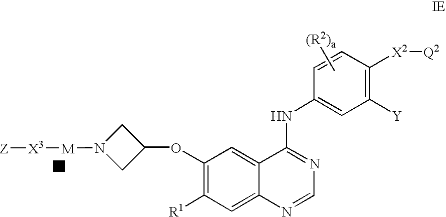
wherein:

R¹ is selected from hydrogen, hydroxy, (1-6C)alkoxy, (3-7C)cycloalkyl-oxy and (3-7C)cycloalkyl-(1-6C)alkoxy,

and wherein adjacent carbon atoms in any (2-6C)alkylene chain within a R¹ substituent are optionally separated by the insertion into the chain of a group selected from O, S, SO, SO₂, N(R³), CO, CON(R³), N(R³)CO, SO₂N(R³) and N(R³)SO₂, wherein R³ is hydrogen or (1-6C)alkyl,

and wherein any CH₂ or CH₃ group within a R¹ substituent optionally bears on each said CH₂ or CH₃ group one or more halogeno or (1-6C)alkyl substituents, or a substituent selected from hydroxy, cyano, amino, carboxy, carbamoyl, sulfamoyl, oxo, thioxo, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino;

Y is selected from hydrogen, halogeno, (1-4C)alkyl, (1-4C)alkoxy, (2-4C)alkenyl and (2-4C)alkynyl;

a is 0, 1, 2 or 3 or 4;

each R², which may be the same or different, is selected from halogeno, (1-4C)alkyl, (1-4C)alkoxy, (2-4C)alkenyl and (2-4C)alkynyl;

X² is a direct bond or is selected from O, S, OC(R⁴)₂, SC(R⁴)₂, SO, SO₂, N(R⁴), CO and N(R⁴)C(R⁴)₂ wherein each R⁴ is, which may be the same or different, is selected from hydrogen or (1-6C)alkyl, and Q² is aryl or heteroaryl,

and wherein Q² optionally bears one or more substituents (for example 1, 2 or 3), which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-1-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, and a group of the formula:
—X⁴—R⁵

wherein X⁴ is a direct bond or is selected from O, CO and N(R⁶), wherein R⁶ is hydrogen or (1-6C)alkyl, and R⁵ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulfamoyl(1-6C)alkyl, N-(1-6C)alkylsulfamoyl(1-6C)alkyl, N,N-di-(1-6C)alkylsulfamoyl(1-6C)alkyl, (2-6C)alkanoyl-(1-6C)alkyl, (2-6C)alkanoyloxy-(1-6C)alkyl or (1-6C)alkoxycarbonyl-(1-6C)alkyl,

and wherein any CH₂ or CH₃ group within —X²-Q² optionally bears on each said CH₂ or CH₃ one or more (for example 1, 2, or 3) halogeno or (1-6C)alkyl substituents or a substituent selected from hydroxy, cyano, amino, (1-4C)alkoxy, (1-4C)alkylamino and di-[(1-4C)alkylamino];

M is selected from CO and SO₂;

X³ is a group of the formula:
—(CR⁸R⁹)ₚ-(Q³)ₘ-(CR¹⁰R¹¹)_{q}—

wherein m is 0 or 1, p is 0, 1, 2, 3 or 4 and q is 0, 1, 2, 3 or 4,

each of R⁸, R⁹, R¹⁰ and R¹¹, which may be the same or different, is selected from hydrogen and (1-6C)alkyl, and

Q³ is selected from (3-7C)cycloalkylene and (3-7C)cycloalkenylene;

Z is selected from hydrogen, hydroxy, amino, (1-6C)alkylamino, di-[(1-6C)alkyl]amino, (1-6C)alkoxy, (1-6C)alkylsulfonyl, (1-6C)alkanesulfonylamino, N-(1-6C)alkyl-(1-6C)alkanesulfonylamino, and a group of the formula:
Q⁴-X⁵—

wherein X⁵ is a direct bond or is selected from O, N(R¹²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,

provided that when X⁵ is a direct bond, Q⁴ is heterocyclyl,

and provided that when m, p and q are all 0, then Z is heterocyclyl,

and wherein adjacent carbon atoms in any (2-6C)alkylene chain within a Z substituent are optionally separated by the insertion into the chain of a group selected from O, S, SO, SO₂, N(R¹³), CO, —C═C— and —C≡C— wherein R¹³ is hydrogen or (1-6C)alkyl,

and wherein any CH₂ or CH₃ group within any Z, X¹ or X³ group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein any heterocyclyl group represented by Q¹ or within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴

wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,

and wherein any heterocyclyl group represented by Q¹ or within a Z substituent optionally bears 1 or 2 oxo or thioxo substituents;

or a pharmaceutically acceptable salt thereof.

In this embodiment a particular value for R¹ is hydrogen.

In this embodiment a particular value for Y is halogeno (such as chloro or fluoro, more particularly chloro) or (1-4C)alkyl (such as methyl).

In this embodiment a particular value for a is 0.

In this embodiment a particular value for X² is O or OC(R⁴)₂ wherein each R⁴ is, independently, hydrogen or (1-4C)alkyl. More particularly, X² is selected from O and OCH₂.

In this embodiment a particular value for Q² is an optionally substituted 5- or 6-membered monocyclic heteroaryl ring, which ring contains 1 nitrogen heteroatom and optionally 1 or 2 (particularly 1) additional heteroatom independently selected from oxygen, nitrogen and sulfur. More particularly Q² is selected from phenyl, pyridyl, pyrazinyl, 1,3-thiazolyl and 1H-imidazolyl (for example 2-pyridyl, 6-methyl-pyrid-3yl, 3-fluorophenyl, 2-pyrazinyl, 1,3-thiazol-2-yl or 1-methyl-1H-imidazol-2-yl, especially 2-pyridyl or 6-methyl-pyrid-3yl).

In this embodiment, a particular value for M is CO.

In this embodiment a particular value for X³ is a group of the formula —(CR⁸R⁹)ₚ, wherein p is 0, 1 or 2 and each of R⁸ and R⁹, which may be the same or different, is selected from hydrogen and (1-4C)alkyl. For example, a particular value for X³ is —CH₂—.

In this embodiment a particular value for Z is hydroxy.

A particular compound of the invention is, for example, one or more quinazoline derivative of the Formula I selected from:
2-{4-[(4-{[3-chloro-4-(pyridin-2-ylmethoxy)phenyl]amino}quinazolin-6-yl)oxy]piperidin-1-yl}-2-oxoethanol;
2-((2S)-2-{[(4-{[3-chloro-4-(pyridin-2-ylmethoxy)phenyl]amino}quinazolin-6-yl)oxy]methyl}pyrrolidin-1-yl)-2-oxoethanol;
2-{(3S)-3-[(4-{[3-chloro-4-(pyridin-2-ylmethoxy)phenyl]amino}quinazolin-6-yl)oxy]piperidin-1-yl}-2-oxoethanol;
6-({1-[(dimethylamino)acetyl]piperidin-4-yl}oxy)-N-{4-[(3-fluorobenzyl)oxy]-3-methoxyphenyl}-7-methoxyquinazolin-4-amine; and
N-[3-chloro-4-(pyrazin-2-ylmethoxy)phenyl]-6-({1-[(dimethylamino)acetyl]piperidin-4-yl}oxy)-7-methoxyquinazolin-4-amine;
   or a pharmaceutically acceptable salt thereof.

Particular compounds of the invention are, for example, one or more quinazoline derivatives of the Formula I selected from:
2-{4-[(4-{[3-chloro-4-(pyridin-2-ylmethoxy)phenyl]amino}quinazolin-6-yl)oxy]piperidin-1-yl}-2-oxoethanol;
2-((2S)-2-{[(4-{[3-chloro-4-(pyridin-2-ylmethoxy)phenyl]amino}quinazolin-6-yl)oxy]methyl}pyrrolidin-1-yl)-2-oxoethanol;
N-[3-chloro-4-(pyridin-2-ylmethoxy)phenyl]-6-({(2S)-1-[(dimethylamino)acetyl]pyrrolidin-2-yl}methoxy)quinazolin-4-amine;
N-[3-chloro-4-(pyridin-2-ylmethoxy)phenyl]-6-({(3S)-1-[(dimethylamino)acetyl]piperidin-3-yl}oxy)quinazolin-4-amine;
2-{(3S)-3-[(4-{[3-chloro-4-(pyridin-2-ylmethoxy)phenyl]amino}quinazolin-6-yl)oxy]pyrrolidin-1-yl}-2-oxoethanol;
2-{(3S)-3-[(4-{[3-chloro-4-(pyridin-2-ylmethoxy)phenyl]amino}quinazolin-6-yl)oxy]piperidin-1-yl}-2-oxoethanol;
N-{3-chloro-4-[(3-fluorobenzyl)oxy]phenyl}-6-({1-[(dimethylamino)acetyl]piperidin-4-yl}oxy)quinazolin-4-amine;
N-[3-chloro-4-(pyridin-2-ylmethoxy)phenyl]-6-({1-[(dimethylamino)acetyl]piperidin-4-yl}oxy)quinazolin-4-amine;
N-[3-chloro-4-(pyrazin-2-ylmethoxy)phenyl]-6-({1-[(dimethylamino)acetyl]piperidin-4-yl}oxy)quinazolin-4-amine;
6-({1-[(dimethylamino)acetyl]piperidin-4-yl}oxy)-N-{4-[(3-fluorobenzyl)oxy]-3-methoxyphenyl}quinazolin-4-amine;
6-({1-[(dimethylamino)acetyl]piperidin-4-yl}oxy)-N-[4-(pyridin-2-ylmethoxy) phenyl]quinazolin-4-amine;
6-({1-[(dimethylamino)acetyl]piperidin-4-yl}oxy)-N-[3-methoxy-4-(pyridin-2-ylmethoxy)phenyl]quinazolin-4-amine;
6-({1-[(dimethylamino)acetyl]piperidin-4-yl}oxy)-N-{4-[(3-fluorobenzyl)oxy) phenyl}-7-methoxyquinazolin-4-amine;
6-({1-[(dimethylamino)acetyl]piperidin-4-yl}oxy)-N-{4-[(3-fluorobenzyl)oxy]-3-methoxyphenyl}-7-methoxyquinazolin-4-amine;
N-{3-chloro-4-[(3-fluorobenzyl)oxy]phenyl}-6-({1-[(dimethylamino)acetyl]piperidin-4-yl}oxy)-7-methoxyquinazolin-4-amine;
6-({1-[(dimethylamino)acetyl]piperidin-4-yl}oxy)-7-methoxy-N-[4-(pyridin-2-ylmethoxy)phenyl]quinazolin-4-amine;
6-({1-[(dimethylamino)acetyl]piperidin-4-yl}oxy)-7-methoxy-N-[3-methoxy-4-(pyridin-2-ylmethoxy)phenyl]quinazolin-4-amine;
N-[3-chloro-4-(pyridin-2-ylmethoxy)phenyl]-6-({1-[(dimethylamino)acetyl]piperidin-4-yl}oxy)-7-methoxyquinazolin-4-amine;
N-[3-chloro-4-(pyrazin-2-ylmethoxy)phenyl]-6-({1-[(dimethylamino)acetyl]piperidin-4-yl}oxy)-7-methoxyquinazolin-4-amine;
6-({1-[(dimethylamino)acetyl]piperidin-4-yl}oxy)-7-methoxy-N-[4-(pyrazin-2-ylmethoxy)phenyl]quinazolin-4-amine;
6-({1-[(dimethylamino)acetyl]piperidin-4-yl}oxy)-7-methoxy-N-[3-methoxy-4-(pyrazin-2-ylmethoxy)phenyl]quinazolin-4-amine;
6-({1-[(dimethylamino)acetyl]piperidin-4-yl}oxy)-N-[4-(3-fluorobenzyloxy)phenyl]quinazolin-4-amine;
6-({1-[(dimethylamino)acetyl]piperidin-4-yl}oxy)-N-[3-methoxy-4-(pyrazin-2-ylmethoxy) phenyl]quinazolin-4-amine;
6-({1-[(dimethylamino)acetyl]piperidin-4-yl}oxy)-N-[4-(pyrazin-2-ylmethoxy) phenyl]quinazolin-4-amine;
N-[3-chloro-4-(pyridin-2-ylmethoxy)phenyl]-6-{[1-(methylsulfonyl)pyrrolidin-3-yl]methoxy}quinazolin-4-amine;
2-{4-[(4-{[3-chloro-4-(pyridin-2-ylmethoxy)phenyl]amino}-7-methoxyquinazolin-6-yl)oxy]piperidin-1-yl}-2-oxoethanol;
6-[(1-acetylpiperidin-4-yl)oxy]-N-[3-chloro-4-(pyridin-2-ylmethoxy)phenyl]-7-methoxyquinazolin-4-amine;
2-{4-[(4-{[3-chloro-(pyrazin-2-ylmethoxy)phenyl]amino}-7-methoxyquinazolin-6-yl)oxy]piperidin-1-yl}-2-oxoethanol;
6-[(1-acetylpiperidin-4-yl)oxy]-N-[3-chloro-(pyrazin-2-ylmethoxy)phenyl]-7-methoxyquinazolin-4-amine;
6-[(1-acetylpiperidin-4-yl)oxy]-N-[3-chloro-4-(pyridin-2-ylmethoxy)phenyl]quinazolin-4-amine;
2-{4-[(4-{[3-chloro-4-(pyrazin-2-ylmethoxy)phenyl]amino}quinazolin-6-yl)oxy]piperidin-1-yl}-2-oxoethanol;
6-[(1-acetylpiperidin-4-yl)oxy]-N-[3-chloro-4-(pyrazin-2-ylmethoxy)phenyl]quinazolin-4-amine;
N-[3-chloro-4-(pyridin-2-ylmethoxy)phenyl]-6-{[1-(methylsulfonyl)piperidin-4-yl]oxy}quinazolin-4-amine;
N-{3-ethynyl-4-[(3-fluorobenzyl)oxy]phenyl}-7-methoxy-6-{[1-(methylsulfonyl)piperidin-4-yl]oxy}quinazolin-4-amine;
7-methoxy-6-{[1-(methylsulfonyl)piperidin-4-yl]oxy}-N-[4-(1,3-thiazol-2-ylthio)phenyl]quinazolin-4-amine;
N-[3-chloro-4-(pyrazin-2-ylmethoxy)phenyl]-6-{[1-(methylsulfonyl)piperidin-4-yl]oxy}quinazolin-4-amine;
N-{3-fluoro-4-[(1-methyl-1H-imidazol-2-yl)thio]phenyl}-6-{[1-(methylsulfonyl)piperidin-4-yl]oxy}quinazolin-4-amine;
N-{3-chloro-4-[(1-methyl-1H-imidazol-2-yl)thio]phenyl}-6-{[1-(methylsulfonyl)piperidin-4-yl]oxy}quinazolin-4-amine;
6-{[1-(methylsulfonyl)piperidin-4-yl]oxy}-N-[4-(1,3-thiazol-2-ylthio)phenyl]quinazolin-4-amine;
N-{3-fluoro-4-[(1-methyl-1H-imidazol-2-yl)thio]phenyl}-7-methoxy-6-{[1-(methylsulfonyl)piperidin-4-yl]oxy}quinazolin-4-amine;
2-(4-{[4-({3-methyl-4-[(6-methylpyridin-3-yl)oxy]phenyl}amino)quinazolin-6-yl]oxy}piperidin-1-yl)-2-oxoethanol;
2-{3-[(4-{[3-chloro-4-(pyridin-2-ylmethoxy)phenyl]amino}quinazolin-6-yl)oxy]azetidin-1-yl}-2-oxoethanol; and
2-(3-{[4-({3-methyl-4-[(6-methylpyridin-3-yl)oxy]phenyl}amino)quinazolin-6-yl]oxy}azetidin-1-yl)-2-oxoethanol;
   or a pharmaceutically acceptable salt thereof.

A particular group of compounds of the invention is, for example, one or more quinazoline derivative of the Formula I selected from:
2-{4-[(4-{[3-chloro-4-(pyridin-2-ylmethoxy)phenyl]amino}quinazolin-6-yl)oxy]piperidin-1-yl)}2-oxoethanol;
2-((2S)-2-{[(4-{[3-chloro-4-(pyridin-2-ylmethoxy)phenyl]amino}quinazolin-6-yl)oxy]methyl}pyrrolidin-1-yl)-2-oxoethanol;
N-[3-chloro-4-(pyridin-2-ylmethoxy)phenyl]-6-({(2S)-1-[(dimethylamino)acetyl]pyrrolidin-2-yl}methoxy)quinazolin-4-amine;
N-[3-chloro-4-(pyridin-2-ylmethoxy)phenyl]-6-({(3S)-1-[(dimethylamino)acetyl]piperidin-3-yl}oxy)quinazolin-4-amine;
2-{(3S)-3-[(4-{[3-chloro-4-(pyridin-2-ylmethoxy)phenyl]amino}quinazolin-6-yl)oxy]pyrrolidin-1-yl}-2-oxoethanol;
2-{(3S)-3-[(4-{[3-chloro-4-(pyridin-2-ylmethoxy)phenyl]amino}quinazolin-6-yl)oxy]piperidin-1-yl}-2-oxoethanol;
N-{3-chloro-4-[(3-fluorobenzyl)oxy]phenyl}-6-({1-[(dimethylamino)acetyl]piperidin-4-yl}oxy)quinazolin-4-amine;
N-[3-chloro-4-(pyridin-2-ylmethoxy)phenyl]-6-({1-[(dimethylamino)acetyl]piperidin-4-yl}oxy)quinazolin-4-amine;
N-[3-chloro-4-(pyrazin-2-ylmethoxy)phenyl]-6-({1-[(dimethylamino)acetyl]piperidin-4-yl}oxy)quinazolin-4-amine;
6-({1-[(dimethylamino)acetyl]piperidin-4-yl}oxy)-N-{4-[(3-fluorobenzyl)oxy]-3-methoxyphenyl}quinazolin-4-amine;
6-({1-[(dimethylamino)acetyl]piperidin-4-yl}oxy)-N-[4-(pyridin-2-ylmethoxy) phenyl]quinazolin-4-amine;
6-({-[(dimethylamino)acetyl]piperidin-4-yl}oxy)-N-[3-methoxy-4-(pyridin-2-ylmethoxy)phenyl]quinazolin-4-amine;
6-({1-[(dimethylamino)acetyl]piperidin-4-yl}oxy)-N-{4-[(3-fluorobenzyl)oxy]phenyl}-7-methoxyquinazolin-4-amine;
6-({1-[(dimethylamino)acetyl]piperidin-4-yl}oxy)-N-{4-[(3-fluorobenzyl)oxy]-3-methoxyphenyl}-7-methoxyquinazolin-4-amine;
N-{3-chloro-4-[(3-fluorobenzyl)oxy]phenyl}-6-({1-[(dimethylamino)acetyl]piperidin-4-yl}oxy)-7-methoxyquinazolin-4-amine;
6-({1-[(dimethylamino)acetyl]piperidin-4-yl}oxy)-7-methoxy-N-[4-(pyridin-2-ylmethoxy)phenyl]quinazolin-4-amine;
6-({1-[(dimethylamino)acetyl]piperidin-4-yl}oxy)-7-methoxy-N-[3-methoxy-4-(pyridin-2-ylmethoxy)phenyl]quinazolin-4-amine;
N-[3-chloro-4-(pyridin-2-ylmethoxy)phenyl]-6-({1-[(dimethylamino)acetyl]piperidin-4-yl}oxy)-7-methoxyquinazolin-4-amine;
N-[3-chloro-4-(pyrazin-2-ylmethoxy)phenyl]-6-({1-[(dimethylamino)acetyl]piperidin-4-yl}oxy)-7-methoxyquinazolin-4-amine;
6-({1-[(dimethylamino)acetyl]piperidin-4-yl}oxy)-7-methoxy-N-[4-(pyrazin-2-ylmethoxy)phenyl]quinazolin-4-amine;
6-({1-[(dimethylamino)acetyl]piperidin-4-yl}oxy)-7-methoxy-N-[3-methoxy-4-(pyrazin-2-ylmethoxy)phenyl]quinazolin-4-amine;
6-({1-[(dimethylamino)acetyl]piperidin-4-yl}oxy)-N-[4-(3-fluorobenzyloxy)phenyl]quinazolin-4-amine;
6-({1-[(dimethylamino)acetyl]piperidin-4-yl}oxy)-N-[3-methoxy-4-(pyrazin-2-ylmethoxy)phenyl]quinazolin-4-amine;
6-({1-[(dimethylamino)acetyl]piperidin-4-yl}oxy)-N-[4-(pyrazin-2-ylmethoxy) phenyl]quinazolin-4-amine;
N-[3-chloro-4-(pyridin-2-ylmethoxy)phenyl]-6-{[1-(methylsulfonyl)pyrrolidin-3-yl]methoxy}quinazolin-4-amine;
   or a pharmaceutically acceptable salt thereof.

Another particular group of compounds of the invention is, for example, one or more quinazoline derivative of the Formula I selected from:
2-{4-[(4-{[3-chloro-4-(pyridin-2-ylmethoxy)phenyl]amino}quinazolin-6-yl)oxy]piperidin-1-yl}-2-oxoethanol;
N-{3-chloro-4-[(3-fluorobenzyl)oxy]phenyl}-6-({1-[(dimethylamino)acetyl]piperidin-4-yl}oxy)-7-methoxyquinazolin-4-amine;
N-[3-chloro-4-(pyridin-2-ylmethoxy)phenyl]-6-({1-[(dimethylamino)acetyl]piperidin-4-yl}oxy)-7-methoxyquinazolin-4-amine;
2-{4-[(4-{[3-chloro-4-(pyridin-2-ylmethoxy)phenyl]amino}-7-methoxyquinazolin-6-yl)oxy]piperidin-1-yl}-2-oxoethanol; and
6-[(1-acetylpiperidin-4-yl)oxy]-N-[3-chloro-4-(pyridin-2-ylmethoxy)phenyl]quinazolin-4-amine;
   or a pharmaceutically acceptable salt thereof.

A particular group of examples of quinazoline derivatives of the Formula IA includes one or more of:
2-{4-[(4-{[3-chloro-4-(pyridin-2-ylmethoxy)phenyl]amino}quinazolin-6-yl)oxy]piperidin-1-yl}-2-oxoethanol;
N-{3-chloro-4-[(3-fluorobenzyl)oxy]phenyl}-6-({1-[(dimethylamino)acetyl]piperidin-4-yl}oxy)quinazolin-4-amine;
N-[3-chloro-4-(pyridin-2-ylmethoxy)phenyl]-6-({1-[(dimethylamino)acetyl]piperidin-4-yl}oxy)quinazolin-4-amine;
N-[3-chloro-4-(pyrazin-2-ylmethoxy)phenyl]-6-({1-[(dimethylamino)acetyl]piperidin-4-yl}oxy)quinazolin-4-amine;
6-({1-[(dimethylamino)acetyl]piperidin-4-yl}oxy)-N-{4-[(3-fluorobenzyl)oxy]-3-methoxyphenyl}quinazolin-4-amine;
6-({1-[(dimethylamino)acetyl]piperidin-4-yl}oxy)-N-[4-(pyridin-2-ylmethoxy) phenyl]quinazolin-4-amine;
6-({1-[(dimethylamino)acetyl]piperidin-4-yl}oxy)-N-[3-methoxy-4-(pyridin-2-ylmethoxy)phenyl]quinazolin-4-amine;
6-({1-[(dimethylamino)acetyl]piperidin-4-yl}oxy)-N-{4-[(3-fluorobenzyl)oxy]phenyl}-7-methoxyquinazolin-4-amine;
6-({1-[(dimethylamino)acetyl]piperidin-4-yl}oxy)-N-{4-[(3-fluorobenzyl)oxy]-3-methoxyphenyl}-7-methoxyquinazolin-4-amine;
N-{3-chloro-4-[(3-fluorobenzyl)oxy]phenyl}-6-({1-[(dimethylamino)acetyl]piperidin-4-yl}oxy)-7-methoxyquinazolin-4-amine;
6-({1-[(dimethylamino)acetyl]piperidin-4-yl}oxy)-7-methoxy-N-[4-(pyridin-2-ylmethoxy)phenyl]quinazolin-4-amine;
6-({1-[(dimethylamino)acetyl]piperidin-4-yl}oxy)-7-methoxy-N-[3-methoxy-4-(pyridin-2-ylmethoxy)phenyl]quinazolin-4-amine;
N-[3-chloro-4-(pyridin-2-ylmethoxy)phenyl]-6-({1-[(dimethylamino)acetyl]piperidin-4-yl}oxy)-7-methoxyquinazolin-4-amine;
N-[3-chloro-4-(pyrazin-2-ylmethoxy)phenyl]-6-({1-[(dimethylamino)acetyl]piperidin-4-yl}oxy)-7-methoxyquinazolin-4-amine;
6-({1-[(dimethylamino)acetyl]piperidin-4-yl}oxy)-7-methoxy-N-[4-(pyrazin-2-ylmethoxy)phenyl]quinazolin-4-amine;
6-({1-[(dimethylamino)acetyl]piperidin-4-yl}oxy)-7-methoxy-N-[3-methoxy-4-(pyrazin-2-ylmethoxy)phenyl]quinazolin-4-amine;
6-({1-[(dimethylamino)acetyl]piperidin-4-yl}oxy)-N-[4-(3-fluorobenzyloxy)phenyl]quinazolin-4-amine;
6-({1-[(dimethylamino)acetyl]piperidin-4-yl}oxy)-N-[3-methoxy-4-(pyrazin-2-ylmethoxy)phenyl]quinazolin-4-amine;
6-({1-[(dimethylamino)acetyl]piperidin-4-yl}oxy)-N-[4-(pyrazin-2-ylmethoxy) phenyl]quinazolin-4-amine;
2-{4-[(4-{[3-chloro-4-(pyridin-2-ylmethoxy)phenyl]amino}-7-methoxyquinazolin-6-yl)oxy]piperidin-1-yl}-2-oxoethanol;
6-[(1-acetylpiperidin-4-yl)oxy]-N-[3-chloro-4-(pyridin-2-ylmethoxy)phenyl]-7-methoxyquinazolin-4-amine;
2-{4-[(4-{[3-chloro-4-(pyrazin-2-ylmethoxy)phenyl]amino}-7-methoxyquinazolin-6-yl)oxy]piperidin-1-yl}-2-oxoethanol;
6-[(1-acetylpiperidin-4-yl)oxy]-N-[3-chloro-4-(pyrazin-2-ylmethoxy)phenyl]-7-methoxyquinazolin-4-amine;
6-[(1-acetylpiperidin-4-yl)oxy]-N-[3-chloro-4-(pyridin-2-ylmethoxy)phenyl]quinazolin-4-amine;
2-{4-[(4-{[3-chloro-4-(pyrazin-2-ylmethoxy)phenyl]amino}quinazolin-6-yl)oxy]piperidin-1-yl}-2-oxoethanol;
6-[(1-acetylpiperidin-4-yl)oxy]-N-[3-chloro-4-(pyrazin-2-ylethoxy)phenyl]quinazolin-4-amine;
N-[3-chloro-4-(pyridin-2-ylmethoxy)phenyl]-6-{[1-(methylsulfonyl)piperidin-4-yl]oxy}quinazolin-4-amine;
N-{3-ethynyl-4-[(3-fluorobenzyl)oxy]phenyl}-7-methoxy-6-{[1-(methylsulfonyl)piperidin-4-yl]oxy}quinazolin-4-amine;
7-methoxy-6-{[1-(methylsulfonyl)piperidin-4-yl]oxy}-N-[4-(1,3-thiazol-2-ylthio)phenyl]quinazolin-4-amine;
N-[3-chloro-4-(pyrazin-2-ylmethoxy)phenyl]-6-{[1-(methylsulfonyl)piperidin-4-yl]oxy}quinazolin-4-amine;
N-{3-fluoro-4-[(1-methyl-1H-imidazol-2-yl)thio]phenyl}-6-{[1-(methylsulfonyl)piperidin-4-yl]oxy}quinazolin-4-amine;
N-{3-chloro-4-[(1-methyl-1H-imidazol-2-yl)thio]phenyl}-6-{[1-(methylsulfonyl)piperidin-4-yl]oxy}quinazolin-4-amine;
6-{[1-(methylsulfonyl)piperidin-4-yl]oxy}-N-[4-(1,3-thiazol-2-ylthio)phenyl]quinazolin-4-amine;
N-{3-fluoro-n-[(1-methyl-1H-imidazol-2-yl)thio]phenyl}-7-methoxy-6-{[1-(methylsulfonyl)piperidin-4-yl]oxy}quinazolin-4-amine; and
2-(4-{[4-({3-methyl-4-[(6-methylpyridin-3-yl)oxy]phenyl}amino)quinazolin-6-yl]oxy}piperidin-1-yl)-2-oxoethanol;
   or a pharmaceutically acceptable salt thereof.

A particular group of examples of quinazoline derivatives of the Formula IB includes one or more of:
N-[3-chloro-4-(pyridin-2-ylmethoxy)phenyl]-6-({(3S)-1-[(dimethylamino)acetyl]piperidin-3-yl}oxy)quinazolin-4-amine; and
2-{(3S)-3-[(4-{[3-chloro-4-(pyridin-2-ylmethoxy)phenyl]amino}quinazolin-6-yl)oxy]piperidin-1-yl}-2-oxoethanol;

or a pharmaceutically acceptable salt thereof.

A particular group of examples of quinazoline derivatives of the Formula IC includes one or more of:
2-((2S)-2-{[(4-{[3-chloro-4-(pyridin-2-ylmethoxy)phenyl]amino}quinazolin-6-yl)oxy]methyl}pyrrolidin-1-yl)-2-oxoethanol; and
N-[3-chloro-4-(pyridin-2-ylmethoxy)phenyl]-6-({(2S)-1-[(dimethylamino)acetyl]pyrrolidin-2-yl}methoxy)quinazolin-4-amine;
   or a pharmaceutically acceptable salt thereof.

A particular group of examples of quinazoline derivatives of the Formula ID includes one or more of:
2-{(3S)-3-[(4-{[3-chloro-4-(pyridin-2-ylmethoxy)phenyl]amino}quinazolin-6-yl)oxy]pyrrolidin-1-yl}-2-oxoethanol; and
N-[3-chloro-4-(pyridin-2-ylmethoxy)phenyl]-6-{[1-(methylsulfonyl)pyrrolidin-3-yl]methoxy}quinazolin-4-amine;
   or a pharmaceutically acceptable salt thereof.

A particular group of examples of quinazoline derivatives of the Formula IE includes one or more of:
2-{3-[(4-{(3-chloro-4-(pyridin-2-ylmethoxy)phenyl]amino}quinazolin-6-yl)oxy]azetidin-1-yl}-2-oxoethanol; and
2-(3-{[4-({3-methyl-4-[(6-methylpyridin-3-yl)oxy]phenyl}amino)quinazolin-6-yl]oxy}azetidin-1-yl)-2-oxoethanol;
   or a pharmaceutically acceptable salt thereof.

A quinazoline derivative of the Formula I, or a pharmaceutically-acceptable salt thereof, may be prepared by any process known to be applicable to the preparation of chemically-related compounds. Suitable processes include, for example, those illustrated in International Patent Applications WO 96/15118, WO01/94341, WO03/040108 and WO03/040109. Such processes, when used to prepare a quinazoline derivative of the Formula I are provided as a further feature of the invention and are illustrated by the following representative process variants in which, unless otherwise stated, R¹, R², X¹, X², X³, Y, M, Q¹, Q², a, and Z have any of the meanings defined hereinbefore. Necessary starting materials may be obtained by standard procedures of organic chemistry. The preparation of such starting materials is described in conjunction with the following representative process variants and within the accompanying Examples. Alternatively necessary starting materials are obtainable by analogous procedures to those illustrated which are within the ordinary skill of an organic chemist.
Process (a) for the preparation of compounds of the Formula I wherein M is CO, the coupling, conveniently in the presence of a suitable base, of a quinazoline of the formula II:

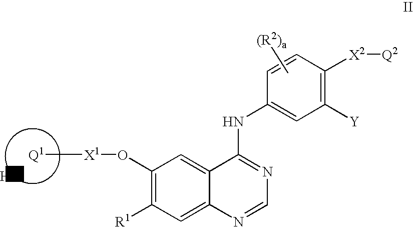

wherein R¹, R², X¹, X² Y, a, Q¹ and Q² have any of the meanings defined hereinbefore except that any functional group is protected if necessary, with a carboxylic acid of the formula III, or a reactive derivative thereof:
Z-X³—COOH III

wherein Z and X³ have any of the meanings defined hereinbefore except that any functional group is protected if necessary;

or

Process (b) the reaction, conveniently in the presence of a suitable base, of a quinazoline of the formula II as hereinbefore defined in relation to Process (a), with a compound of the formula IV:
Z-X³-M-L¹ IV

wherein L¹ is a displaceable group and Z, X³ and M have any of the meanings defined hereinbefore except that any functional group is protected if necessary; or
Process (c) for the preparation of those compounds of the Formula I wherein Z is linked to X³ by nitrogen, the reaction, conveniently in the presence of a suitable base, of a compound of the formula V:

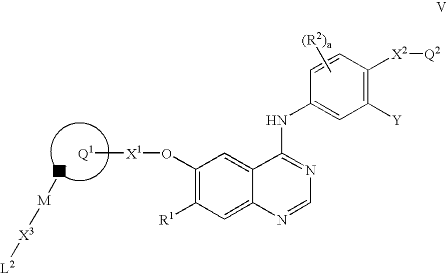

wherein L² is a displaceable group and R¹, R², X¹, X², X³, Y, M, a, Q¹ and Q² have any of the meanings defined hereinbefore except that any functional group is protected if necessary, with a compound of the formula ZH, wherein Z is as hereinbefore defined, except that any functional group is protected if necessary; or
Process (d) the reaction, conveniently in the presence of a suitable base, of a quinazoline of the formula VI:

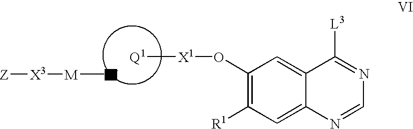

wherein, L³ is a displaceable group and R¹, X¹, X³, Z, and Q¹ have any of the meanings defined hereinbefore except that any functional group is protected if necessary, with a compound of the formula VII:

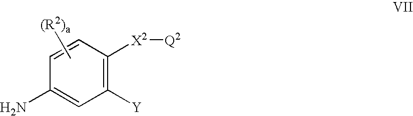

wherein R², a, X², Q² and Y have any of the meanings defined hereinbefore except that any functional group is protected if necessary; or
Process (e) for the preparation of those compounds of the Formula I wherein X² is OC(R⁴)₂, SC(R⁴)₂ or N(R⁴)C(R⁴)₂, the reaction, conveniently in the presence of a suitable base, of a quinazoline of the formula VIII:

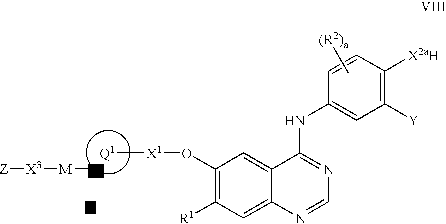

wherein X^{2a} is O, S or N(R⁴) and R¹, R², X¹, X², X³, M, Z, Y, a and Q¹ have any of the meanings defined hereinbefore except that any functional group is protected if necessary, with a compound of the formula IX:
Q²-C(R⁴)₂-L⁴ IX
wherein L⁴ is a suitable displaceable group and Q² and R⁴ have any of the meanings defined hereinbefore except that any functional group is protected if necessary; or
Process (f) for the preparation of those compounds of the Formula I wherein X² is OC(R⁴)₂, the coupling of a quinazoline of the formula X:

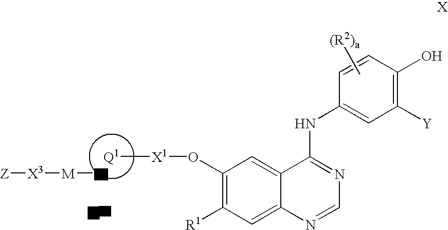

wherein R¹, R², X¹, X², X³, M, Z, Y, a and Q¹ have any of the meanings defined hereinbefore except that any functional group is protected if necessary, with an alcohol of the formula XI:
Q²-C(R⁴)₂—OH XI

wherein Q² and R⁴ have any of the meanings defined hereinbefore except that any functional group is protected if necessary; or
Process (g) the coupling of a quinazoline compound of the formula XII:

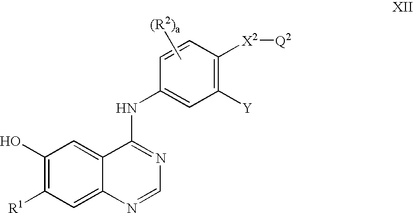

wherein R¹, R², X², a and Y have any of the meanings defined hereinbefore except that any functional group is protected if necessary, with an alcohol of the formula XII:

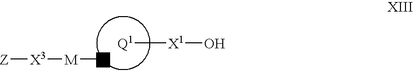

wherein X¹, X³, M, Z, and Q¹ have any of the meanings defined hereinbefore except that any functional group is protected if necessary; or
Process (h) the reaction, conveniently in the presence of a suitable base, of a quinazoline of the formula XII, as hereinbefore defined in relation to Process (g) with a compound of the formula XIV:

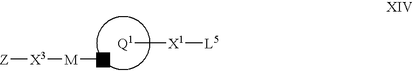

wherein L⁵ is a displaceable group and X¹, X³, M and Z, and Q¹ have any of the meanings defined hereinbefore except that any functional group is protected if necessary;

and thereafter, if necessary:

(i) converting a quinazoline derivative of the formula I into another quinazoline derivative of the formula I;

(ii) removing any protecting group that is present by conventional means;

(iii) forming a pharmaceutically acceptable salt.

Specific conditions for the above reactions are as follows:

Process (a)

The coupling reaction is conveniently carried out in the presence of a suitable coupling agent, such as a carbodiimide, or a suitable peptide coupling agent, for example O-(7-azabenzotriazol-1-yl)-N,N,N′,N′-tetramethyluronium hexafluoro-phosphate (HATU) or a carbodiimide such as dicyclohexylcarbodiimide, optionally in the presence of a catalyst such as dimethylaminopyridine or 4-pyrrolidinopyridine.

The coupling reaction is conveniently carried out in the presence of a suitable base. A suitable base is, for example, an organic amine base such as, for example, pyridine, 2,6-lutidine, collidine, 4-dimethylaminopyridine, triethylamine, di-isopropylethylamine, N-methylmorpholine or diazabicyclo[5.4.0]undec-7-ene, or, for example, an alkali or alkaline earth metal carbonate, for example sodium carbonate, potassium carbonate, cesium carbonate, calcium carbonate.

The reaction is conveniently carried out in the presence of a suitable inert solvent or diluent, for example an ester such as ethyl acetate, a halogenated solvent such as methylene chloride, chloroform or carbon tetrachloride, an ether such as tetrahydrofuran or 1,4-dioxan, an aromatic solvent such as toluene, or a dipolar aprotic solvent such as N,N-dimethylformamide, N,N-dimethylacetamide, N-methylpyrrolidin-2-one or dimethylsulfoxide. The reaction is conveniently carried out at a temperature in the range, for example, from 0 to 120° C., conveniently at or near ambient temperature.

By the term “reactive derivative” of the carboxylic acid of the formula III is meant a carboxylic acid derivative that will react with the quinazoline of formula II to give the corresponding amide. A suitable reactive derivative of a carboxylic acid of the formula III is, for example, an acyl halide, for example an acyl chloride formed by the reaction of the acid and an inorganic acid chloride, for example thionyl chloride; a mixed anhydride, for example an anhydride formed by the reaction of the acid and a chloroformate such as isobutyl chloroformate; an active ester, for example an ester formed by the reaction of the acid and a phenol such as pentafluorophenol, an ester such aspentafluorophenyl trifluoroacetate or an alcohol such as methanol, ethanol, isopropanol, butanol or N-hydroxybenzotriazole; or an acyl azide, for example an azide formed by the reaction of the acid and azide such as diphenylphosphoryl azide; an acyl cyanide, for example a cyanide formed by the reaction of an acid and a cyanide such as diethylphosphoryl cyanide. The reaction of such reactive derivatives of carboxylic acid with amines (such as a compound of the formula II) is well known in the art, for example they may be reacted in the presence of a base (such as those described above), and in a suitable inert solvent (such as those described above). The reaction may conveniently be performed at a temperature as described above.

The quinazoline of the formula II may be obtained by conventional procedures. For example, illustrated in Reaction Scheme 1:

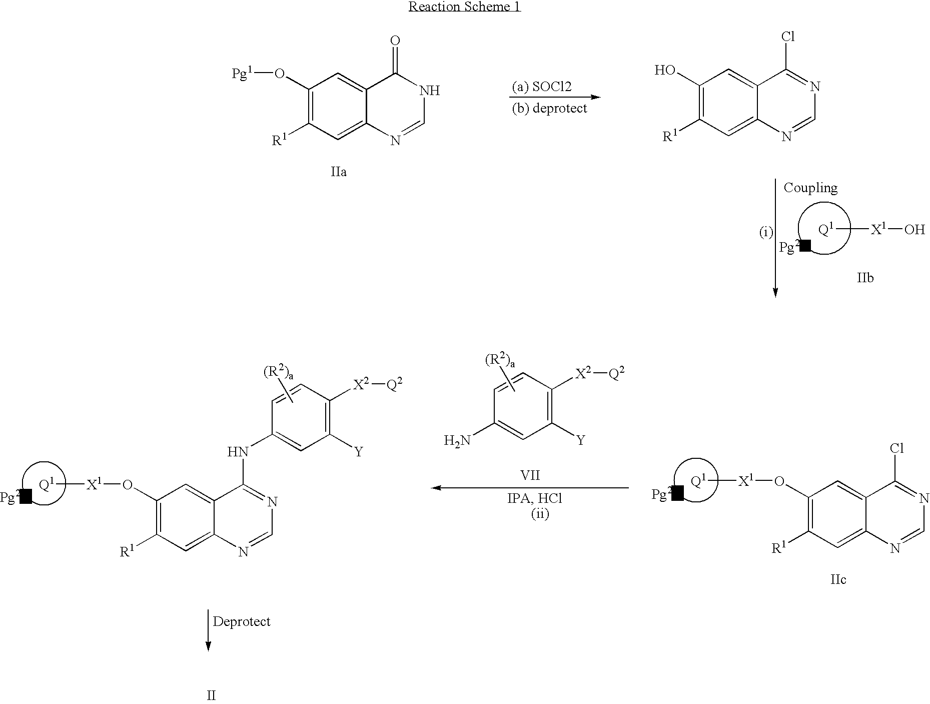

wherein R¹, R², X¹, X², X³, Y, M, Q¹, Q², a, and Z have any of the meanings defined hereinbefore except that any functional group is protected if necessary, and whereafter any protecting group that is present is removed by conventional means; Pg¹ is a suitable hydroxy protecting group (for example a (2-4C)alkanoyl group, such as acetyl); and Pg² is a suitable amino protecting group (for example tert-butoxycarbonyl (BOC)).

Notes:

(i) Coupling under Mitsunobu conditions analogous to those used in Process (f).

(ii) Analogous conditions to Process (d)

The starting quinazoline of formula IIa may be prepared using standard processes known in the art.

Alcohols of the formula IIb are commercially available compounds or they are known in the literature, or they can be can be prepared by standard processes known in the art. For example when X¹ is CH₂ by the reduction of the corresponding acid or ester thereof as illustrated in Reaction Scheme 2: 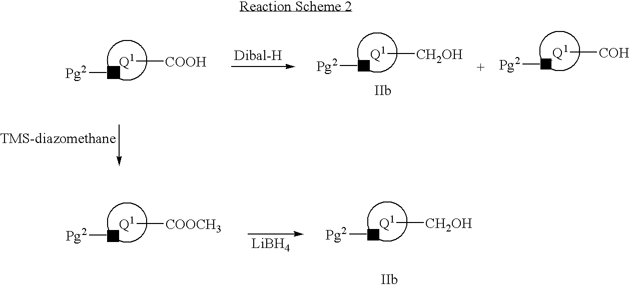
Process (b)

A suitable displaceable group L¹ includes for example halogeno such as chloro.

The reaction is conveniently performed in the presence of a suitable base, for example, conveniently in the presence of a suitable base, for example an organic amine base such as, for example, pyridine, 2,6-lutidine, collidine, 4-dimethylaminopyridine, triethylamine, di-isopropylethylamine, N-methylmorpholine or diazabicyclo[5.4.0]undec-7-ene, or, for example, an alkali or alkaline earth metal carbonate, for example sodium carbonate, potassium carbonate, cesium carbonate, calcium carbonate, or, for example, an alkali metal hydride, for example sodium hydride.

The reaction is conveniently carried out in the presence of a suitable inert solvent or diluent, for example a halogenated solvent such as methylene chloride, chloroform or carbon tetrachloride, an ether such as tetrahydrofuran or 1,4-dioxane, an aromatic solvent such as toluene, or a dipolar aprotic solvent such as N,N-dimethylformamide, N,N-dimethylacetamide, N-methylpyrrolidin-2-one or dimethylsulfoxide.

The compound of the formula IV are commercially available compounds or they are known in the literature, or they can be can be prepared by standard processes known in the art.

Process (c):

A suitable displaceable group represented by L² includes, for example a halogeno or a sulfonyloxy group, for example chloro, bromo, methylsulfonyloxy or toluene-4-sulfonyloxy group. A particular group L¹ is chloro.

The reaction is conveniently performed in the presence of a suitable base, for example one of the bases described in relation to Process (a).

The reaction is conveniently carried out in the presence of a suitable inert solvent or diluent, for example a halogenated solvent such as methylene chloride, chloroform or carbon tetrachloride, an ether such as tetrahydrofuran or 1,4-dioxane, an ester such as ethyl acetate, an aromatic solvent such as toluene, or a dipolar aprotic solvent such as N,N-dimethylformamide, N,N-dimethylacetamide, N-methylpyrrolidin-2-one or dimethylsulfoxide.

The compound of formula V used as starting material may be prepared by, for example, reacting, conveniently in the presence of a suitable base, a compound of the formula II, as hereinbefore defined in relation to Process (a), with a compound of the formula Va:
L²-X³-M-L⁴ Va

wherein X³ and M are as hereinbefore defined, and L² and L⁴ are suitable displaceable groups, provided that L is more labile than L².

Suitable displaceable groups represented by L² and L¹ include for example halogeno such as chloro.

The reaction is conveniently carried out in the presence of a suitable base and in a suitable inert solvent or diluent as defined above for the reaction of the quinazoline of formula V with the compound of the formula ZH.

The compounds of the formulae ZH and Va are commercially available compounds or they are known in the literature, or they can be can be prepared by standard processes known in the art.

Conveniently, in an embodiment of Process (c), a quinazoline of Formula I may be prepared directly from a quinazoline of formula II by reacting the quinazoline of formula II with a compound of formula Va and then reacting the resultant product directly with the compound of the formula ZH without isolating the compound of formula V. This reaction enables the quinazoline of Formula I to be prepared in a single reaction vessel starting with the quinazoline of formula II.

Process (d)

A suitable displaceable group represented by L³ includes, for example, a halogeno (particularly chloro), alkoxy, aryloxy, mercapto, alkylthio, arylthio, alkylsulfinyl, arylsulfinyl, alkylsulfonyl, arylsulfonyl, alkylsulfonyloxy or arylsulfonyloxy group, for example a chloro, bromo, methoxy, phenoxy, pentafluorophenoxy, methylthio, methanesulfonyl, methanesulfonyloxy or toluene-4-sulfonyloxy group.

The reaction is conveniently carried out in the presence of an acid. Suitable acids include, for example hydrogen chloride gas (conveniently dissolved in diethyl ether or dioxane) or hydrochloric acid.

Alternatively the quinazoline derivative of the formula VI, wherein L³ is halogeno (for example chloro), may be reacted with the compound of the formula VII in the absence of an acid or a base. In this reaction displacement of the halogeno leaving group L³ results in the formation of the acid HL³ in-situ and the autocatalysis of the reaction.

Alternatively, the reaction of the quinazoline of formula VI with the compound of formula VII may be carried out in the presence of a suitable base. A suitable base is, for example, a base as defined in relation to Process (a) such as an organic amine base for example, di-isopropylethylamine.

The above reactions are conveniently carried out in the presence of a suitable inert solvent or diluent, for example an alcohol or ester such as methanol, ethanol, isopropanol or ethyl acetate, a halogenated solvent such as methylene chloride, chloroform or carbon tetrachloride, an ether such as tetrahydrofuran or 1,4-dioxan, an aromatic solvent such as toluene, or a dipolar aprotic solvent such as N,N-dimethylformamide, N,N-dimethylacetamide, N-methylpyrrolidin-2-one or dimethylsulfoxide. The above reactions are conveniently carried out at a temperature in the range, for example, 0 to 250° C., conveniently in the range 40 to 80° C. or, preferably, at or near the reflux temperature of the solvent when used.

The quinazoline of the formula VI may be prepared using conventional procedures, for example by coupling the quinazoline of the formula VIa: 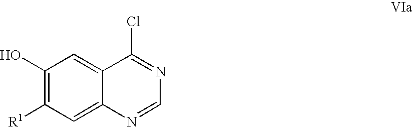
wherein R¹ is as hereinbefore defined, except that any functional group is protected if necessary, with an alcohol of the formula XIII as hereinbefore defined, and whereafter any protecting group that is present is removed by conventional means.

The coupling reaction is suitably carried out under Mitsunobu conditions analogous to those described hereinafter in relation to Process (f). The quinazoline of formula VIa my be prepared as described in Reaction Scheme 1.

Compounds of the formula VII are commercially available compounds or they are known in the literature, or they can be prepared by standard processes known in the art. For example, the compound of the formula VII wherein X² is O, S, N(R⁴), OC(R⁴)₂, SC(R⁴)₂ or N(R⁴)C(R⁴)₂ may be prepared in accordance with Reaction Scheme 3:

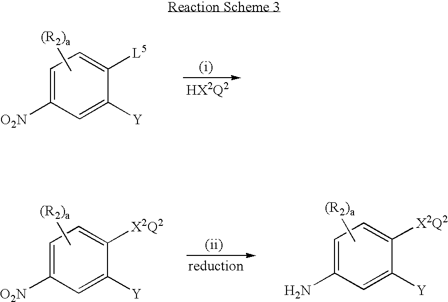

wherein L⁵ is a suitable displaceable group as hereinbefore defined (for example halogeno such as chloro) and Q², X², Y, R¹ and a are as hereinbefore defined, except any functional group is protected if necessary, and whereafter any protecting group that is present is removed by conventional means.

(i) The compounds of the formula HX²Q² are commercially available, or they are known in the literature, or can be prepared using well known processes in the art. For example compounds of the formula Q²CH₂OH may be prepared using known methods, for example by reduction of the corresponding ester of the formula Q²COOR, wherein R is, for example (1-6C)alkyl, or benzyl, with a suitable reducing agent, for example sodium borohydride, followed by ester hydrolysis.

(ii) The reduction of the nitro group in step (ii) may be carried out under standard conditions, for example by catalytic hydrogenation over a platinum/carbon, palladium/carbon, platinum oxide or nickel catalyst, treatment with a metal such as iron, titanium chloride, tin II chloride or indium, or treatment with another suitable reducing agent such as sodium dithionite.

Compounds of the formula VII wherein X² is OC(R⁴)₂, SC(R⁴)₂ or N(R⁴)C(R⁴)₂ may, for example, be prepared in accordance with Reaction Scheme 4: 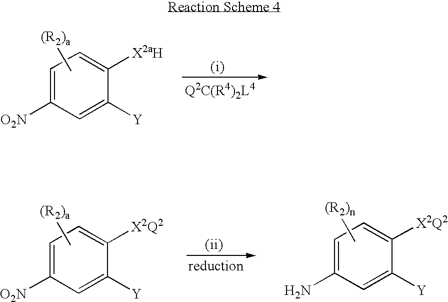
wherein L¹ is a suitable leaving group as defined hereinafter in relation to Process (e), and X^{2a} is as hereinbefore defined in Process (e).
Step (i): Analogous conditions to those used in Process (e)
Step (ii) Analogous conditions to those used in Reaction Scheme 3.

Compounds of the formula VII wherein X² is OC(R⁴)₂ may also be prepared by coupling the appropriate starting nitro phenol in Reaction Scheme 4 (X^{2a}H is OH) with a compound of the formula Q²C(R⁴)₂OH, conveniently in under Mitsunobu conditions analogous to these described hereinafter for Process (f).

Process (e)

A suitable displaceable group L⁴ in the compound of the formula IX is for example halogeno or a sulfonyloxy group, for example fluoro, chloro, methylsulfonyloxy or toluene-4-sulfonyloxy group. A particular group L⁴ is fluoro or chloro or methylsulfonyloxy.

The reaction of the quinazoline of formula VIII with the compound of formula IX is conveniently carried out in the presence of a suitable base such as, for example, a base as described in relation to Process (a) such as an alkali or alkaline earth metal carbonate, for example potassium carbonate.

The reaction a quinazoline of the formula VIII and the compound of the formula IX is conveniently carried out in the presence of a suitable inert solvent or diluent, for example a halogenated solvent such as methylene chloride, chloroform or carbon tetrachloride, an ether such as tetrahydrofuran or 1,4-dioxane, an aromatic solvent such as toluene, or a dipolar aprotic solvent such as N,N-dimethylformamide, N,N-dimethylacetamide, N-methylpyrrolidin-2-one or dimethylsulfoxide. The reaction is conveniently carried out at a temperature in the range of; for example, from 25 to 100° C., conveniently at or near ambient temperature.

Compounds of the formula IX are commercially available compounds or they are known in the literature, or they can be can be prepared by standard processes known in the art.

The quinazoline of the formula VIII may be prepared using conventional methods, for example, by reacting a compound of the formula VI (as defined in relation to process (d)) with a compound of the formula VIIIa:

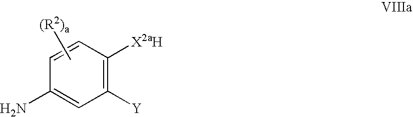

wherein R², X²⁸, a and Y are as hereinbefore defined, except that any functional group is protected if necessary, and whereafter any protecting group that is present is removed by conventional means. The reaction is suitably carried out using analogous conditions to those used in Process (d).

Compounds of the formula VIIIa are commercially available compounds or they are known in the literature, or they can be can be prepared by standard processes known in the art.

Process (f)

The coupling of the quinazoline of formula X with the alcohol of the formula XI is conveniently carried out using the Mitsunobu coupling reaction. Suitable Mitsunobu conditions are well known and include, for example, reaction in the presence of a suitable tertiary phosphine and a di-alkylazodicarboxylate in an organic solvent such as THF, or suitably dichloromethane and in the temperature range 0° C. to 60° C., but suitably at or near ambient temperature. A suitable tertiary phosphine includes for example tri-n-butylphosphine or particularly tri-phenylphosphine. A suitable di-alkylazodicarboxylate includes, for example, diethyl azodicarboxylate (DEAD) or suitably di-tert-butyl azodicarboxylate (DTAD) or di-isopropyl azodicarboxylate. Details of Mitsunobu reactions are contained in Tet. Letts., 31, 699, (1990); The Mitsunobu Reaction, D. L. Hughes, Organic Reactions, 1992, Vol. 42, 335-656 and Progress in the Mitsunobu Reaction, D. L. Hughes, Organic Preparations and Procedures International, 1996, Vol. 28, 127-164.

The quinazoline of the formula X can be prepared by, for example, a compound of the formula VI (as defined in relation to process (d)) with a compound of the formula VIIIa, wherein the group X^{2a}H is OH. Compounds of the formula XI are commercially available compounds or they are known in the literature, or they can be can be prepared by standard processes known in the art.

Process (g) The coupling reaction may be carried out using analogous conditions to those described above for Process (f).

The quinazoline of formula XII may be prepared using conventional techniques, for example by reacting a quinazoline of the formula VIa as hereinbefore defined with an aniline of the formula VII as hereinbefore defined. The reaction may be carried out using analogous conditions to those described above for Process (d).

The alcohol of formula XIII used as a starting material may be prepared using conventional methods. The alcohol of the formula XIII may be prepared using conventional procedures well known in the art, such as those illustrated by the examples herein.

Process (h) A suitable leaving group represented by L⁵ includes for example halogeno such as chloro or bromo. The reaction may be carried out in the presence of a suitable base such as one of those described herein in relation to Process (b). The reaction may be carried out using analogous conditions to those described above for Process (b).

The compound of formula XIV may be prepared using conventional techniques.

The quinazoline derivative of the Formula I may be obtained from the above processes in the form of the free base or alternatively it may be obtained in the form of a salt, an acid addition salt. When it is desired to obtain the free base from a salt of the compound of Formula I, the salt may be treated with a suitable base, for example, an alkali or alkaline earth metal carbonate or hydroxide, for example sodium carbonate, potassium carbonate, calcium carbonate; sodium hydroxide or potassium hydroxide, or by treatment with ammonia for example using a methanolic ammonia solution such as 7N ammonia in methanol.

The protecting groups used in the processes above may in general be chosen from any of the groups described in the literature or known to the skilled chemist as appropriate for the protection of the group in question and may be introduced by conventional methods. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower”, as in, for example, lower alkyl, signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned are, of course, within the scope of the invention.

A carboxy protecting group may be the residue of an ester-forming aliphatic or arylaliphatic alcohol or of an ester-forming silanol (the said alcohol or silanol preferably containing 1-20 carbon atoms). Examples of carboxy protecting groups include straight or branched chain (1-12C)alkyl groups (for example isopropyl, and tert-butyl); lower alkoxy-lower alkyl groups (for example methoxymethyl, ethoxymethyl and isobutoxymethyl); lower acyloxy-lower alkyl groups, (for example acetoxymethyl, propionyloxymethyl, butyryloxymethyl and pivaloyloxymethyl); lower alkoxycarbonyloxy-lower alkyl groups (for example 1-methoxycarbonyloxyethyl and 1-ethoxycarbonyloxyethyl); aryl-lower alkyl groups (for example benzyl, 4-methoxybenzyl, 2-nitrobenzyl, 4-nitrobenzyl, benzhydryl and phthalidyl); tri(lower alkyl)silyl groups (for example trimethylsilyl and tert-butyldimethylsilyl); tri(lower alkyl)silyl-lower alkyl groups (for example trimethylsilylethyl); and (2-6C)alkenyl groups (for example allyl). Methods particularly appropriate for the removal of carboxyl protecting groups include for example acid-, base-, metal- or enzymically-catalysed cleavage.

Examples of hydroxy protecting groups include lower alkyl groups (for example tert-butyl), lower alkenyl groups (for example allyl); lower alkanoyl groups (for example acetyl); lower alkoxycarbonyl groups (for example tert-butoxycarbonyl); lower alkenyloxycarbonyl groups (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); tri(lower alkyl)silyl (for example trimethylsilyl and tert-butyldimethylsilyl) and aryl-lower alkyl (for example benzyl) groups.

Examples of amino protecting groups include formyl, aryl-lower alkyl groups (for example benzyl and substituted benzyl, 4-methoxybenzyl, 2-nitrobenzyl and 2,4-dimethoxybenzyl, and triphenylmethyl); di-4-anisylmethyl and furylmethyl groups; lower alkoxycarbonyl (for example tert-butoxycarbonyl); lower alkenyloxycarbonyl (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); lower alkanoyloxyalkyl groups (for example pivaloyloxymethyl); trialkylsilyl (for example trimethylsilyl and tert-butyldimethylsilyl); alkylidene (for example methylidene) and benzylidene and substituted benzylidene groups.

Methods appropriate for removal of hydroxy and amino protecting groups include, for example, acid-, base-, metal- or enzymically-catalysed hydrolysis for groups such as 2-nitrobenzyloxycarbonyl, hydrogenation for groups such as benzyl and photolytically for groups such as 2-nitrobenzyloxycarbonyl. For example a tert butoxycarbonyl protecting group may be removed from an amino group by an acid catalysed hydrolysis using trifluoroacetic acid.

The reader is referred to Advanced Organic Chemistry, 4th Edition, by J. March, published by John Wiley & Sons 1992, for general guidance on reaction conditions and reagents and to Protective Groups in Organic Synthesis, 2^{nd} Edition, by T. Green et al., also published by John Wiley & Son, for general guidance on protecting groups.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group.

When a pharmaceutically-acceptable salt of a quinazoline derivative of the formula I is required, for example an acid-addition salt, it may be obtained by, for example, reaction of said quinazoline derivative with a suitable acid using a conventional procedure.

As mentioned hereinbefore some of the compounds according to the present invention may contain one of more chiral centers and may therefore exist as stereoisomers (for example when Q¹ is pyrrolidin-2-yl). Stereoisomers may be separated using conventional techniques, e.g. chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions which will not cause racemisation or epimerisation, or by derivatisation, with a chiral reagent. When a specific stereoisomer is isolated it is suitably isolated substantially free for other stereoisomers, for example containing less than 20%, particularly less than 10% and more particularly less than 5% by weight of other stereoisomers.

In the section above relating to the preparation of the quinazoline derivative of Formula I, the expression “inert solvent” refers to a solvent which does not react with the starting materials, reagents, intermediates or products in a manner which adversely affects the yield of the desired product.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

Certain intermediates used in the processes described above are novel and form a further feature of the present invention. Accordingly there is provided a compound of the formula II, or a salt thereof. The intermediate may be in the form of a salt of the intermediate. Such salts need not be a pharmaceutically acceptable salt. For example it may be useful to prepare an intermediate in the form of a pharmaceutically non-acceptable salt if, for example, such salts are useful in the manufacture of a compound of Formula I.

Biological Assays

The inhibitory activities of compounds were assessed in non-cell based protein tyrosine kinase assays as well as in cell based proliferation assays before their in vivo activity was assessed in Xenograft studies.

A) Protein Tyrosine Kinase Phosphorylation Assays

This test measures the ability of a test compound to inhibit the phosphorylation of a tyrosine containing polypeptide substrate by EGFR tyrosine kinase enzyme.

Recombinant intracellular fragments of EGFR, erbB2 and erbB4 (accession numbers X00588, X03363 and L07868 respectively) were cloned and expressed in the baculovirus/Sf21 system. Lysates were prepared from these cells by treatment with ice-cold lysis buffer (20 mM N-2-hydroxyethylpiperizine-N′-2-ethanesulfonic acid (HEPES) pH7.5, 150 mM NaCl, 10% glycerol, 1% Triton X-100, 1.5 mM MgCl₂, 1 mM ethylene glycol-bis(β-aminoethyl ether) N′,N′,N′,N′-tetraacetic acid (EGTA), plus protease inhibitors and then cleared by centrifugation.

Constitutive kinase activity of these recombinant proteins was determined by their ability to phosphorylate a synthetic peptide (made up of a random co-polymer of Glutamic Acid, Alanine and Tyrosine in the ratio of 6:3:1). Specifically, Maxisorb™ 96-well immunoplates were coated with synthetic peptide (0.2 μg of peptide in a 200 μl phosphate buffered saline (PBS) solution and incubated at 4° C. overnight). Plates were washed in 50 mM HEPES pH 7.4 at room temperature to remove any excess unbound synthetic peptide. EGFR or erbB2 activities were assessed by incubation in peptide coated plates for 20 minutes at room temperature in 100 mM HEPES pH 7.4 at room temperature, adenosine trisphosphate (ATP) at Km concentration for the respective enzyme, 10 mM MnCl₂, 0.1M Na₃VO₄, 0.2 mM DL-dithiothreitol (DTT), 0.1% Triton X-100 with test compound in DMSO (final concentration of 2.5%). Reactions were terminated by the removal of the liquid components of the assay followed by washing of the plates with PBS-T (phosphate buffered saline with 0.5% Tween 20).

The immobilised phospho-peptide product of the reaction was detected by immunological methods. Firstly, plates were incubated for 90 minutes at room temperature with anti-phosphotyrosine primary antibodies that were raised in the mouse (4G10 from Upstate Biotechnology). Following extensive washing, plates were treated with Horseradish Peroxidase (HRP) conjugated sheep anti-mouse secondary antibody (NXA931 from Amersham) for 60 minutes at room temperature. After further washing, HRP activity in each well of the plate was measured calorimetrically using 22′-Azino-di-[3-ethylbenzthiazoline sulfonate (6)] diammonium salt crystals (ABTS™ from Roche) as a substrate.

Quantification of colour development and thus enzyme activity was achieved by the measurement of absorbance at 405 nm on a Molecular Devices ThermoMax microplate reader. Kinase inhibition for a given compound was expressed as an IC₅₀ value. This was determined by calculation of the concentration of compound that was required to give 50% inhibition of phosphorylation in this assay. The range of phosphorylation was calculated from the positive (vehicle plus ATP) and negative (vehicle minus ATP) control values.

B) EGFR Driven KB Cell Proliferation Assay

This assay measures the ability of a test compound to inhibit the proliferation of KB cells (human naso-pharangeal carcinoma obtained from the American Type Culture Collection (ATCC)).

KB cells were cultured in Dulbecco's modified Eagle's medium (DMEM) containing 10% foetal calf serum, 2 mM glutamine and non-essential amino acids at 37° C. in a 7.5% CO₂ air incubator. Cells were harvested from the stock flasks using Trypsin/ethylaminediaminetetraacetic acid (EDTA). Cell density was measured using a haemocytometer and viability was calculated using trypan blue solution before being seeded at a density of 1.25×10³ cells per well of a 96 well plate in DMEM containing 2.5% charcoal stripped serum, 1 mM glutamine and non-essential amino acids at 37° C. in 7.5% CO₂ and allowed to settle for 4 hours.

Following adhesion to the plate, the cells are treated with or without EGF (final concentration of 1 ng/ml) and with or without compound at a range of concentrations in dimethylsulfoxide (DMSO) (0.1% final) before incubation for 4 days. Following the incubation period, cell numbers were determined by addition of 50 μl of 3-(4,5-Dimethylthiazol-2-yl)-2,5-diphenyltetrazolium bromide (MTT) (stock 5 mg/ml) for 2 hours. MTT solution was then tipped off, the plate gently tapped dry and the cells dissolved upon the addition of 100 μl of DMSO.

Absorbance of the solubilised cells was read at 540 nm using a Molecular Devices ThermoMax microplate reader. Inhibition of proliferation was expressed as an IC₅₀ value. This was determined by calculation of the concentration of compound that was required to give 50% inhibition of proliferation. The range of proliferation was calculated from the positive (vehicle plus EGF) and negative (vehicle minus EGF) control values.

c) Clone 24 phospho-erbB2 Cell Assay

This immunofluorescence end point assay measures the ability of a test compound to inhibit the phosphorylation of erbB2 in a MCF7 (breast carcinoma) derived cell line which was generated by transfecting MCF7 cells with the full length erbB2 gene using standard methods to give a cell line that overexpresses full length wild type erbB2 protein (hereinafter ‘Clone 24’ cells).

Clone 24 cells were cultured in Growth Medium (phenol red free Dulbecco's modified Eagle's medium (DMEM) containing 10% foetal bovine serum, 2 mM glutamine and 1.2 mg/ml G418) in a 7.5% CO₂ air incubator at 37° C. Cells were harvested from T75 stock flasks by washing once in PBS phosphate buffered saline, pH7.4, Gibco No. 10010-015) and harvested using 2 mls of Trypsin (1.25 mg/ml)/ethylaminediaminetetraacetic acid (EDTA) (0.8 mg/ml) solution. The cells were resuspended in Growth Medium. Cell density was measured using a haemocytometer and viability was calculated using Trypan Blue solution before being further diluted in Growth Medium and seeded at a density of 1×10⁴ cells per well (in 100 ul) into clear bottomed 96 well plates (Packard, No. 6005182).

3 days later, Growth Medium was removed from the wells and replaced with 100 ul Assay Medium (phenol red free DMEM, 2 mM glutamine, 1.2 mg/ml G418) either with or without erbB inhibitor compound. Plates were returned to the incubator for 4 hrs and then 20 μl of 20% formaldehyde solution in PBS was added to each well and the plate was left at room temperature for 30 minutes. This fixative solution was removed with a multichannel pipette, 100 μl of PBS was added to each well and then removed with a multichannel pipette and then 50 μl PBS was added to each well. Plates were then sealed and stored for up to 2 weeks at 4° C.

Immunostaining was performed at room temperature. Wells were washed once with 200 μl PBS/Tween 20 (made by adding 1 sachet of PBS/Tween dry powder (Sigma, No. P3563) to 1 L of double distilled H₂O) using a plate washer then 200 μl Blocking Solution (5% Marvel dried skimmed milk (Nestle) in PBS/Tween 20) was added and incubated for 10 minutes. Blocking Solution was removed using a plate washer and 200 μl of 0.5% Triton X-100/PBS was added to permeabalise the cells. After 10 minutes, the plate was washed with 200 μl PBS/Tween 20 and then 200 μl Blocking Solution was added once again and incubated for 15 minutes. Following removal of the Blocking Solution with a plate washer, 30 μl of rabbit polyclonal anti-phospho ErbB2 IgG antibody (epitope phospho-Tyr 1248, SantaCruz, No. SC-12352-R), diluted 1:250 in Blocking Solution, was added to each well and incubated for 2 hours. Then this primary antibody solution was removed from the wells using a plate washer followed by two 200 μl PBS/Tween 20 washes using a plate washer. Then 30 μl of Alexa-Fluor 488 goat anti-rabbit IgG secondary antibody (Molecular Probes, No. A-11008), diluted 1:750 in Blocking Solution, was added to each well. From now onwards, wherever possible, plates were protected from light exposure, at this stage by sealing with black backing tape. The plates were incubated for 45 minutes and then the secondary antibody solution was removed from the wells followed by two 200 ul PBS/Tween 20 washes using a plate washer. Then 100 μl PBS was added to each plate, incubated for 10 minutes and then removed using a plate washer. Then a further 100 PBS was added to each plate and then, without prolonged incubation, removed using a plate washer. Then 50 μl of PBS was added to each well and plates were resealed with black backing tape and stored for up to 2 days at 4° C. before analysis.

The Fluorescence signal is each well was measured using an Acumen Explorer Instrument (Acumen Bioscience Ltd.), a plate reader that can be used to rapidly quantitate features of images generated by laser-scanning. The instrument was set to measure the number of fluorescent objects above a pre-set threshold value and this provided a measure of the phosphorylation status of erbB2 protein. Fluorescence dose response data obtained with each compound was exported into a suitable software package (such as Origin) to perform curve fitting analysis. Inhibition of erbB2 phosphorylation was expressed as an IC₅₀ value. This was determined by calculation of the concentration of compound that was required to give 50% inhibition of erbB2 phosphorylation signal.

d) In Vivo BT-474 Xenograft Assay

This assay measures the ability of a test compound to inhibit the growth of a BT-474 tumour cell xenograft (human mammary carcinoma obtained from Dr Baselga, Laboratorio Recerca Oncologica, Paseo Vall D'Hebron 119-129, Barcelona 08035, Spain) in Female Swiss athymic mice (Alderley Park, nu/nu genotype) (Baselga, J. et al. (1998) Cancer Research, 58, 2825-2831).

Female Swiss athymic (nu/nu genotype) mice were bred and maintained in Alderley Park in negative pressure Isolators (PFI Systems Ltd.). Mice were housed in a barrier facility with 12 hr light/dark cycles and provided with sterilised food and water ad libitum. All procedures were performed on mice of at least 8 weeks of age. BT-474 tumour cell xenografts were established in the hind flank of donor mice by sub-cutaneous injections of 1×10⁷ freshly cultured cells in 100 μl of serum free media with 50% Matrigel per animal. On day 14 post-implant, mice were randomised into groups of 10 prior to the treatment with compound or vehicle control that was administered once daily at 0.1 ml/10 g body weight. Tumour volume was assessed twice weekly by bilateral Vernier calliper measurement, using the formula (length×width)×√(length×width)×(π/6), where length was the longest diameter across the tumour, and width was the corresponding perpendicular. Growth inhibition from start of treatment was calculated by comparison of the mean changes in tumour volume for the control and treated groups, and statistical significance between the two groups was evaluated using a Students t test.

Although the pharmacological properties of the compounds of the Formula I vary with structural change as expected, in general activity possessed by compounds of the Formula I, may be demonstrated at the following concentrations or doses in one or more of the above tests (a), (b) and (c):—

Test (a):—IC₅₀ in the range, for example, 0.001-1 μM;

Test (b):—IC₅₀ in the range, for example, 0.001-5 μM;

Test (c):—IC₅₀ in the range, for example, 0.001-5 μM;

Test (d):—activity in the range, for example, 1-200 mg/kg/day;

No physiologically unacceptable toxicity was observed in Test (d) at the effective dose for compounds tested of the present invention. Accordingly no untoward toxicological effects are expected when a compound of Formula I, or a pharmaceutically-acceptable salt thereof, as defined hereinbefore is administered at the dosage ranges defined hereinafter.

By way of example, Table A illustrates the activity of representative compounds according to the invention. Column 2 of Table A shows IC₅₀ data from Test (a) for the inhibition of EGFR tyrosine kinase protein phosphorylation; column 3 shows IC₅₀ data from Test (a) for the inhibition of erbB2 tyrosine kinase protein phosphorylation; and column 4 shows IC₅₀ data for inhibition of phosphorylation of erbB2 in a MCF7 derived cell line in Test (c) described above:

TABLE A

| | IC₅₀ (μM) | IC₅₀ (μM) | IC₅₀ (μM) |
|---|---|---|---|
| | Test (a): | Test (a): | Test (c): |
| | Inhibition of | Inhibition of | Inhibition of |
| | EGFR tyrosine | erbB2 tyrosine | erbB2 tyrosine |
| Example | kinase protein | kinase protein | kinase protein |
| Number | phosphorylation | phosphorylation | phosphorylation |
| 8 | 0.039 | 0.002 | 0.210 |
| 9 | 0.021 | 0.007 | 0.150 |
| 14 | 0.213 | 0.002 | 0.004 |

According to a further aspect of the invention there is provided a pharmaceutical composition which comprises a quinazoline derivative of the formula I, or a pharmaceutically-acceptable thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a quinazoline derivative of the formula I will naturally vary according to the nature and severity of the conditions, the age and sex of the animal or patient and the route of administration, according to well known principles of medicine.

In using a quinazoline derivative of the formula I for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used Oral administration is however preferred, particularly in tablet form Typically, unit dosage forms will contain about 0.5 mg to 0.5 g of a compound of this invention.

We have found that the compounds of the present invention possess anti-proliferative properties such as anti-cancer properties that are believed to arise from their erb-B, particularly EGFR and more particularly erbB2 receptor tyrosine kinase inhibitory activity. Furthermore, certain of the compounds according to the present invention possess substantially better potency against the erbB2 receptor tyrosine kinase, than against other tyrosine kinases enzymes, such as EGFR tyrosine kinase. Such compounds possess sufficient potency against the erbB2 receptor tyrosine kinase that they may be used in an amount sufficient to inhibit erbB2 receptor tyrosine kinase whilst demonstrating little, or significantly lower, activity against other tyrosine kinases such as EGFR. Such compounds are likely to be useful for the selective inhibition of erbB2 receptor tyrosine kinase and are likely to be useful for the effective treatment of, for example erbB2 driven tumours. Accordingly, the compounds of the present invention are expected to be useful in the treatment of diseases or medical conditions mediated alone or in part by and erb-B, particularly erbB2 receptor tyrosine kinases, i.e. the compounds may be used to produce a erb-B, particularly an erbB2, receptor tyrosine kinase inhibitory effect in a warm-blooded animal in need of such treatment. Thus the compounds of the present invention provide a method for the treatment of malignant cells characterised by inhibition of the erb-B, particularly erbB2, receptor tyrosine kinase. Particularly the compounds of the invention may be used to produce an anti-proliferative and/or pro-apoptotic and/or anti-invasive effect mediated alone or in part by the inhibition of erb-B, particularly erbB2, receptor tyrosine kinases. Particularly, the compounds of the present invention are expected to be useful in the prevention or treatment of those tumours that are sensitive to inhibition of an erb-B, particularly the erbB2, receptor tyrosine kinase that are involved in the signal transduction steps which drive proliferation and survival of these tumour cells. Accordingly the compounds of the present invention are expected to be useful in the treatment and/or prevention of a number of hyperproliferative disorders by providing an anti-proliferative effect. These disorders include, for example psoriasis, benign prostatic hyperplasia (BPH), atherosclerosis and restenosis and, in particular, erb-B, more particularly erb-B2, receptor tyrosine kinase driven tumours. Such benign or malignant tumours may affect any tissue and include non-solid tumours such as leukaemia, multiple myeloma or lymphoma, and also solid tumours, for example bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval tumours.

According to this aspect of the invention there is provided a quinazoline derivative of the formula I, or a pharmaceutically acceptable salt thereof, for use as a medicament.

Thus according to this aspect of the invention there is provided the use of a quinazoline derivative of the formula I, or a pharmaceutically-acceptable salt thereof, as defined hereinbefore in the manufacture of a medicament for use in the production of an anti-proliferative effect in a warm-blooded animal such as man.

According to a further feature of this aspect of the invention there is provided a method for producing an anti-proliferative effect in a warm-blooded animal, such as man, in need of such treatment which comprises administering to said animal an effective amount of a quinazoline derivative of the formula I, or a pharmaceutically acceptable salt thereof, as hereinbefore defined.

According to a further aspect of the invention there is provided a quinazoline derivative of the formula I, or a pharmaceutically acceptable salt thereof, for use in the production of an anti-proliferative effect in a warm-blooded animal such as man.

According to a further aspect of the invention there is provided the use of a quinazoline derivative of the formula I, or a pharmaceutically-acceptable salt thereof, as defined hereinbefore in the manufacture of a medicament for use in the production of an anti-proliferative effect which effect is produced alone or in part by inhibiting erbB2 receptor tyrosine kinase in a warm-blooded animal such as man.

According to a further feature of this aspect of the invention there is provided a method for producing an anti-proliferative effect which effect is produced alone or in part by inhibiting erbB2 receptor tyrosine kinase in a warm-blooded animal, such as man, in need of such treatment which comprises administering to said animal an effective amount of a quinazoline derivative of the formula I, or a pharmaceutically acceptable salt thereof, as hereinbefore defined.

According to a further aspect of the invention there is provided a quinazoline derivative of the formula I, or a pharmaceutically acceptable salt thereof, for use in the production of an anti-proliferative effect which effect is produced alone or in part by inhibiting erbB2 receptor tyrosine kinase in a warm-blooded animal such as man.

According to a further aspect of the present invention there is provided the use of a quinazoline derivative of the formula I, or a pharmaceutically-acceptable salt thereof, as defined hereinbefore in the manufacture of a medicament for use in the treatment of a disease or medical condition (for example a cancer as mentioned herein) mediated alone or in part by erb-B, particularly erbB2, receptor tyrosine kinase.

According to a further feature of this aspect of the invention there is provided a method for treating a disease or medical condition (for example a cancer as mentioned herein) mediated alone or in part by erb-B, particularly erbB2, receptor tyrosine kinase in a warm-blooded animal, such as man, in need of such treatment, which comprises administering to said animal an effective amount of a quinazoline derivative of the formula I, or a pharmaceutically-acceptable salt thereof, as defined hereinbefore.

According to a further aspect of the invention there is provided a quinazoline derivative of the formula I, or a pharmaceutically acceptable salt thereof, for use in the treatment of a disease or medical condition (for example a cancer as mentioned herein) mediated alone or in part by erb-B, particularly erbB2, receptor tyrosine kinase.

According to a further aspect of the invention there is provided the use of a quinazoline derivative of the formula I, or a pharmaceutically-acceptable salt thereof, as defined hereinbefore in the manufacture of a medicament for use in the prevention or treatment of those tumours which are sensitive to inhibition of erbB2 receptor tyrosine kinase that is involved in the signal transduction steps which lead to the proliferation of tumour cells.

According to a further feature of this aspect of the invention there is provided a method for the prevention or treatment of those tumours which are sensitive to inhibition of erbB2 receptor tyrosine kinase, that is involved in the signal transduction steps which lead to the proliferation and/or survival of tumour cells in a warm-blooded animal, such as man, in need of such treatment, which comprises administering to said animal an effective amount of a quinazoline derivative of the formula I, or a pharmaceutically-acceptable salt thereof, as defined hereinbefore.

According to a further aspect of the invention there is provided a quinazoline derivative of the formula I, or a pharmaceutically acceptable salt thereof, for use in the prevention or treatment of those tumours which are sensitive to inhibition of the erbB2 receptor tyrosine kinase, that is involved in the signal transduction steps which lead to the proliferation and/or survival of tumour cells. According to a further aspect of the invention there is provided the use of a quinazoline derivative of the formula I, or a pharmaceutically-acceptable salt thereof, as defined hereinbefore in the manufacture of a medicament for use in providing a erbB2 receptor tyrosine kinase inhibitory effect.

According to a further feature of this aspect of the invention there is provided a method for providing an erbB2 receptor tyrosine kinase inhibitory effect in a warm-blooded animal, such as man, in need of such treatment, which comprises administering to said animal an effective amount of a quinazoline derivative of the formula I, or a pharmaceutically-acceptable salt thereof, as defined hereinbefore.

According to a further aspect of the invention there is provided a quinazoline derivative of the formula I, or a pharmaceutically acceptable salt thereof, for use in providing an erbB2 receptor tyrosine kinase inhibitory effect.

According to a further aspect of the invention there is provided the use of a quinazoline derivative of the formula I, or a pharmaceutically-acceptable salt thereof, as defined hereinbefore in the manufacture of a medicament for use in providing a selective erbB2 kinase inhibitory effect.

According to a further feature of this aspect of the invention there is provided a method for providing a selective erbB2 kinase inhibitory effect in a warm-blooded animal, such as man, in need of such treatment, which comprises administering to said animal an effective amount of a quinazoline derivative of the formula I, or a pharmaceutically-acceptable salt thereof, as defined hereinbefore.

According to a further aspect of the invention there is provided a quinazoline derivative of the formula I, or a pharmaceutically acceptable salt thereof, for use in providing a selective erbB2 kinase inhibitory effect.

By “a selective erbB2 kinase inhibitory effect” is meant that the quinazoline derivative of Formula I is more potent against erbB2 receptor tyrosine kinase than it is against other kinases. In particular some of the compounds according to the invention are more potent against erbB2 receptor kinase than it is against other tyrosine kinases such as other erb-B receptor tyrosine kinases, particularly EGFR tyrosine kinase. For example a selective erb-B2 kinase inhibitor according to the invention is at least 5 times, preferably at least 10 times more potent against erbB2 receptor tyrosine kinase than it is against EGFR tyrosine kinase, as determined from the relative IC₅₀ values in suitable assays (for example the by comparing the IC₅₀ value from the Clone 24 phospho-erbB2 cell assay (a measure of the erb-B2 tyrosine kinase inhibitory activity in cells) with the IC₅₀ from the KB cell assay (a measure of the EGFR tyrosine kinase inhibitory activity in cells) for a given test compound as described above).

According to a further aspect of the present invention there is provided the use of a quinazoline derivative of the formula I, or a pharmaceutically-acceptable salt thereof, as defined hereinbefore in the manufacture of a medicament for use in the treatment of a cancer, for example a cancer selected from leukaemia, multiple myeloma, lymphoma, bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval cancer.

According to a further feature of this aspect of the invention there is provided a method for treating a cancer, for example a cancer selected from selected from leukaemia, multiple myeloma, lymphoma, bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval cancer in a warm-blooded animal, such as man, in need of such treatment, which comprises administering to said animal an effective amount of a quinazoline derivative of the formula I, or a pharmaceutically-acceptable salt thereof, as defined hereinbefore.

According to a further aspect of the invention there is provided a quinazoline derivative of the formula I, or a pharmaceutically acceptable salt thereof, for use in the treatment of a cancer, for example a cancer selected from leukaemia, multiple myeloma, lymphoma, bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, uterine and vulval cancer.

The anti-proliferative treatment defined hereinbefore may be applied as a sole therapy or may involve, in addition to the quinazoline derivative of the invention, conventional surgery or radiotherapy or chemotherapy. Such chemotherapy may include one or more of the following categories of anti-tumour agents:

As mentioned above the size of the dose required for the therapeutic or prophlyactic treatment of a particular disease will necessarily be varied depending upon, amongst other things, the host treated, the route of administration and the severity of the illness being treated.

The anti-proliferative treatment defined hereinbefore may be applied as a sole therapy or may involve, in addition to the quinazoline derivative of the invention, conventional surgery or radiotherapy or chemotherapy. Such chemotherapy may include one or more of the following categories of anti-tumour agents:—

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulfan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbB2 antibody trastuzumab [Herceptin™] and the anti-erbB1 antibody cetuximab [C225]), farnesyl transferase inhibitors, tyrosine kinase inhibitors and serine/threonine kinase inhibitors, for example other inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;

(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);

(vi) vascular damaging agents such as Combretastatin A4 and compounds disclosed in International Patent Applications WO 99/02166, WO00/40529, WO 00/41669, WO01/92224, WO02/04434 and WO02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a pharmaceutical product comprising a quinazoline derivative of the Formula I as defined hereinbefore and an additional anti-tumour agent as defined hereinbefore for the conjoint treatment of cancer.

Although the compounds of the Formula I are primarily of value as therapeutic agents for use in warm-blooded animals (including man), they are also useful whenever it is required to inhibit the effects of the erbB receptor tyrosine protein kinases. Thus, they are useful as pharmacological standards for use in the development of new biological tests and in the search for new pharmacological agents.

The invention will now be illustrated by the following non limiting examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous magnesium sulfate; evaporation of solvent was carried out using a rotary evaporator under reduced pressure (600-4000 Pascals; 4.5-30 mmHg) with a bath temperature of up to 60° C.;

(iii) chromatography means flash chromatography on silica gel; thin layer chromatography (TLC) was carried out on silica gel plates;

(iv) in general, the course of reactions was followed by TLC and/or analytical LC-MS, and reaction times are given for illustration only;

(v) final products had satisfactory proton nuclear magnetic resonance (NMR) spectra and/or mass spectral data;

(vi) yields are given for illustration only and are not necessarily those which can be obtained by diligent process development; preparations were repeated if more material was required;

(vii) when given, NMR data is in the form of delta values for major diagnostic protons, given in parts per million (ppm) relative to tetramethylsilane (TMS) as an internal standard, determined at 300 MHz using perdeuterio dimethyl sulfoxide (DMSO-d₆) as solvent unless otherwise indicated; the following abbreviations have been used: s, singlet; d, doublet; t, triplet; q, quartet; m, multiplet; b, broad;

(viii) chemical symbols have their usual meanings; SI units and symbols are used;

(ix) solvent ratios are given in volume:volume (v/v) terms; and

(x) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺ which refers to the protonated mass ion; reference to M⁺ is to the mass ion generated by loss of an electron; and reference to M-H⁺ is to the mass ion generated by loss of a proton;

(xi) unless stated otherwise compounds containing an asymmetrically substituted carbon and/or sulfur atom have not been resolved;

(xii) where a synthesis is described as being analogous to that described in a previous example the amounts used are the millimolar ratio equivalents to those used in the previous example;

(xiii) all microwave reactions were carried out in a CEM Discover™ microwave synthesisor;

(xiv) preparative high performance liquid chromatography (HPLC) was performed on a Gilson instrument using the following conditions:

Column: 21 mm×10 cm Hichrom RPB

Solvent A: Water+0.1% trifluoroacetic acid,

Solvent B: Acetonitrile+0.1% trifluoroacetic acid

Flow rate: 18 ml/min

Run time: 15 minutes with a 10 minute gradient from 5-95% B

Wavelength: 254 nm, bandwidth 10 nm

Injection volume 2.0-4.0 ml;

(xv) the following abbreviations have been used:

HATU O-(7-Azabenzotriazol-1-yl)-N,N,N′,N′-Tetramethyluronium Hexafluoro-Phosphate;
DIAD diisopropyl azodicarboxylate;
THF tetrahydrofuran;
DMF N,N-dimethylformamide;
DMA N,N-dimethylacetamide;
DCM dichloromethane;
DMSO dimethylsulfoxide;
IPA isopropyl alcohol;
ether diethyl ether;
TFA trifluoroacetic acid;
EtOAc ethyl acetate;

### EXAMPLE 1

### 2-{4-[(4-{[3-Chloro-4-(pyridin-2-ylmethoxy)phenyl]amino)quinazolin-6-yl)oxy]piperidin-1-yl}-2-oxoethanol

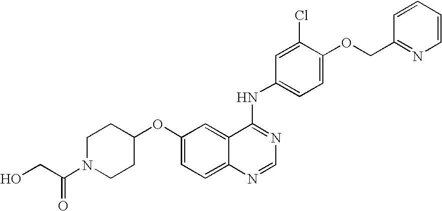

A mixture of HATU (234 mg), N,N-diisopropylethylamine (715 μl), glycolic acid (47 mg) and N-[3-chloro-4-(pyridin-2-ylmethoxy)phenyl]-6-(piperidin-4-yloxy)quinazolin-4-amine (189 mg) in DCM (5 ml) was stirred overnight. The solution was concentrated in vacuo and the residue purified by chromatography using EtOAc to DCM-5% methanol as eluant. The resultant product was treated with a polymer-supported carbonate (ex. Argonaut technologies) to give the title compound as a white solid (65 mg, 31%); NMR spectrum (DMSO-d6) 1.60-1.80 (m, 2H), 1.95-2.11 (m, 2H), 3.32-3.49 (m, 2H), 3.58-3.69 (m, 1H), 4.13 (d, 2H), 4.53 (t, 1H), 4.80-4.90 (m, 1H), 5.31 (s, 2H), 7.30 (d, 1H), 7.35-7.40 (m, 1H), 7.58 (dd, 1H), 7.60 (d, 1H), 7.72 (dd, 1H), 7.75 (d, 1H), 7.89 (dt, 1H), 7.95 (d, 1H), 8.00 (d, 1H), 8.49 (s, 1H), 8.60 (dt, 1H) and 9.54 (s, 1H); Mass spectrum MH⁺ 520.

The N-[3-chloro-4-(pyridin-2-ylmethoxy)phenyl]-6-(piperidinyloxy)quinazolin-4-amine used as starting material was prepared as follows:

DMF (500 μl) was added to a suspension of 6-acetoxy-3,4-dihydro-3H-quinazolin-4-one (6.0 g) in thionyl chloride (45 ml) and the mixture was stirred and heated at 90° C. for 3 hours. Volatile material was removed by evaporation and the residue was azeotroped with toluene (20 ml) to give 4-chloroquinazolin-6-yl acetate (7.61 g, 99%) as a solid which was used without purification; NMR spectrum (CDCl₃) 9.10 (s, 1H), 8.19 (s, 1H), 8.03 (d, 1H), 7.95 (dd, 1H), 2.38 (s, 3H).

4-Chloroquinazolin-6-yl acetate (7.61 g) was dissolved in 7N ammonia in methanol (100 ml) and stirred under nitrogen for 1 h. The solution was reduced in volume to about 2 ml and triturated with diethyl ether to give 4-chloroquinazolin-6-ol (4.20 g, 80%) as a beige solid; NMR spectrum (DMSO-d6) 8.85 (s, 1H), 7.96 (d, 1H), 7.61 (dd, 1H), 7.40 (d, 1H).

4-Chloroquinazolin-6-ol (250 mg) in DCM (10 ml) was treated with triphenylphosphine (540 mg), 1-tert-butoxycarbonyl-4-hydroxypiperidine (414 mg) and DIAD (420 mg) and stirred under nitrogen for 20 hours. The solution was purified by chromatography using ethyl acetate-isohexane as eluant to give tert-butyl 4-[(4-chloroquinazolin-6-yl)oxy]piperidine-1-carboxylate (96%) as a white solid; Mass spectrum MH⁺ 364.

tert-butyl 4-[(4-chloroquinazolin-6-yl)oxy]piperidine-1-carboxylate (580 mg) in IPA (8 ml) containing N,N-diisopropylethylamine (28 μl) was treated with 3-chloro-4-(pyridin-2-ylmethoxy)aniline (377 mg, obtained as described in Example 13 of WO 96/15118) and heated at 80° C. for 4 hours. The mixture was cooled, treated with HCl (4M in dioxane) (1.61 ml) and stirred overnight. The solution was concentrated in vacuo and the residue purified by chromatography using DCM-5% methanol-0.2% NH₄OH as elegant to give N-[3-chloro-4-(pyridin-2-ylmethoxy)phenyl]-6-(piperidin-4-yloxy)quinazolin-4-amine (191 mg, 25%); Mass spectrum MH⁺ 462.

### EXAMPLE 2

### 2-((2S)-2-{[(4-{[3-Chloro-4-(pyridin-2-ylmethoxy)phenyl]amino}quinazolin-6-yl)oxy]methyl}pyrrolidin-1-yl)-2-oxoethanol

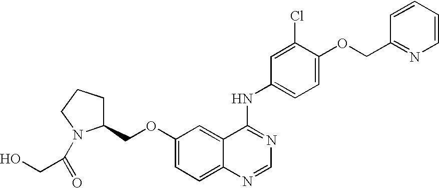

The procedure described in example 1 was repeated using glycolic acid and N-[3-chloro-4-(pyridin-2-ylmethoxy)phenyl]-6-[(2S)-pyrrolidin-2-ylmethoxy]quinazolin-4-amine in 39% yield; NMR spectrum (DMSO-d6) 1.88-2.15 (m, 4H), 3.35-3.50 (m, 2H), 4.40-4.15 (m, 3H), 4.23-4.30 (m, 1H), 4.37-4.44 (m, 1H), 4.62 (t, 1H), 5.30 (s, 1H), 7.28 (d, 1H), 7.38 (ddd, 1H), 7.52 (d, 1H), 7.60 (d, 1H), 7.73 (d, 1H), 7.79 (dd, 1H), 7.89 (dt, 1H), 7.98 (d, 1H), 8.70 (d, 1H), 8.50 (s, 1H), 8.60 (dt, 1H); Mass spectrum MH⁺ 520.

The N-[3-chloro-4-(pyridin-2-ylmethoxy)phenyl]-6-[(2S)-pyrrolidin-2-ylmethoxy]quinazolin-4-amine used as starting material was prepared as follows:

The procedure described in example 1 (preparation of starting materials) was repeated using 4-chloroquinazolin-6-ol and tert-butyl (2S)-2-(hydroxymethyl)pyrrolidine-1-carboxylate to give tert-butyl (2S)-2-{[(4-chloroquinazolin-6-yl)oxy]methyl}pyrrolidine-1-carboxylate as a white solid in 90% yield; Mass spectrum MH⁺ 364.

Tert-butyl (2S)-2-{[(4-chloroquinazolin-6-yl)oxy]methyl}pyrrolidine-1-carboxylate was then reacted with 3-chloro-4-(pyridin-2-ylmethoxy)aniline using the same procedure described in example 1 (preparation of starting materials) to give N-[3-chloro-4-(pyridin-2-ylmethoxy)phenyl]-6-[(2S)-pyrrolidin-2-ylmethoxy]quinazolin-4-amine in 20% yield; Mass spectrum MH⁺ 462.

### EXAMPLE 3

### N-[3-Chloro-4-(pyridin-2-ylmethoxy)phenyl]-6-({(2S)-1-[(dimethylamino)acetyl]pyrrolidin-2-yl}methoxy)quinazolin-4-amine

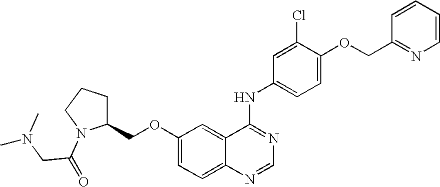

The procedure described in example 1 was repeated using N,N-dimethylglycine and N-[3-chloro-4-(pyridin-2-ylmethoxy)phenyl]-6-[(2S)-pyrrolidin-2-ylmethoxy]quinazolin-4-amine in 22% yield, NMR spectrum (DMSO-d6) 1.88-2.17 (m, 4H), 2.22 (s, 6H), 3.09 (dd, 2H), 3.47-3.65 (m, 2H), 4.13 (dd, 1H), 4.24 (dd, 1H), 4.34-4.42 (m, 1H), 5.30 (s, 2H), 7.27 (d, 1H), 7.38 (dd, 1H), 7.51 (dd, 1H), 7.60 (d, 1H), 7.72 (d, 1H), 7.82 (dd, 1H), 7.89 (dt, 1H), 8.04 (d, 1H), 8.09 (d, 1H), 8.51 (s, 1H), 8.61 (d, 1H), 9.53 (s, 1H); Mass spectrum MH⁺ 547.

The N-[3-chloro-4-(pyridin-2-ylmethoxy)phenyl]-6-[(2S)-pyrrolidin-2-ylmethoxy]quinazolin-4-amine used as starting material was prepared as described in example 2 (preparation of starting materials).

### EXAMPLE 4

### N-[3-Chloro-4-(pyridin-2-ylmethoxy)phenyl]-6-({(3S)-1-[(dimethylamino)acetyl]piperidin-3-yl}oxy)quinazolin-4-amine

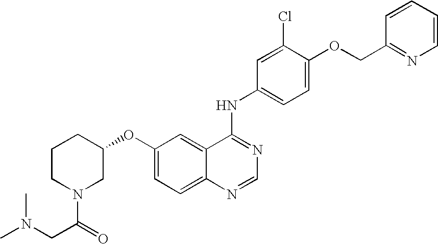

The procedure described in example 1 was repeated using N,N-dimethylglycine and N-[3-chloro-4-(pyridin-2-ylmethoxy)phenyl]-6-[(3S)-piperidin-3-yloxy]quinazolin-4-amine in 4% yield; NMR spectrum (DMSO-d6) 1.52-1.63 (m, 1H), 1.80-1.97 (m, 1H), 1.65-1.79 (m, 1H), 2.03-2.17 (m, 1H), 2.81 (s, 3H), 2.83 (s, 3H), 3.42-3.52 (m, 1H), 3.53-3.59 (m, 1H), 3.67-3.82 (m, 1H), 4.15 (dt, 1H), 4.37 (ddd, 1H), 4.71 (dd, 1H), 5.09 (dt, 1H), 5.40 (s, 2H), 7.37 (d, 1H), 7.49 (dd, 1H), 7.68-7.81 (d+m, 3H), 7.94-8.05 (m, 3H), 8.67 (d, 1H), 8.85-8.90 (m, 1H), 9.02-9.05 (m, 1H), 9.57-9.69 (m, 1H) and 12.20 (s, 1H), 12.36; Mass spectrum MH⁺ 547.

The N-[3-chloro-4-(pyridin-2-ylmethoxy)phenyl]-6-[(3S)-piperidin-3-yloxy]quinazolin-4-amine used as starting material was prepared as follows:

The procedure described in example 1 (preparation of starting materials) was repeated using 4-chloroquinazolin-6-ol and tert-butyl (3R)-3-hydroxypiperidine-1-carboxylate to give tert-butyl (3S)-3-[(4-chloroquinazolin-6-yl)oxy]piperidine-1-carboxylate as a white solid in 3% yield; Mass spectrum M⁺ 364.

(3S)-3-[(4-chloroquinazolin-6-yl)oxy]piperidine-1-carboxylate was then reacted with 3-chloro-4-(pyridin-2-ylmethoxy)aniline using the procedure described in example 1 (preparation of starting materials) to give N-[3-chloro-4-(pyridin-2-ylmethoxy)phenyl]-6-[(3S)-piperidin-3-yloxy]quinazolin-4-amine in 42% yield; Mass spectrum MH⁺ 462.

### EXAMPLE 5

### 2-{(3S)-3-[(4-{[3-Chloro-4-(pyridin-2-ylmethoxy)phenyl]amino}quinazolin-6-yl)oxy]pyrrolidin-1-yl}-2-oxoethanol

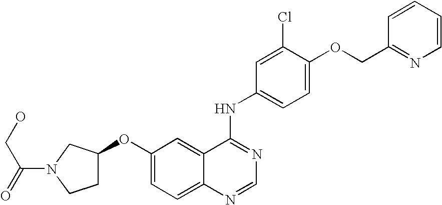

The procedure described in example 1 was repeated using glycolic acid and N-[3-chloro-4-(pyridin-2-ylmethoxy)phenyl]-6-[(3S)-pyrrolidin-3-yloxy]quinazolin-4-amine in 14% yield; NMR spectrum (DMSO-d6) 2.11-2.35 (m, 2H), 3.42-3.57 (m, 1H), 3.59-3.84 (m+dd, 3H), 4.01 (t, 1H), 4.07 (d, 1H), 4.60 (dt, 1H), 5.27 (d, 1H), 5.31 (s, 2H), 7.29 (s, 1H), 7.38 (ddd, 1H), 7.51-7.57 (m, 1H), 7.60 (d, 1H), 7.69-7.78 (m, 2H), 7.88 (dd, 1H), 7.92 (dd, 1H), 7.98-8.02 (m, 1H), 8.50 (d, 1H), 8.59-8.62 (m, 1H), 9.58 (m, 1H); Mass spectrum MH⁺ 506.

The N-[3-chloro-4-(pyridin-2-ylmethoxy)phenyl]-6-[(3S)-pyrrolidin-3-yloxy]quinazolin-4-amine used as starting material was prepared as follows:

The procedure described in example 1 (preparation of starting materials) was repeated using 4-chloroquinazolin-6-ol and tert-butyl (3R)-3-hydroxypyrrolidine-1-carboxylate to give tert-butyl (3S)-3-[(4-chloroquinazolin-6-yl)oxy]pyrrolidine-1-carboxylate as a white solid in 90% yield; Mass spectrum MH⁺ 350.

tert-butyl (3S)-3-[(4-chloroquinazolin-6-yl)oxy]pyrrolidine-1-carboxylate was reacted with 3-chloro-4-(pyridin-2-ylmethoxy)aniline using the same procedure described in example 1 (preparation of starting materials) to give N-[3-chloro-4-(pyridin-2-ylmethoxy)phenyl]-6-[(3S)-pyrrolidin-3-yloxy]quinazolin-4-amine in 40% yield; Mass spectrum MH⁺ 462.

### EXAMPLE 6

### 2-{(3S)-3-[(4-{[3-Chloro-4-(pyridin-2-ylmethoxy)phenyl]amino}quinazolin-6-yl)oxy]piperidin-1-yl}-2-oxoethanol

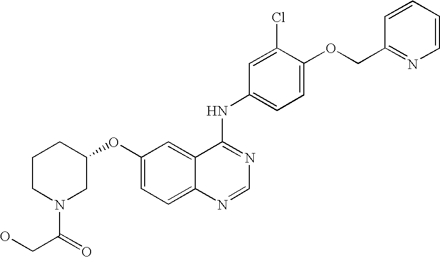

The procedure described in example 1 was repeated using glycolic acid and N-[3-chloro-4-(pyridin-2-ylmethoxy)phenyl]-6-[(3S)-piperidin-3-yloxy]quinazolin-4-amine in 21% yield; NMR spectrum (DMSO-d6) 1.49-1.65 (m, 1H), 1.70-1.94 (m, 2H), 2.00-2.15 (m, 1H), 3.35-3.58 (m, 2H), 3.59-4.20 (m, 3H), 3.80-3.95 (m, 1H), 4.50-4.78 (m, 2H), 5.34 (m, 2H), 7.32 (m, 1H), 7.35-7.40 (m, 1H), 7.50-7.55 (m, 1H), 7.56-7.63 (m, 1H), 7.68-7.80 (m, 2H), 8.85-8.05 (m, 3H), 8.52 (s, 1H), 8.62 (d, 1H), 9.58 (s, 1H); Mass spectrum MH⁺ 520.

The N-[3-chloro-4-(pyridin-2-ylmethoxy)phenyl]-6-[(3S)-piperidin-3-yloxy]quinazolin-4-amine used as starting material was prepared as described in example 4 (preparation of starting materials).

### EXAMPLE 7

### N-(3-chloro-4-[(3-fluorobenzyl)oxy]phenyl}-6-({1-[(dimethylamino)acetyl]piperidin-4-yl}oxy)quinazolin-4-amine

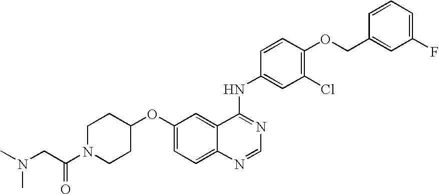

Chloroacetyl chloride (42 μl, 0.52 mmol) was added to an ice-cooled mixture of N-{3-chloro-4-[(3-fluorobenzyl)oxy]phenyl}-6-[(piperidin-4-yl)oxy]quinazolin-4-amine (250 mg, 0.52 mmol) and N,N-diisopropylethylamine (0.11 ml, 0.63 mmol) in dichloromethane (4 ml). The mixture was stirred at room temperature for 1 hour then 3M dimethylamine in dioxane (0.52 ml, 1.56 mmol) was added. The mixture was stirred for 2 hours at room temperature, then diluted in dichloromethane. The organic layer was washed with water and dried over magnesium sulfate. After evaporation of the solvents under vacuum, the residue was purified by chromatography on silica gel (eluant: 3% to 5% 7N methanolic ammonia in dichloromethane) to give the title compound as a white solid (170 mg, 58%); NMR Spectrum: (CDCl₃) 1.8-2.0 (m, 4H), 2.26 (s, 6H), 3.13 (m, 2H), 3.5-3.7 (m, 2H), 3.8-3.9 (m, 2H), 4.69 (m, 1H), 5.16 (s, 2H), 6.97 (d, 1H), 7.02 (m, 1H), 7.24 (m, 2H), 7.35 (m, 2H), 7.47 (m, 1H), 7.56 (m, 1H), 7.73 (s, 1H), 7.79 (s, 1H), 7.86 (d, 1H), 8.67 (s, 1H); Mass spectrum: MH⁺ 564.

The N-{3-chloro-4-[(3-fluorobenzyl)oxy]phenyl}-6-[(piperidin-4-yl)oxy]quinazolin-4-amine used as a starting material was made as follows:

2.6 M hydrogen chloride in ether (10 ml, 26 mmol) was added to a solution of tert-butyl 4-[(4-chloroquinazolin-6-yl)oxy]piperidine-1-carboxylate (2.25 g, 6.45 mmol, prepared as described in example 1, preparation of starting materials) and 3-chloro-4-[(3-fluorobenzyl)oxy]aniline (1.6 g, 6.45 mmol, PCT Int. Appl. WO03/40108, AstraZeneca, Reference example 8.1) in acetonitrile (50 ml). The mixture was heated at 70° C. for 2 hours and cooled to room temperature. The mixture was concentrated under vacuum and partitioned between water and dichloromethane. The solution was basified to pH 11 by addition of aqueous ammonia and extracted with dichloromethane twice. The organic layers were combined, washed with water and dried over magnesium sulfate. After evaporation of the solvents, the residue was purified by chromatography on silica gel (eluant: 10% methanol in dichloromethane, then 10% to 15% 7N methanolic ammonia in dichloromethane) to give N-{3-chloro-4-[(3-fluorobenzyl)oxy]phenyl}-6-[(piperidin-4-yl)oxy]quinazolin-4-amine (620 mg, 22%). NMR Spectrum: (DMSOd₆) 1.53 (m, 2H), 2.00 (m, 2H), 2.63 (m, 2H), 2.99 (m, 2H), 4.64 (m, 1H), 5.26 (s, 2H), 7.19 (m, 1H), 7.27-7.34 (m, 3H), 7.47 (m, 1H), 7.53 (d, 1H), 7.70 (m, 2H), 7.90 (s, 1H), 7.98 (s, 1H), 8.47 (s, 1H), 9.54 (s, 1H); Mass spectrum: MH⁺ 479

### EXAMPLE 8

### N-[3-chloropyridin-2-ylmethoxy)phenyl]-6-({1-[(dimethylamino)acetyl]piperidin-4-yl}oxy)quinazolin-4-amine

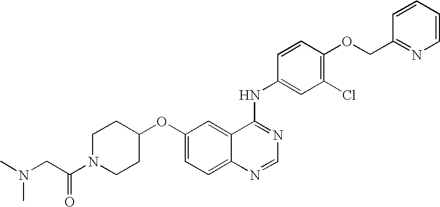

The procedure in example 7 was repeated, except using N-[3-chloro-4-(pyridin-2-ylmethoxy)phenyl]-6-[(piperidin-4-yl)oxy]quinazolin-4-amine (97 mg, 0.21 mmol, prepared as described in example 1, preparation of starting materials) to give the title compound (29 mg, 26%); NMR Spectrum: (CDCl₃) 1.7-2.0 (m, 4H), 2.24 (s, 6H), 3.11 (m, 2H), 3.45-3.75 (m, 2H), 3.7-3.85 (m, 2H), 4.65 (m, 1H), 5.27 (s, 2H), 6.98 (d, 1H), 7.25 (m, 1H), 7.44 (d, 1H), 7.52 (m, 2H), 7.64 (d, 1H), 7.7-7.9 (m, 3H), 8.35 (br s, 1H), 8.58 (br d, 1H), 8.65 (s, 1H); Mass spectrum: MH⁺ 547.

### EXAMPLE 9

### N-[3-chloro-4-(pyrazin-2-ylmethoxy)phenyl]-6-({1-[(dimethylamino)acetyl]piperidin-4-yl}oxy)quinazolin-4-amine

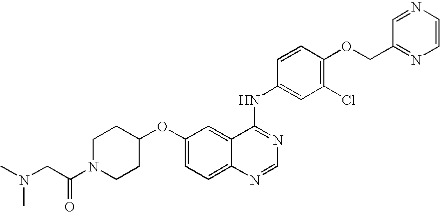

5N hydrogen chloride in isopropanol (63 μl, 0.31 mmol) was added to 4-chloro-6-({1-[(dimethylamino)acetyl]piperidin-4-yl}oxy)quinazoline (100 mg, 0.29 mmol), 3-chloro-4-(pyrazin-2-ylmethoxy)aniline (68 mg, 0.29 mmol) in isopropanol (1 ml). The mixture was stirred at 80° C. for 90 minutes. After cooling, the precipitate was filtered, rinsed with isopropanol and purified on an HPLC column (C18, 5 microns, 19 mm diameter, 100 mm length) of a preparative HPLC-MS system eluting with a mixture of water (containing 5% methanol and 1% acetic acid) and acetonitrile (gradient). After concentration under vacuum, the residue was partitioned between aqueous ammonia and dichloromethane. The organic layer was dried over magnesium sulfate and concentrated to give the title compound as a solid (59 mg, 37%); NMR Spectrum: (CDCl₃) 1.8-2.0 (m, 4H), 2.28 (s, 6H), 3.13 (s, 2H), 3.4-3.7 (m, 2H), 3.8-3.9 (m, 2H), 4.76 (m, 1H), 5.32 (s, 2H), 7.06 (d, 1H), 7.43 (d, 1H), 7.73 (m, 2H), 7.82 (m, 2H), 8.57 (s, 2H), 8.62 (s, 1H), 8.92 (s, 1H), 8.97 (s, 1H); Mass spectrum: MH⁺ 548.

The 3-chloro-4-(pyrazin-2-ylmethoxy)aniline used as starting material was made as follows:

Powdered potassium hydroxide (3.4 g, 60 mmol) was added to a mixture of 2-chloro-1-fluoro-4-nitrobenzene (10.5 g, 60 mmol) and pyrazin-2-ylmethanol (6.6 g, 60 mmol; Maury G. et al., Bull. Soc. Chem. Belg. 1982, 91, 153). Tetrabutylammonium bromide (50 mg) was added and the mixture was heated at 80° C. for one hour and cooled to room temperature. The residue was dissolved in dichloromethane, washed with water and dried over magnesium sulfate. After evaporation of the solvents, the residue was purified by chromatography on silica gel (eluant: 5% ethyl acetate in dichloromethane) to give 2-[(2-chloro-4-nitrophenyl)oxymethyl]pyrazine (6.4 g, 40%) as a yellow solid. NMR Spectrum: (CDCl₃) 5.41 (s, 2H), 7.14 (d, 1H), 8.18 (dd, 1H), 8.35 (d, 1H), 8.61 (d, 2H), 8.94 (s, 1H).

A mixture of 2-[(2-chloro-4-nitrophenyl)oxymethyl]pyrazine (6.4 g, 24 mmol) and platinum oxide (400 mg) in ethyl acetate was stirred at room temperature under hydrogen (atmospheric pressure) for 2 hours. After filtration of the catalyst and evaporation of the solvent under vacuum, the residue was purified by chromatography on silica gel (eluant: 60% ethyl acetate in petroleum ether) to give 3-chloro-4-(pyrazin-2-ylmethoxy)aniline (5 g, 90%). NMR Spectrum: (CDCl₃) 3.53 (s br, 2H), 5.20 (s, 2H), 6.53 (dd, 1H), 6.78 (d, 1H), 6.84 (d, 1H), 8.54 (s, 2H), 8.95 (s, 1H).

The 3-chloro-4-(pyrazin-2-ylmethoxy)aniline used as starting material can also be made by an alternative procedure as follows:

Pyrazin-2-ylmethanol (1.5 g) was dissolved in DMA (25 ml) and the solution was cooled to 0° C. 60% Sodium hydride dispersion in oil (0.6 g) was added portionwise and the mixture was stirred for 10 minutes at 0° C. A solution of 3-chloro-4-fluoronitrobenzene (2.18 g) in DMA (25 ml) was added over 15 minutes and the reaction mixture was allowed to warm to room temperature and stirred for 3 hours. Saturated ammonium chloride (100 ml) was added, and the precipitated solid was filtered off and purified by chromatography eluting with 50% ethyl acetate/iso-hexane. The appropriate fractions were concentrated to give 3-chloro-4-(2-pyrazinylmethoxy)nitrobenzene as a brown solid (1.25 g, 38%).

A solution of 3-chloro-4-(2-pyrazinylmethoxy)nitrobenzene (1.25 g) in ethyl acetate (100 ml) was catalytically hydrogenated over 10% platinum on carbon (400 mg) at ambient temperature overnight. The reaction mixture was filtered through diatomaceous earth and the filtrate was concentrated to give 3-chloro-4-(2-pyrazinylmethoxy)aniline as a yellow solid (1.03 g, 94%).

The 4-chloro-6-({1-[(dimethylamino)acetyl]piperidin-4-yl}oxy)quinazoline used as starting material was made as follows:

Chloroacetyl chloride (1.2 ml, 15 mol) was added dropwise to a biphasic solution of 4-hydroxypiperidine (1 g, 10 mmol) in ethyl acetate (150 ml) and saturated aqueous sodium carbonate (75 ml). The mixture was stirred for 2 hours at room temperature. The organic layer was separated and dried over magnesium sulfate to give 1-chloroacetyl-4-hydroxypiperidine (1.5 g, 84%) after evaporation of the solvents. Mass spectrum: MH⁺ 178.

1-Chloroacetyl-4-hydroxypiperidine (1.5 g, 8.4 mmol) and 2M dimethylamine in THF (13 ml, 25.3 mmol) were stirred at room temperature for one hour. The mixture was diluted with diethyl ether. After filtration, the etheral solution was evaporated under vacuum to give 1-dimethylaminoacetyl-4-hydroxypiperidine (1.45 g, 93%) as an oil which solidified. Mass spectrum: MH⁺ 187.

4-Chloroquinazolin-6-ol (900 mg, 4.8 mmol) in dichloromethane (40 ml) was treated with triphenylphosphine (1.6 g, 6 mmol), 1-dimethylaminoacetyl-4-hydroxypiperidine (900 mg, 4.8 mmol) and di-tert-butylazadicarboxylate (1.4 g, 6 mmol) and stirred under nitrogen for 20 hours. The solution was purified by chromatography using 0 to 2% methanolic ammonia in dichloromethane as eluant to give 4-chloro-6-({1-[(dimethylamino)acetyl]piperidin-4-yl}oxy)quinazoline (1.09 g, 78%) as a white solid; Mass spectrum MH⁺ 349.

### EXAMPLE 10

Using a similar procedure to that described in Example 9 the appropriate 4-chloroquinazoline was reacted with the appropriate aniline in IPA and hydrogen chloride, except that following the reaction with the aniline the product was isolated and washed with IPA and diethylether to give the compounds shown in Table I as dihydrochloride salts:

TABLE I

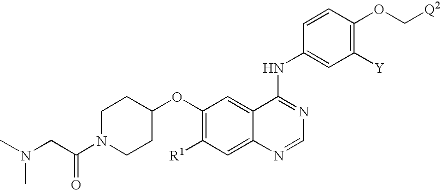

| No. | | | |
|---|---|---|---|
| & | | | |
| Note | R¹ | Y | Q² |
| [1] | hydrogen | methoxy | 3-fluorophenyl |
| [2] | hydrogen | hydrogen | 2-pyridyl |
| [3] | hydrogen | methoxy | 2-pyridyl |
| [4] | methoxy | hydrogen | 3-fluorophenyl |
| [5] | methoxy | methoxy | 3-fluorophenyl |
| [6] | methoxy | chloro | 3-fluorophenyl |
| [7] | methoxy | hydrogen | 2-pyridyl |
| [8] | methoxy | methoxy | 2-pyridyl |
| [9] | methoxy | chloro | 2-pyridyl |
| [10] | methoxy | chloro | 2-pyrazinyl |
| [11] | methoxy | hydrogen | 2-pyrazinyl |
| [12] | methoxy | methoxy | 2-pyrazinyl |

Notes:
[1] 6-({1-[(dimethylamino)acetyl]piperidin-4-yl}oxy)-N-{4-[(3-fluorobenzyl)oxy]-3-methoxyphenyl}quinazolin-4-amine (129 mg, 75%); NMR Spectrum: (DMSOd₆) 1.64(m, 1H), 1.74(m, 1H), 2.09(m, 1H), 2.16(m, 1H), 2.83(s, 6H), 3.2-3.45(m, 2H), 3.60(m, 1H), 3.81(s, 3H), 3.99(m, 1H), 4.34(s, 2H), 5.15(m, 1H), 5.18(s, 2H), 7.12(d, 1H), 7.18(m, 1H), 7.31(m, 3H), 7.46(m, 2H), 7.72(d, 1H), 7.89(d, 1H), 8.73(s, 1H), 8.80(s, 1H), 9.59 (m, 1H); Mass spectrum:
MH⁺ 560.
The 4-[(3-fluorobenzyl)oxy]-3-methoxyaniline used as the starting material was prepared using the procedure described in WO99/35146, page 64; Mass spectrumMH⁺ 248.
[2] 6-({1-[(dimethylamino)acetyl]piperidin-4-yl}oxy)-N-[4-(pyridin-2-ylmethoxy)phenyl]quinazolin-4-amine(116 mg, 71%); NMR Spectrum: (DMSOd₆) 1.64(m, 1H), 1.74(m, 1H), 2.09(m, 1H), 2.17(m, 1H), 2.83(s, 6H), 3.2-3.45(m, 2H), 3.60(m, 1H), 3.99(m, 1H), 4.34(s, 2H), 5.15(m, 1H), 5.26(s, 2H), 7.12(d, 2H), 7.39(m, 1H), 7.58(m, 1H), 7.62(m, 2H), 7.72(d, 1H), 7.89(m, 2H), 8.62(m, 1H), 8.75(s, 1H), 8.80(s, 1H), 9.6(m, 1H); Mass spectrum: MH⁺ 513.
The 4-(pyridin-2-ylmethoxy)aniline starting material was prepared using the procedure described in Bromidge S. et al., Bioorg. Med. Chem. Lett. 2000, 10, 1867.
[3] 6-({1-[(dimethylamino)acetyl]piperidin-4-yl}oxy)-N-[3-methoxy-4-(pyridin-2-ylmethoxy)phenyl]quinazolin-4-amine(135 mg, 78%); NMR Spectrum: (DMSOd₆) 1.65(m, 1H), 1.76(m, 1H), 2.08(m, 1H), 2.15(m, 1H), 2.83(s, 6H), 3.2-3.45(m, 2H), 3.58(m, 1H), 3.82(s, 3H), 3.97(m, 1H), 4.33(s, 2H), 5.09(m, 1H), 5.22(s, 2H), 7.13(d, 2H), 7.30(d, 1H), 7.3(m, 1H), 7.47(s, 1H), 7.57(d, 1H), 7.72(d, 1H), 7.87(m, 2H), 8.60(s, 1H), 8.79(s, 1H), 9.55(m, 1H); Mass spectrum:
MH⁺ 543.
The 3-methoxy-4-(pyridin-2-ylmethoxy)aniline starting material was prepared as follows:
2-picolyl chloride hydrochloride(5.2 g, 32 minol) in anhydrous DMF(80 ml) was added to a suspension of 2-methoxy-4-nitrophenol(4.9 g, 29 mmol) and potassium carbonate (11.9 g, 86 mmol). The mixture was stirred at 100° C. for 3 hours, cooled to room temperature and poured into water. The resulting precipitate was filtered, washed with water and diethyl ether and dried under high vacuum to give 2-methoxy-4-nitro-1-(pyridin-2-ylmethoxy)benzene(7 g, 93%). Mass spectrum: MH⁺ 261
12N hydrochloric acid(8 ml), then tin(II) chloride(8 g, 42 mmol) was added to a solution of 2-methoxy-4-nitro-1-(pyridin-2-ylmethoxy)benzene(2.3 g, 9 mmol) in methanol (35 ml). The mixture was heated at 95° C. for 5 hours. The cooled reaction mixture was diluted with water and neutralised with solid potassium carbonate. Ethyl acetate was added with rapid stirring. The resulting mixture was filtered through a pad of celite. The filtrate was extracted with ethyl acetate. The organic layer was washed
with brine, dried over magnesium sulfate and concentrated under reduced pressure to give 3-methoxy-4-(pyridin-2-ylmethoxy)aniline(1.46 g, 70%) as a brown oil. Mass spectrum: MH⁺ 231.
[4] 6-({1-[(dimethylamino)acetyl]piperidin-4-yl}oxy)-N-{4-[(3-fluorobenzyl)oxy]phenyl}-7-methoxyquinazolin-4-amine(106 mg, 71%); NMR Spectrum: (DMSOd₆) 1.64(m, 1H), 1.77(m, 1H), 2.08-2.17(m, 2H), 2.82(s, 6H), 3.1-3.3(m, 2H), 3.57 (m, 1H), 3.97(m, 1H), 3.98(s, 3H), 4.33(s, 2H), 5.17(m, 1H), 5.19(s, 2H), 7.12(d, 2H), 7.19(m, 1H), 7.32(m, 3H), 7.45(m, 1H), 7.63(d, 2H), 8.72(s, 1H), 8.75(s, 1H), 9.57(m, 1H); Mass spectrum: MH⁺ 560.
The 4-(3-fluorobenzyloxy)aniline was prepared using the procedure described in WO98/02434, page 45.
The 4-chloro-6-({1-[(dimethylamino)acetyl]piperidin-4-yl}oxy)-7-methoxyquinazoline starting material was prepared as follows:
A suspension of 4-chloro-7-methoxyquinazolin-6-yl acetate(prepared as described in Example 25-5 of WO01/66099, 10.1 g, 40 mmol) in 6N methanolic ammonia(200 ml) was stirred at room temperature for 90 minutes. The solvents were evaporated under vacuum. Water was added and the resulting suspension was filtered. The solid obtained was washed with water, ether and dried under high vacuum in the presence of phosphorus pentoxide to give 4-chloro-7-methoxyquinazolin-6-ol(7.9 g, 94%). NMR Spectrum:
(DMSOd₆) 4.02(s, 3H), 7.40(s, 1H), 7.43(s, 1H), 8.81(s, 1H).
Di-tert-butylazadicarboxylate(759 mg, 3.3 mmol) was added portionwise to an ice-cooled solution of 4-chloro-7-methoxyquinazolin-6-ol(462 mg, 2.2 mmol), 1-dimethylaminoacetyl-4-hydroxypiperidine(490 mg, 2.6 mmol, prepared as described in example 9, preparation of starting materials) and triphenyiphosphine(865 mg, 3.3 mmol) in dichloromethane(20 ml). The mixture was stirred at room temperature for 1 hour. After evaporation of the solvent under vacuum, the residue was purified by chromatography on silica
gel(eluant: 0% to 2% 7N methanolic ammonia in dichloromethane) to give 4-chloro-6-({1-[(dimethylamino)acetyl]piperidin-4-yl}oxy)-7-methoxyquinazoline(804 mg, 94%). NMR Spectrum: (CDCl₃) 1.90-2.15(m, 4H), 2.29(s, 6H), 3.15(s, 2H), 3.60-3.70(m, 2H), 3.90(m, 2H), 4.05(s, 3H), 4.81(m, 1H), 7.36(s, 1H), 7.45(s, 1H), 8.87(s, 1H); Mass spectrum: MH⁺ 379.
[5] 6-({1-[(dimethylamino)acetyl]piperidin-4-yl}oxy)-N-{4-[(3-fluorobenzyl)oxy]-3-methoxyphenyl}-7-methoxyquinazolin-4-amine(110 mg, 70%);
NMR Spectrum: (DMSOd₆) 1.64(m, 1H), 1.77(m, 1H), 2.08-2.17(m, 2H), 2.82(s, 6H), 3.2-3.45(m, 2H), 3.57(m, 1H), 3.81(s, 3H), 3.97(m, 1H), 3.99(s, 3H), 4.33(s, 2H), 5.13(m, 1H), 5.17(s, 2H), 7.11(d, 2H), 7.19(m, 1H), 7.25-7.32(m, 3H), 7.46(m, 2H), 8.64(s, 1H), 8.72(s, 1H), 9.56(m, 1H); Mass spectrum: MH⁺ 590.
[6] N-{3-chloro-4-[(3-fluorobenzyl)oxy]phenyl}-6-({1-[(dimethylamino)acetyl]piperidin-4-yl}oxy)-7-methoxyquinazolin-4-amine(116 mg, 73%); NMR Spectrum: (DMSOd₆) 1.65(m, 1H), 1.77(m, 1H), 2.08-2.17(m, 2H), 2.82(s, 6H), 3.2-3.45(m, 2H), 3.57(m, 1H), 3.97(m, 1H), 3.99(s, 3H), 4.33(s, 2H), 5.12(m, 1H), 5.30(s, 2H), 7.19(m, 1H), 7.33(m, 4H), 7.48(m, 1H), 7.70(m, 1H), 7.92(s, 1H), 8.66(m, 1H), 8.79(s, 1H), 9.54(m, 1H); Mass spectrum:
MH⁺ 594.
The 3-chloro-4-[(3-fluorobenzyl)oxy]aniline starting material was prepared as described in example 7, preparation of starting materials.
[7] 6-({1-[(dimethylamino)acetyl]piperidin-4-yl}oxy)-7-methoxy-N-[4-(pyridin-2-ylmethoxy)phenyl]quinazolin-4-amine(110 mg, 75%); NMR Spectrum: (DMSOd₆) 1.63(m, 1H), 1.76(m, 1H), 2.08-2.17(m, 2H), 2.83(s, 6H), 3.2-3.45(m, 2H), 3.58(m, 1H), 3.98(s, 3H), 4.01(m, 1H), 4.34(s, 2H), 5.20(m, 1H), 5.24(s, 2H), 7.12(d, 2H), 7.38(m, 2H), 7.56(d, 1H), 7.65(d, 2H), 7.88(m, 1H), 8.61(d, 1H), 8.75(s, 1H), 8.82(m, 1H), 9.60(m, 1H); Mass spectrum:
MH⁺ 543.
[8] 6-({1-[(dimethylanimo)acetyl]piperidin-4-yl}oxy)-7-methoxy-N-[3-methoxy-4-(pyridin-2-ylmethoxy)phenyl]quinazolin-4-amine(120 mg, 78%); NMR Spectrum: (DMSOd₆) 1.65(m, 1H), 1.77(m, 1H), 2.08-2.17(m, 2H), 2.83(s, 6H), 3.2-3.45(m, 2H), 3.57(m, 1H), 3.81(s, 3H), 3.99(s, 3H), 4.00(m, 1H), 4.33(s, 2H), 5.14(m, 1H), 5.22(s, 2H), 7.11(d, 1H), 7.26(d, 1H), 7.33(s, 1H), 7.38(m, 1H), 7.45(s, 1H), 7.56(d, 1H), 7.88(m, 1H), 8.60(d, 1H), 8.67(s, 1H), 8.76(s, 1H), 9.60
(m, 1H); Mass spectrum: MH⁺ 573.
[9] N-[3-chloro-4-(pyridin-2-ylmethoxy)phenyl]-6-({1-[(dimethylamino)acetyl]piperidin-4-yl}oxy)-7-methoxyquinazolin-4-amine(107 mg, 69%); NMR Spectrum: (DMSOd₆) 1.63(m, 1H), 1.76(m, 1H), 2.08-2.18(m, 2H), 2.83(s, 6H), 3.2-3.45(m, 2H), 3.58(m, 1H), 3.99(s, 3H), 4.02(m, 1H), 4.33(s, 2H), 5.20(m, 1H), 5.34(s, 2H), 7.34(m, 2H), 7.39(m, 1H), 7.60(d, 1H), 7.71(dd, 1H), 7.90(m, 1H), 7.95(s, 1H), 8.62(d, 1H), 8.81(s, 2H), 9.57(m, 1H); Mass spectrum:
MH⁺ 577.
The 3-chloro-4-(pyridin-2-ylmethoxy)aniline starting material was prepared as described in example 1, preparation of starting materials.
[10] N-[3-chloro-4-(pyrazin-2-ylmethoxy)phenyl]-6-({1-[(dimethylamino)acetyl]piperidin-4-yl}oxy)-7-methoxyquinazolin-4-amine(120 mg, 78%); NMR Spectrum: (DMSOd₆) 1.63(m, 1H), 1.76(m, 1H), 2.08-2.18(m, 2H), 2.83(s, 6H), 3.2-3.45(m, 2H), 3.59(m, 1H), 3.99(s, 3H), 4.02(m, 1H), 4.33(s, 2H), 5.20(m, 1H), 5.43(s, 2H), 7.35(s, 1H), 7.40(d, 1H), 7.75(d, 1H), 7.97(s, 1H), 8.67(s, 1H), 8.71(s, 1H), 8.81(s, 2H), 8.87(s, 1H), 9.57(m, 1H); Mass spectrum:
MH⁺ 578.
The 3-chloro-4-(pyrazin-2-ylmethoxy)aniline starting material was prepared as described in Example 9, preparation of starting materials.
[11] 6-({1-[(dimethylamino)acetyl]piperidin-4-yl}oxy)-7-methoxy-N-[4-(pyrazin-2-ylmethoxy)phenyl]quinazolin-4-amine(63 mg, 65%); NMR Spectrum: (DMSOd₆) 1.66(m, 1H), 1.78(m, 1H), 2.08-2.18(m, 2H), 2.83(s, 6H), 3.2-3.45(m, 2H), 3.59(m, 1H), 3.95(m, 1H), 3.99(s, 3H), 4.32(s, 2H), 5.10(m, 1H), 5.33(s, 2H), 7.17(d, 2H), 7.31(s, 1H), 7.63(d, 2H), 8.61(s br, 1H), 8.66(s, 1H), 8.70(s, 1H), 8.75(s, 1H), 8.85(s, 1H), 9.55(m, 1H); Mass spectrum:
MH⁺ 544.
The 4-(pyrazin-2-ylmethoxy)aniline used as starting material was prepared by reacting 4-fluoro-1-nitrobenzene and pyrazin-2-ylmethanol using an analogous procedure to that described in example 9 for the preparation of 3-chloro-4-(pyrazin-2-ylmethoxy)aniline, to give 2-(4-nitrophenoxymethyl)pyrazine [(100 mg, 43%); NMR Spectrum: (CDCl₃) 5.34(s, 2H), 7.10(d, 2H), 8.24(d, 2H), 8.60(s, 2H), 8.82(s, 1H)], which was then reduced under hydrogen in the presence of a platinum oxide
catalyst using the procedure described in example 9, preparation of starting materials to give 4-(pyrazin-2-ylmethoxy)aniline [73 mg, 85%, Mass spectrum: MH⁺ 202]
[12] 6-({1-[(dimethylamino)acetyl]piperidin-4-yl}oxy)-7-methoxy-N-[3-methoxy-4-(pyrazin-2-ylmethoxy)phenyl]quinazolin-4-amine(71 mg, 61%); NMR Spectrum: (DMSOd₆) 1.65(m, 1H), 1.77(m, 1H), 2.08-2.18(m, 2H), 2.83(s, 6H), 3.2-3.45(m, 2H), 3.58(m, 1H), 3.81(s, 3H), 3.99(s, 3H), 4.00(m, 1H), 4.33(s, 2H), 5.16(m, 1H), 5.30(s, 2H), 7.17(d, 1H), 7.29(d, 1H), 7.34(s, 1H), 7.48(s, 1H), 8.66-8.69(m, 3H), 8.78(s, 1H), 8.83(s, 1H), 9.55(m, 1H);
Mass spectrum: MH⁺ 574.

The 3-methoxy-4-(pyrazin-2-ylmethoxy)aniline starting material was obtained as follows:

Di-tert-butylazadicarboxylate (272 mg, 1.2 mmol) and pyrazin-2-ylmethanol (130 mg, 1.2 mmol) were added successively to an ice-cooled mixture of 2-methoxy-4-nitrophenol (200 mg, 1.2 mmol) and triphenylphosphine (310 mg, 1.2 mmol) in dichloromethane (6 ml). The mixture was stirred at room temperature for 1 hour. After evaporation of the solvent under vacuum, the residue was purified by chromatography on silica gel (eluant: 10%: 10% up to 40%: 40% ethyl acetate-dichloromethane in petroleum ether) to give 2-[(2-methoxy-4-nitrophenoxy)methyl]pyrazine containing triphenylphosphine oxide (282 mg): Mass spectrum: MH⁺ 262

2-[(2-methoxy-4-nitrophenoxy)methyl]pyrazine was reduced by hydrogenation in the presence of platinum oxide using an analogous procedure to that described in example 9, preparation of starting materials to give 3-methoxy-4-(pyrazin-2-ylmethoxy)aniline (270 mg containing 62% wt triphenylphosphine oxide; 94%; Mass spectrum: MH⁺ 232).

### EXAMPLE 11

### 6-({1-[(dimethylamino)acetyl]piperidin-4-yl}oxy)-N-[4-(3-fluorobenzyloxy)phenyl]quinazolin-4-amine

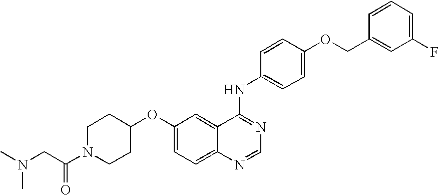

Di-tert-butylazadicarboxylate (92 mg, 0.4 mmol) was added to a mixture of 6-({1-[(dimethylamino)acetyl]piperidin-4-yl}oxy)-N-(4-hydroxyphenyl)quinazolin-4-amine (84 mg, 0.19 mmol), 3-fluorobenzyl alcohol (26 μl, 0.24 mmol) and triphenyl phosphine (104 mg, 0.4 mmol) in dichloromethane (2 ml). After stirring for one hour, more di-tert-butylazadicarboxylate (45 mg, 0.2 mmol), 3-fluorobenzyl alcohol (26 μl, 0.24 mmol) and triphenyl phosphine (55 mg, 0.2 mmol) were added to complete the reaction. After 1 hour, the mixture was evaporated under vacuum and purified by chromatography on silica gel (eluant: 2% 7N methanolic ammonia in dichloromethane) to give the title compound (30 mg, 30%). NMR Spectrum: (CDCl₃) 1.90 (m, 2H), 2.02 (m, 2H), 2.29 (s, 6H), 3.14 (s, 2H), 3.60 (m, 1H), 3.68 (m, 1H), 3.86 (m, 2H), 4.70 (m, 1H), 5.09 (s, 2H), 7.02 (m, 3H), 7.20 (m, 4H), 7.36 (dd, 1H), 7.47 (dd, 1H), 7.57 (d, 2H), 7.87 (d, 1H), 8.65 (s, 1H); Mass spectrum: MH⁺ 530

The 6-({1-[(dimethylamino)acetyl]piperidin-4-yl}oxy)-N-(4-hydroxyphenyl)quinazolin-4-amine was prepared as follows:

4-chloro-6-({1-[(dimethylamino)acetyl]piperidin-4-yl}oxy)quinazoline (prepared as described in example 10, preparation of starting materials) was reacted with 4-hydroxyaniline in IPA and HCl using an analogous procedure to that described in example 10 to give 6-({1-[(dimethylamino)acetyl]piperidin-4-yl}oxy)-N-(4-hydroxyphenyl)quinazolin-4-amine as the dihydrochloride salt. The dihydrochloride salt was then dissolved in 5% 7N methanolic ammonia in dichloromethane, filtered, evaporation of the filtrate and trituration of the residue in diethyl ether to give 6-({1-[(dimethylamino)acetyl]piperidin-4-yl}oxy)-N-(4-hydroxyphenyl)quinazolin-4-amine (86 mg, 62%); Mass spectrum: MH⁺ 422.

### EXAMPLE 12

### 6-({1-[(dimethylamino)acetyl]piperidin-4-yl}oxy)-N-[3-methoxy-4-(pyrazin-2-ylmethoxy)phenyl]quinazolin-4-amine

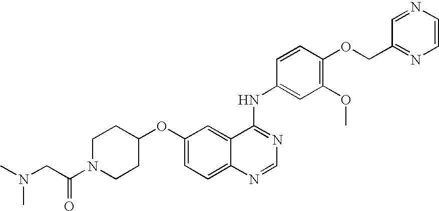

A solution of freshly prepared pyrazin-2-ylmethyl methanesulfonate (90 mg, 0.48 mmol; prepared according to the procedure described in Piera et al., An. Quim., 1979, 75, 899) in dimethylacetamide (2 ml) was added to 6-({1-[(dimethylamino)acetyl]piperidin-4-yl}oxy)-N-(4-hydroxy-3-methoxyphenyl)quinazolin-4-amine dihydrochloride (168 mg, 0.32 mmol) and potassium carbonate (220 mg, 1.6 mmol). The mixture was stirred at room temperature for 18 hours. After filtration, the mixture was injected on an HPLC column (C18, 5 microns, 19 mm diameter, 100 mm length) of a preparative HPLC-MS system eluting with a mixture of water (containing 5% methanol and 1% acetic acid) and acetonitrile (gradient). After evaporation of the solvents, the residue was repurified by chromatography on silica gel (eluant: 5% 7N methanolic ammonia in dichloromethane) to give the title compound as the free base (17 mg, 10%); NMR Spectrum: (CDCl₃) 1.90 (m, 2H), 2.02 (m, 2H), 2.29 (s, 6H), 3.14 (s, 2H), 3.60 (m, 1H), 3.67 (m, 1H), 3.85 (m, 2H), 3.95 (s, 3H), 4.72 (m, 1H), 5.33 (s, 2H), 7.0-7.3 (m, 4H), 7.48 (m, 2H), 7.88 (d, 1H), 8.56 (d, 2H), 8.67 (s, 1H), 8.90 (s, 1H); Mass spectrum: MH⁺ 544.

The 6-({1-[(dimethylamino)acetyl]piperidin-4-yl}oxy)-N-(4-hydroxy-3-methoxyphenyl)quinazolin-4-amine dihydrochloride starting material was prepared as follows:

4-chloro-6-({1-[(dimethylamino)acetyl]piperidin-4-yl}oxy)quinazoline (prepared as described in example 9, preparation of starting materials) was reacted with 4-hydroxy-3-methoxyaniline (prepared as described in Chem. Ber., 1897, 30, 2444) in IPA and HCl, followed by isolation and washing with IPA and diethylether using an analogous procedure to that described in example 10 to give 6-({1-[(dimethylamino)acetyl]piperidin-4-yl}oxy)-N-(4-hydroxy-3-methoxyphenyl)quinazolin-4-amine dihydrochloride (300 mg, 79%, Mass spectrum: MH⁺ 452).

### EXAMPLE 13

### 6-({1-[(dimethylamino)acetyl]piperidin-4-yl}oxy)-N-[4-(pyrazin-2-ylmethoxy)phenyl]quinazolin-4-amine

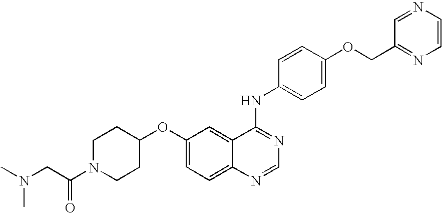

The procedure described in example 12 was repeated using 6-({1-[(dimethylamino)acetyl]piperidin-4-yl}oxy)-N-(4-hydroxyphenyl)quinazolin-4-amine dihydrochloride and pyrazin-2-ylmethyl methanesulfonate to give the title compound (22 mg, 13%); NMR Spectrum: (CDCl₃) 1.89 (m, 2H), 2.02 (m, 2H), 2.29 (s, 6H), 3.14 (s, 2H), 3.61 (m, 1H), 3.68 (m, 1H), 3.84 (m, 2H), 4.71 (m, 1H), 5.28 (s, 2H), 7.08 (d, 2H), 7.17 (s, 1H), 7.20 (s, 1H), 7.47 (d, 1H), 7.61 (d, 2H), 7.87 (d, 1H), 8.57 (d, 2H), 8.65 (s, 1H), 8.86 (s, 1H); Mass spectrum: MH⁺ 514.

The 6-({1-[(dimethylamino)acetyl]piperidin-4-yl}oxy)-N-(4-hydroxyphenyl)quinazolin-4-amine dihydrochloride starting material was prepared as follows:

4-chloro-6-({1-[(dimethylamino)acetyl]piperidin-4-yl}oxy)quinazoline (prepared as described in example 9, preparation of starting materials) was reacted with 4-hydroxyaniline in IPA and HCl, followed by isolation and washing with IPA and diethylether using an analogous procedure to that described in example 9 to give 6-({1-[(dimethylamino)acetyl]piperidin-4-yl}oxy)-N-(4-hydroxyphenyl)quinazolin-4-amine dihydrochloride (325 mg, 91%, Mass spectrum: MH⁺ 422).

### EXAMPLE 14

### N-[3-Chloro-4-(pyridin-2-ylmethoxy)phenyl]-6-{[1-(methylsulfonyl)pyrrolidin-3-yl]methoxy}quinazolin-4-amine

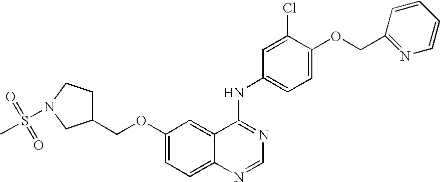

A mixture of N,N-diisopropylethylamine (628 μl), methanesulfonyl chloride (84 μl) and N-[3-chloro-4-(pyridin-2-ylmethoxy)phenyl]-6-(pyrrolidin-3-ylmethoxy)quinazolin-4-amine (166 mg) in DCM (5 ml) was stirred overnight. The solution was concentrated in vacuo and the residue purified by chromatography using ethyl acetate→DCM-5% methanol as eluant to give the title compound as a white solid (85 mg, 44%); NMR spectrum (DMSO-d6) 1.79-1.90 (m, 1H), 2.11-2.20 (m, 1H), 2.76-2.85 (m, 1H), 2.94 (s, 3H), 3.14-3.20 (m, 2H), 3.37-3.45 (m, 1H), 3.50-3.55 (dd, 1H), 4.11-4.17 (dd, 1H), 4.18-4.23 (dd, 1H), 5.30 (s, 2H), 7.29 (d, 1H), 7.38 (dd, 1H), 7.53 (dd, 1H), 7.60 (d, 1H), 7.72 (dd, 1H), 7.74 (d, 1H), 7.86-7.93 (m, 2H), 8.00 (d, 1H), 8.50 (s, 1H), 8.61 (d, 1H) and 9.57 (s, 1H); Mass spectrum MH⁺ 540.

The N-[3-chloro-4-(pyridin-2-ylmethoxy)phenyl]-6-(pyrrolidin-3-ylmethoxy)quinazolin-4-amine used as starting material was prepared as follows:

The procedure described in example 1 (preparation of starting materials) was repeated using 4-chloroquinazolin-6-ol and tert-butyl 3-(hydroxymethyl)pyrrolidine-1-carboxylate to give tert-butyl 3-{[(4-chloroquinazolin-6-yl)oxy]methyl}pyrrolidine-1-carboxylate as a white solid in 46% yield; Mass spectrum MH⁺ 364.

The procedure described in example 1 (preparation of starting materials) was repeated using tert-butyl 3-{[(4-chloroquinazolin-6-yl)oxy]methyl}pyrrolidine-1-carboxylate and 3-chloro-4-(pyridin-2-ylmethoxy)aniline to give N-[3-chloro-4-(pyridin-2-ylmethoxy)phenyl]-6-(pyrrolidin-3-ylmethoxy)quinazolin-4-amine in 56% yield; Mass spectrum MH⁺ 462.

### EXAMPLE 15

### 2-{4-[(4-{[3-Chloro-4-(pyridin-2-ylmethoxy)phenyl]amino}-7-methoxyquinazolin-6-yl)oxy]piperidin-1-yl}-2-oxoethanol

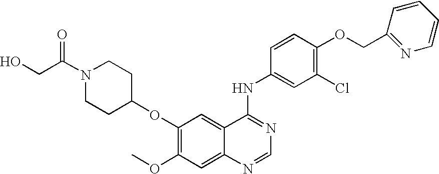

A suspension of N-[3-chloro-4-(pyridin-2-ylmethoxy)phenyl]-7-methoxy-6-(piperidin-4-yloxy)quinazolin-4-amine (300 mg, 0.61 mmol), glycolic acid (46 mg, 0.61 mmol), diisopropylethylamine (0.21 ml, 1.22 mmol) and HATU (278 mg, 0.73 mmol) in dichloromethane (10 ml) was stirred at room temperature. The suspension became homogeneous after 1 hour and a precipitate was formed after 18 hours stirring. The precipitate was filtered and dried under high vacuum to give the title compound (141 mg, 42%) as a pale solid; NMR Spectrum: (DMSO-d6) 1.74-1.65 (m, 2H), 2.00 (m, 2H), 3.40 (m, 2H), 3.61 (m, 1H), 3.82 (m, 1H), 3.93 (s, 3H), 4.13 (d, 2H), 4.55 (m, 1H), 4.79 (m, 1H), 5.30 (s, 2H), 7.22 (s, 1H), 7.28 (d, 1H), 7.37 (m, 1H), 7.59 (d, 1H), 7.67 (m, 1H), 7.94-7.87 (m, 3H), 8.45 (s, 1H), 8.60 (d, 1H), 9.43 (s, 1H); Mass spectrum: MH⁺ 550.

The N-[3-chloro-4-(pyridin-2-ylmethoxy)phenyl]-7-methoxy-6-(piperidin-4-yloxy)quinazolin-4-amine used as starting material was made as follows:

3-Chloro-4-(pyridin-2-ylmethoxy)aniline (598 mg, 2.54 mmol) and 5N hydrogen chloride in isopropanol (0.5 ml, 2.5 mmol) were added to tert-butyl 4-[(4-chloro-7-methoxyquinazolin-6-yl)oxy]piperidine-1-carboxylate (1 g, 40 mmol; prepared as described in Example 16 of WO2003/082831) in isopropanol (10 ml). The mixture was stirred at 80° C. for 90 minutes. After evaporation of the mixture to dryness, the residue was dissolved in DCM (25 ml) and TFA (15 ml). The mixture was stirred at room temperature for 90 minutes. The solvents were evaporated under vacuum and the residue was azeotroped with toluene. 7N ammonia in methanol (5 ml) and DCM (30 ml) was added. After evaporation of the solvents, the residue was purified by chromatography on silica gel (eluant: 6 to 9% 7N ammonia-methanol in DCM) to give N-[3-chloro-4-(pyridin-2-ylmethoxy)phenyl]-7-methoxy-6-(piperidin-4-yloxy)quinazolin-4-amine (897 mg, 72%) as a pale solid; Mass spectrum: MH⁺ 92.

### EXAMPLE 16

### 6-[(1-Acetylpiperidin-4-yl)oxy]-N-[3-chloro-4-(pyridin-2-ylmethoxy)phenyl]-7-methoxyquinazolin-4-amine

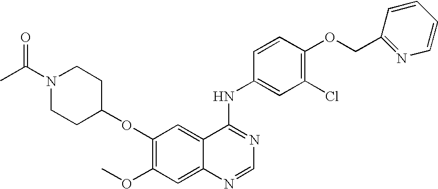

Acetic anhydride (90 μl, 0.91 mmol) was added dropwise to a suspension of N-[3-chloro-4-(pyridin-2-ylmethoxy)phenyl]-7-methoxy-6-(piperidin-4-yloxy)quinazolin-4-amine (300 mg, 0.61 mmol) and potassium carbonate (210 mg, 1.52 mmol) in acetone (10 ml). The mixture was stirred at room temperature for 90 minutes. The solids were filtered off. 7N ammonia in methanol (5 ml) was added and the solvents were evaporated under vacuum. The residue was purified by chromatography on silica gel (eluant: 2 to 5% 7N ammonia-methanol in DCM) to give the title compound (262 mg, 80%) as a pale solid; NMR Spectrum: (CDCl₃) 2.0-1.7 (m, 4H), 2.12 (s, 3H), 3.41 (m, 1H), 3.57 (m, 1H), 3.75 (m, 1H), 3.88 (m, 1H), 3.98 (s, 3H), 4.65 (m, 1H), 5.28 (s, 2H), 6.99 (d, 1H), 7.25 (m, 1H), 7.43 (s, 1H), 7.50 (d, 1H), 7.65 (d, 1H), 7.76 (m, 2H), 7.90 (m, 1H), 8.60 (m, 2H); Mass spectrum: MH⁺ 534.

### EXAMPLE 17

### 2-{4-[(4-{[3-Chloro-4-(pyrazin-2-ylmethoxy)phenyl]amino}-7-methoxyquinazolin-6-yl)oxy]piperidin-1-yl}-2-oxoethanol

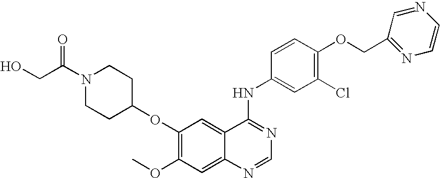

The procedure described in Example 15 was repeated using N-[3-chloro-4-(pyrazin-2-ylmethoxy)phenyl]-7-methoxy-6-(piperidin-4-yloxy)quinazolin-4-amine (300 mg, 0.61 mmol) and glycolic acid (46 mg, 0.61 mg) to give the title compound (215 mg, 64%) as a pale solid; NMR Spectrum: (DMSO-d6) 1.73-1.67 (m, 2H), 2.01 (m, 2H), 3.40 (m, 2H), 3.61 (m, 1H), 3.83 (m, 1H), 3.94 (s, 3H), 4.14 (d, 2H), 4.58 (m, 1H), 4.79 (m, 1H), 5.38 (s, 2H), 7.22 (s, 1H), 7.34 (d, 1H), 7.70 (d, 1H), 7.93 (s, 1H), 7.96 (s, 1H), 8.46 (s, 1H), 8.67 (s, 1H), 8.70 (s, 1H), 8.87 (s, 1H), 9.46 (m, 1H); Mass spectrum: MH⁺ 551.

The N-[3-chloro-4-(pyrazin-2-ylmethoxy)phenyl]-7-methoxy-6-(piperidin-4-yloxy)quinazolin-4-amine used as starting material was prepared from tert-butyl 4-[(4-chloro-7-methoxyquinazolin-6-yl)oxy]piperidine-1-carboxylate (1 g, 2.54 mmol) and 3-chloro-4-(pyrazin-2-ylmethoxy)aniline (5.98 mg, 2.54 mmol) using the route described in Example 15 starting material (1.19 g, 95%); Mass spectrum: MH⁺ 493.

### EXAMPLE 18

### 6-[(1-Acetylpiperidin-4-yl)oxy]-N-[3-chloro-4-(pyrazin-2-ylmethoxy)phenyl]-7-methoxyquinazolin 4-amine

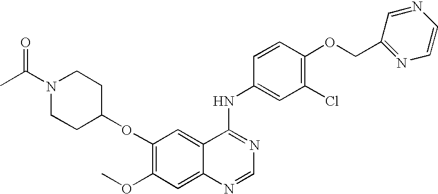

The procedure described in Example 16 was repeated using N-[3-chloro-4-(pyrazin-2-ylmethoxy)phenyl]-7-methoxy-6-(piperidin-4-yloxy)quinazolin-4-amine (300 mg, 0.61 mmol) and acetic anhydride (90 μl, 0.91 mmol) to give the title compound (255 mg, 78%) as a pale solid; NMR Spectrum: (DMSO-d6) 1.63 (m, 1H), 1.73 (m, 1H), 1.96 (m, 1H), 2.04 (s, 3H), 2.05 (m, 1H), 3.40 (m, 2H), 3.70 (m, 1H), 3.81 (m, 1H), 3.94 (s, 3H), 4.78 (m, 1H), 5.38 (s, 2H), 7.22 (s, 1H), 7.35 (d, 1H), 7.70 (d, 1H), 7.92 (s, 1H), 7.96 (s, 1H), 8.46 (s, 1H), 8.67 (s, 1H), 8.70 (s, 1H), 8.87 (s, 1H), 9.42 (m, 1H); Mass spectrum: MH⁺ 535.

### EXAMPLE 19

### 6-[(1-Acetylpiperidin-4-yl)oxy]-N-[3-chloro-4-(pyridin-2-ylmethoxy)phenyl]quinazolin-4-amine

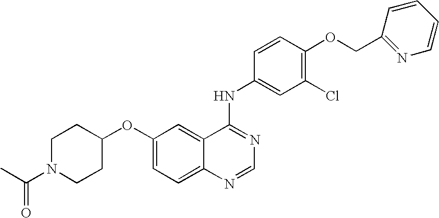

The procedure described in Example 16 was repeated using N-[3-chloro-4-(pyridin-2-ylmethoxy)phenyl]-6-(piperidin-4-yloxy)quinazolin-4-amine (250 mg, 0.54 mmol) and acetic anhydride (77 μl, 0.81 mmol) to give the title compound (171 mg, 63%) as a pale solid; NMR Spectrum: (CDCl₃) 2.0-1.7 (m, 4H), 2.12 (s, 3H), 3.45 (m, 1H), 3.79-3.66 (m, 3H), 4.70 (m, 1H), 5.29 (s, 2H), 7.00 (d, 1H), 7.26 (m, 1H), 7.39 (s, 1H), 7.46 (d, 1H), 7.52 (d, 1H), 7.66 (d, 1H), 7.75 (dd, 1H), 7.81 (s, 1H), 7.87 (d, 1H), 8.59 (s, 1H), 8.66 (s, 1H); Mass spectrum: MH⁺ 504.

### EXAMPLE 20

### 2-{4-[(4-{[3-Chloro-4-(pyrazin-2-ylmethoxy)phenyl]amino}quinazolin-6-yl)oxy]piperidin-1-yl}-2-oxoethanol

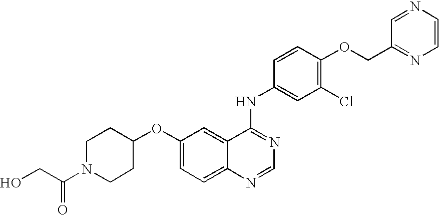

The procedure described in Example 15 was repeated using N-[3-chloro-4-(pyrazin-2-ylmethoxy)phenyl]-6-(piperidin-4-yloxy)quinazolin-4-amine (250 mg, 0.54 mmol) and glycolic acid (41 mg, 0.54 mg) except that at the end of the reaction, the reaction mixture was washed with 5% aqueous sodium bicarbonate and the organic layer was dried over MgSO₄. After evaporation of the solvents, the residue was purified by chromatography on silica gel (eluant: 5 to 7% 7N ammonia-methanol in DCM) to give the title compound (215 mg, 64%) as a pale solid; NMR Spectrum: (CDCl₃+2 drops DMSO-d6) 2.01-1.93 (m, 4H), 3.30 (m, 1H), 3.56 (m, 1H), 3.81 (m, 2H), 4.21 (s, 2H), 4.86 (m, 1H), 5.33 (s, 2H), 7.08 (d, 1H), 7.43 (d, 1H), 7.86-7.74 (m, 4H), 8.58 (s, 2H), 8.62 (s, 1H), 8.97 (s, 2H); Mass spectrum: MH⁺ 521.

The N-[3-chloro-4-(pyrazin-2-ylmethoxy)phenyl]-6-(piperidin-4-yloxy)quinazolin-4-amine used as starting material was prepared from tert-butyl 4-[(4-chloroquinazolin-6-yl)oxy]piperidine-1-carboxylate (1 g, 2.75 mmol) and 3-chloro-4-(pyrazin-2-ylmethoxy)aniline (757 mg, 2.75 mmol) using the procedure described in Example 15 starting material; Mass spectrum: MH⁺ 463.

### EXAMPLE 21

### 6-[(1-Acetylpiperidin-4-yl)oxy]-N-[3-chloro-4-(pyrazin-2-ylmethoxy)phenyl]quinazolin-4-amine

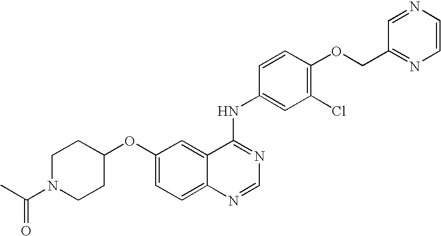

The procedure described in Example 16 was repeated using N-[3-chloro-4-(pyrazin-2-ylmethoxy)phenyl]-6-(piperidin-4-yloxy)quinazolin-4-amine (250 mg, 0.54 mmol) and acetic anhydride (66 μl, 0.70 mmol) to give the title compound (208 mg, 76%) as a pale solid; NMR Spectrum: (CDCl₃) 2.0-1.7 (m, 4H), 2.12 (s, 3H), 3.45 (m, 1H), 3.75-3.65 (m, 3H), 4.69 (m, 1H), 5.32 (s, 2H), 7.04 (d, 1H), 7.45 (m, 2H), 7.62 (d, 1H), 7.80 (s, 1H), 7.86 (d, 1H), 8.19 (s br, 1H), 8.57 (s, 2H), 8.66 (s, 1H), 8.97 (s, 1H); Mass spectrum: MH⁺ 505.

### EXAMPLE 22

### N-[3-Chloro-4-(pyridin-2-ylmethoxy)phenyl]-6-{[1-(methylsulfonyl)piperidin-4-yl]oxy}quinazolin-4-amine

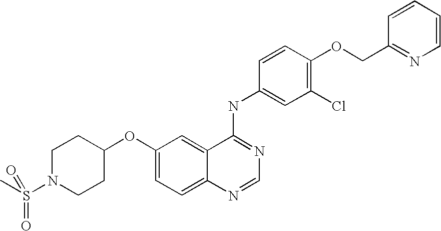

4-Chloro-6-{[1-(methylsulfonyl)piperidin-4-yl]oxy}quinazoline (0.070 g) and 3-chloro-4-(pyridin-2-ylmethoxy)aniline (0.048 g) were heated in EPA (3 ml) containing N,N-diisopropylethylamine (0.101 ml) under reflux for 4 hours. The solution was cooled and a solid filtered off. This was triturated with acetonitrile to give N-[3-chloro-4-(pyridin-2-ylmethoxy)phenyl]-6-{[1-(methylsulfonyl)piperidin-4-yl]oxy}quinazolin-4-amine as a white solid (0.056 g, 53%); Mass spectrum Me 540.

The 4-chloro-6-{[1-(methylsulfonyl)piperidin-4-yl]oxy}quinazoline used as starting material was prepared as follows:

Quinazoline-4,6-diol (3.46 g, prepared as described in J. Med. Chem., 1983, 26, 420) in DMF (200 ml) at 0° C. was treated with sodium hydride (60% in oil) (0.85 g) and stirred for 2 hours at ambient temperature. Pivaloyl chloride (3.82 g) was added at 0° C. and the mixture stirred overnight. The solution was partitioned between EtOAc and saturated aqueous sodium hydrogen carbonate and the organic phase washed with brine and evaporated. The residue was purified by chromatography using ethyl acetate-isohexane as eluant to give (6-hydroxy-4-oxoquinazolin-3(4H)-yl)methyl pivalate (1.21 g, 21%); NMR spectrum (DMSO-d6) 1.14 (s, 9H), 5.93 (s, 2H), 7.26-7.31 (m, 1H), 7.44 (d, 1H), 7.54 (d, 1H), 8.26 (s, 1H), 10.20 (s, 1H); Mass spectrum M⁺ 276.

(6-Hydroxy-4-oxoquinazolin-3(4H)-yl)methyl pivalate (0.878 g) in DCM (35 ml) containing triphenylphosphine (0.96 g) and tert-butyl 4-hydroxypiperidine-1-carboxylate (0.74 g) was treated with di-tert-butylazadicarboxylate (0.84 g) in DCM (5 ml) with cooling and the mixture stirred overnight. The mixture was purified by chromatography using ethyl acetate-isohexane as eluant to give tert-butyl 4-[(3-{[(2,2-dimethylpropanoyl)oxy]methyl}-4-oxo-3,4-dihydroquinazolin-6-yl)oxy]piperidine-1-carboxylate (1.33 g, 91%); Mass spectrum M⁺ 459.

tert-Butyl 4-[(3-{[(2,2-dimethylpropanoyl)oxy]methyl}-4-oxo-3,4-dihydroquinazolin-6-yl)oxy]piperidine-1-carboxylate (1.26 g) in acetonitrile (20 ml) was treated with HCl (4.0M in dioxane) (2.73 ml) and stirred for 1.5 hours. The solution was evaporated and dissolved in DCM (20 ml). Triethylamine (0.76 ml) was added then methanesulfonyl chloride (0.27 ml) and the solution stirred for 1 hour and evaporated. The residue was dissolved in ammonia in methanol (50 ml) and the solution stirred overnight. The mixture was evaporated and the residue purified by chromatography using methanol-DCM as eluant to give 6-{[1-(methylsulfonyl)piperidin-4-yl]oxy}quinazolin-4-ol (0.60 g, 68%); Mass spectrum M⁺ 323.

6-{[1-(Methylsulfonyl)piperidin-4-yl]oxy}quinazolin-4-ol (0.60 g) in thionyl chloride (8 ml) was treated with DMF (0.128 ml) and heated at reflux under nitrogen for 2 hours. The solution was evaporated and azeotroped with toluene to give 4-chloro-6-{[1-(methylsulfonyl)piperidin-4-yl]oxy}quinazoline (0.747 g, 100%).

### EXAMPLE 23

### N-{3-Ethynyl-4-[(3-fluorobenzyl)oxy]phenyl}-7-methoxy-6-{[1-(methylsulfonyl)piperidin-4-yl]oxy}quinazolin-4-amine

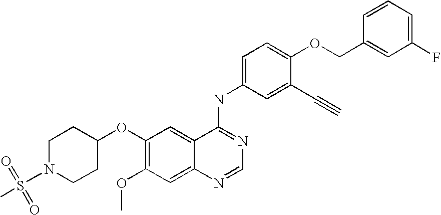

N-{3-Ethynyl-4-[(3-fluorobenzyl)oxy]phenyl}-7-methoxy-6-(piperidin-4-yloxy)quinazolin-4-amine (0.052 g) was suspended in DCM (5 ml) and methanesulfonyl chloride (0.007 ml) added and stirred at ambient temperature for 3 hours. Triethylamine (0.012 ml) followed by methanesulfonyl chloride (0.007 ml) was added and stirred at ambient temperature for a further 20 hours. The solution was filtered and the filtrate evaporated in vacuo. Purification by preparative HPLC gave N-{3-ethynyl-4-[(3-fluorobenzyl)oxy]phenyl}-7-methoxy-6-{[1-(methylsulfonyl)piperidin-4-yl]oxy}quinazolin-4-amine as a white solid (0.0163 g, 31%); Mass spectrum M⁺ 577.

The N-{3-ethynyl-4-[(3-fluorobenzyl)oxy]phenyl}-7-methoxy-6-(piperidin-4-yloxy)quinazolin-4-amine used as starting material was made as follows:

tert-Butyl 4-[(4-chloro-7-methoxyquinazolin-6-yl)oxy]piperidine-1-carboxylate (0.122 g) and 3-ethynyl-4-[(3-fluorobenzyl)oxy]aniline (0.075 g, prepared as described in reference example 30.1 of WO2003/04010) were heated in IPA (5 ml) containing 2.0 M HCl in ether (2 ml) under reflux for 4 hours. The solution was cooled and a solid filtered off to give the title compound. (0.116 g, 75%); NMR spectrum (DMSO-d6) 1.9 (m, 2H), 2.3 (m, 2H), 3.2 (m, 2H), 3.3 (m, 2H), 4.0 (s, 3H), 4.4 (s, 1H), 5.1 (m, 1H), 5.3 (s, 2H), 7.2 (t, 1H), 7.2 (d, 1H), 7.31-7.33 (m, 3H), 7.5 (q, 1H), 7.7 (d, 1H), 7.8 (d, 1H), 8.7 (s, 1H), 8.8 (s, 1H); Mass spectrum M⁺ 499.

### EXAMPLE 24

### 7-Methoxy-6-{[1-(methylsulfonyl)piperidin-4-yl]oxy}-N-[4-(1,3-thiazol-2-ylthio)phenyl]quinazolin-4-amine

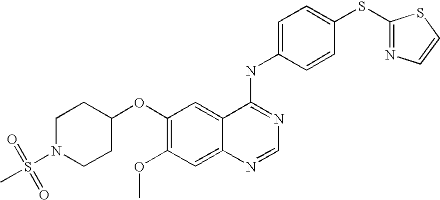

The procedure described in Example 23 was repeated using 7-methoxy-6-(piperidin-4-yloxy)-N-[4-(1,3-thiazol-2-ylthio)phenyl]quinazolin-4-amine (0.020 g) to give 7-methoxy-6-{[1-(methylsulfonyl)piperidin-4-yl]oxy}-N-[4-(1,3-thiazol-2-ylthio)phenyl]quinazolin-4-amine as a white solid (0.0212 g, 98%); Mass spectrum M⁺ 544.

The 7-methoxy-6-(piperidin-4-yloxy)-N-[4-(1,3-thiazol-2-ylthio)phenyl]quinazolin-4-amine used as starting material was made according to procedure in Example 23, starting material using tert-butyl 4-[(4-chloro-7-methoxyquinazolin-6-yl)oxy]piperidine-1-carboxylate and 4-(1,3-thiazol-2-ylthio)aniline; 0.050 g, 21%; NMR spectrum (DMSO-d6); 1.9 (m, 2H), 2.3 (m, 2H), 3.2 (m, 2H), 3.3 (m, 2H), 4.0 (s, 3H), 5.2 (m, 1H), 7.4 (s, 1H), 7.71 (d, 1H), 7.74 (d, 2H), 7.8 (d, 1H), 7.97 (d, 2H), 8.8 (m, 1H), 8.86 (m, 1H), 8.90 (s, 1H), 8.92 (s, 1H); Mass spectrum M⁻ 464.

The 4-(1,3-thiazol-2-ylthio)aniline used as starting material was prepared as follows (see also example 10 of U.S. Pat. No. 3,679,695):

The procedure described in the alternative procedure for making 3-chloro-4-(pyrazin-2-ylmethoxy)aniline in Example 9 was repeated using 1-fluoro-4-nitrobenzene and 1,3-thiazole-2-thiol to give 2-[(4-nitrophenyl)thio]-1,3-thiazole in 68% yield and 4-(1,3-thiazol-2-ylthio)aniline in 84% yield; Mass spectrum M⁺ 209.

### EXAMPLE 25

### N-[3-Chloro-4-(pyrazin-2-ylmethoxy)phenyl]-6-{[1-(methylsulfonyl)piperidin-4-yl]oxy}quinazolin-4-amine

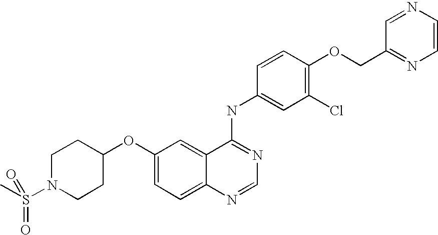

The procedure described in Example 22 was repeated using 4-chloro-6-{[1-(methylsulfonyl)piperidin-4-yl]oxy}quinazoline (0.107 g) and 3-chloro-4-(pyrazin-2-ylmethoxy)aniline (0.089 g) to give the title compound as white crystals in 24% yield; NMR spectrum (DMSO-d6) 1.79-1.90 (m, 2H), 2.09-2.19 (m, 2H), 2.94 (s, 3H), 3.18-3.26 (m, 2H), 3.37-3.44 (m, 2H), 4.91-4.98 (m, 1H), 5.47 (s, 2H), 7.44 (d, 1H), 7.68-7.72 (m, 1H), 7.75-7.79 (m, 1H), 7.89 (d, 1H), 7.95 (d, 1H), 8.43-8.47 (m, 1H), 8.67-8.73 (m, 2H), 8.88 (d, 2H), 10.52 (s, 1H), 11.48-11.57 (m, 1H); Mass spectrum M⁺ 541.

### EXAMPLE 26

### N-{3-Fluoro-4-[(1-methyl-1H-imidazol-2-yl)thio]phenyl}-6-{[1-(methylsulfonyl)piperidin-4-yl]oxy}quinazolin-4-amine

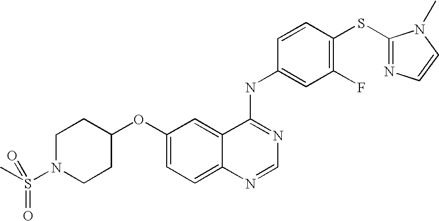

The procedure described in Example 22 was repeated using 4-chloro-6-{[1-(methylsulfonyl)piperidin-4-yl]oxy}quinazoline and 3-fluoro-4-[(1-methyl-1H-imidazol-2-yl)thio]aniline (prepared as described in example 6.2 of WO2003040108) to give the title compound as white crystals in 57% yield; NMR spectrum (DMSO-d6) 1.75-1.86 (m, 2H), 2.09-2.18 (m, 2H), 2.96 (s, 3H), 3.18-3.27 (m, 2H), 3.38-3.46 (m, 2H), 3.87 (s, 3H), 5.05-5.12 (m, 1H), 7.53-7.60 (m, 1H), 7.69 (s, 1H), 7.76-7.88 (m, 3H), 7.95 (d, 1H), 8.04-8.09 (m, 1H), 8.80 (s, 1H), 8.93 (s, 1H), 12.04 (s, 1H); Mass spectrum M⁺ 529.

### EXAMPLE 27

### N-{3-Chloro-4-[(1-methyl-1-imidazol-2-yl)thio]phenyl}-6-{[1-(methylsulfonyl)piperidin-4-yl]oxy}quinazolin-4-amine

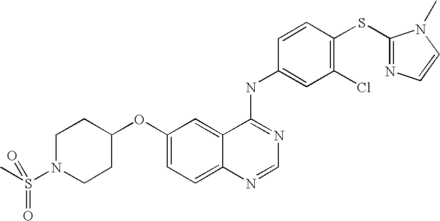

The procedure described in Example 22 was repeated using 4-chloro-6-{[1-(methylsulfonyl)piperidin-4-yl]oxy}quinazoline and 3-chloro-4-[(1-methyl-1H-imidazol-2-yl)thio]aniline (prepared as described in example 10 of WO-96/15118) to give the title compound as white crystals in 68% yield; NMR spectrum (DMSO-d6) 1.74-1.86 (m, 2H), 2.10-2.18 (m, 2H), 2.95 (s, 3H), 3.19-3.27 (m, 2H), 3.39-3.46 (m, 2H), 3.86 (s, 3H), 5.07-5.14 (m, 1), 7.20 (d, 1H), 7.75 (s, 1H), 7.76-7.81 (m, 1H), 7.88-8.00 (m, 3H), 8.24 (d, 1H), 8.86 (d, 1H), 8.95 (s, 1H), 12.28 (s, 1H); Mass spectrum M⁺ 547.

### EXAMPLE 28

### 6-{[1-(Methylsulfonyl)piperidin-4-yl]oxy}-N-[4-(1,3-thiazol-2-ylthio)phenyl]quinazol-4-amine

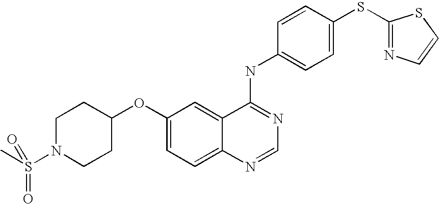

The procedure described in Example 22 was repeated using 4-chloro-6-{[1-(methylsulfonyl)piperidin-4-yl]oxy}quinazoline and 4-(1,3-thiazol-2-ylthio)aniline (prepared as described in Example 24) to give the title compound as white crystals in 63% yield; NMR spectrum (DMSO-d6) 1.80-1.90 (m, 2H), 2.09-2.19 (m, 2H), 2.97 (s, 3H), 3.19-3.28 (m, H), 3.37-3.45 (m, 2H), 4.96-5.03 (m, 1H), 7.73 (d, 1H), 7.75-7.79 (m, 2H), 7.80-7.82 (m, 2H), 7.91-7.96 (m, 3H), 8.54-8.57 (m, 1H), 8.94 (s, 1H), 11.76 (s, 1H); Mass spectrum M⁺ 514.

### EXAMPLE 29

### N-{3-Fluoro-4-[(1-methyl-1H-imidazol-2-yl)thio]phenyl}-7-methoxy-6-{[1-(methylsulfonyl)piperidin-4-yl]oxy}quinazolin-4-amine

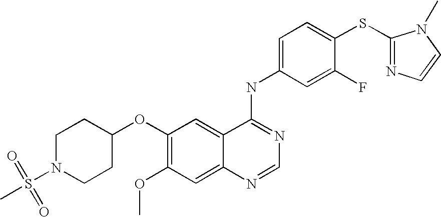

The procedure described in Example 23 was repeated using N-{3-fluoro-4-[(1-methyl-1H-imidazol-2-yl)thio]phenyl}-7-methoxy-6-(piperidin-4-yloxy)quinazolin-4-amine to give N-{3-fluoro-4-[(1-methyl-1H-imidazol-2-yl)thio]phenyl}-7-methoxy-6-{[1-(methylsulfonyl)piperidin-4-yl]oxy}quinazolin-4-amine as a white solid (0.0488 g, 34%); Mass spectrum M⁻ 557.

The N-{3-fluoro-4-[(1-methyl-1H-imidazol-2-yl)thio]phenyl}-7-methoxy-6-(piperidin-4-yloxy)quinazolin-4-amine used as starting material was made following procedure described in Example 23 starting material using tert-butyl 4-[(4-chloro-7-methoxyquinazolin-6-yl)oxy]piperidine-1-carboxylate and 3-fluoro-4-[(1-methyl-1H-imidazol-2-yl)thio]aniline (prepared as described in reference example 6.2 of WO2003040108) to give N-{3-fluoro-4-[(1-methyl-1H-imidazol-2-yl)thio]phenyl}-7-methoxy-6-(piperidin-4-yloxy)quinazolin-4-amine (0.293 g, quant); NMR spectrum (DMSO-d6); 1.9 (m, 2H), 2.3 (m, 2H), 3.2 (m, 2H), 3.3 (m, 2H), 3.8 (s, 3H), 4.0 (s, 3H), 5.3 (m, 1H), 7.48 (s, 1H), 7.70 (d, 0.5H), 7.75 (d, 1H), 7.80 (d, 0.5H), 7.87 (d, 1H), 7.95 (dd, 1H), 8.10 (dd, 1H), 8.90 (s, 1H), 9.0 (m, 1H), 9.14 (s, 1H), 9.17 (m, 1H); Mass spectrum M⁺ 481.

### EXAMPLE 30

### 2-(4-{[4-({3-methyl-4-[(6-methylpyridin-3-yl)oxy]phenyl}amino)quinazolin-6-yl]oxy}piperidin-1-yl)-2-oxoethanol

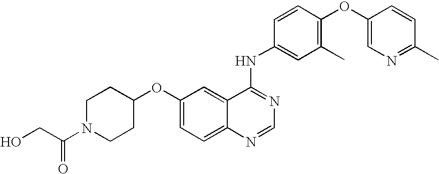

Acetoxyacetyl chloride (98 μl, 0.91 mmol) was added dropwise to an ice-cooled solution of N-{3-methyl-4-[(6-methylpyridin-3-yl)oxy]phenyl}-6-(piperidin-4-yloxy)quinazolin-4-amine (366 mg, 0.83 mmol) and triethylamine (138 W, 0.99 mmol) in DCM (10 ml). The mixture was stirred at room temperature for 2 hours. After evaporation of the mixture to dryness, pyrrolidine (0.68 ml, 8.3 mmol) was added and the mixture was stirred at 65° C. for 2 hours. After cooling and evaporation of the solvents, the residue was purified on an HPLC column (C18, 5 microns, 19 mm diameter, 100 mm length) of a preparative HPLC-MS system eluting with a mixture of water (containing 5% methanol and 1% acetic acid) and acetonitrile (gradient). The combined fractions were evaporated under vacuum. The residue was diluted in aqueous ammonia and extracted with DCM. The organic layer was dried over magnesium sulfate to give the title compound (172 mg, 41%) as a pale solid. NMR spectrum (CDCl₃) 2.00-1.90 (m, 4H), 2.27 (s, 3H), 2.53 (s, 3H), 3.26 (m, 1H), 3.53 (m, 1H), 3.84-3.75 (m, 2H), 4.20 (s, 2H), 4.78 (m, 1H), 6.89 (d, 1H), 7.10 (d, 1H), 7.16 (d, 1H), 7.39 (s, 1H), 7.47 (m, 2H), 7.59 (s, 1H), 7.74 (m, 1H), 7.89 (d, 1H), 8.22 (s, 1H), 8.67 (s, 1H); Mass spectrum: MH⁺ 500.

The N-{3-methyl-4-[(6-methylpyridin-3-yl)oxy]phenyl}-6-(piperidin-4-yloxy)quinazolin-4-amine used as starting material was made as follows:

Sodium hydride (25.6 g, 60% dispersion in oil, 0.64 mol) was added portionwise to a solution of 5-hydroxy-2-methylpyridine (70 g, 0.64 mol) in DMA (700 ml) while keeping the temperature below 40° C. At the end of the addition, the mixture was stirred at room temperature for 1 hour and 2-fluoro-5-nitrotoluene (91.3 g, 0.59 mol) in DMA (100 ml) was added slowly. The mixture was stirred at 80° C. for 3 hours and then cooled. The solvents were evaporated under vacuum and the residue was partitioned between ethyl acetate and water. The organic layer was washed with water and brine and then dried over MgSO₄. After evaporation of the solvents, the residue was purified by chromatography on silica gel (eluant: 30% ethyl acetate in petroleum ether) to give 2-methyl-5-(2-methyl-4-nitrophenoxy)pyridine (141 g, 98%) as an oil; NMR spectrum (CDCl₃); 2.43 (s, 3H), 2.59 (s, 3H), 6.74 (d, 1H), 7.21 (d, 1H), 7.27 (d, 1H), 8.00 (d, 1H), 8.17 (s, 1H), 8.32 (s, 1H).

A mixture of 2-methyl-5-(2-methyl-4-nitrophenoxy)pyridine (141 g, 0.58 mol) and 10% palladium on charcoal (13 g) in ethyl acetate (200 ml) and ethanol (700 ml) was stirred under an atmosphere of hydrogen (1.2 bar) for 5 hours. After reaction completion, the mixture was purged with nitrogen and the catalyst was filtered off. The filtrate was evaporated to dryness to give 3-methyl-4-[(6-methylpyridin-3-yl)oxy]aniline (120.6 g, 98%) as a white solid; Mass spectrum MH⁺ 215.

3-Methyl-4-[(6-methylpyridin-3-yl)oxy]aniline was coupled to tert-butyl 4-[(4-chloroquinazolin-6-yl)oxy]piperidine-1-carboxylate using the procedure described in Example 15 starting material to give N-{3-methyl-4-[(6-methylpyridin-3-yl)oxy]phenyl}-6-(piperidin-4-yloxy)quinazolin-4-amine (412 mg, 93%); Mass spectrum: MH⁺ 442.

### EXAMPLE 31

### 2-{3-[(4-{[3-chloro-4-(pyridin-2-ylmethoxy)phenyl]amino}quinazolin-6-yl)oxy]azetidin-1-yl}-2-oxoethanol

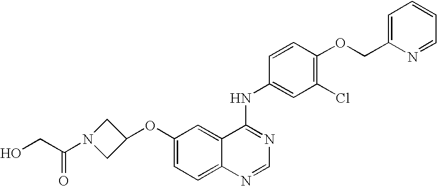

The procedure described in Example 30 was repeated using 6-(azetidin-3-yloxy)-N-[3-chloro-4-(pyridin-2-ylmethoxy)phenyl]quinazolin-4-amine (279 mg, 0.64 mmol) and acetoxyacetyl chloride, except that diisopropylethylamine was used instead of triethylamine and that the acetoxy deprotection step was performed at 45° C. for 2 hours rather than 65° C. After evaporation of the solvents, the mixture was triturated in dichloromethane to give the title compound (234 mg, 74%) as a pale solid; NMR Spectrum: (DMSOd₆) 3.92 (m, 1H), 3.97 (d, 2H), 4.22 (m, 1H), 4.50 (m, 1H), 4.77 (m, 1H), 5.05 (t, 1H), 5.25 (m, 1H), 5.31 (s, 2H), 7.29 (d, 1H), 7.38 (m, 1H), 7.50 (m, 1H), 7.59 (d, 1H), 7.68 (m, 2H), 7.77 (d, 1H), 7.89 (m, 1H), 7.96 (s, 1H), 8.50 (s, 1H), 8.60 (s, 1H), 9.60 (s br, 1H); Mass spectrum: MH⁺ 492.

The 6-(azetidin-3-yloxy)-N-[3-chloro-4-(pyridin-2-ylmethoxy)phenyl]quinazolin-4-amine used as starting material was made as follows:

Hydrogen chloride in dioxane (4M, 69 ml, 274 mmol) was added to a solution of 4-chloroquinazolin-6-yl acetate (15.3 g, 69 mmol) and 3-chloro-4-(pyridin-2-ylmethoxy)aniline (17.7 g, 75 mmol) in acetonitrile (580 ml) heated in an oil bath at 100° C. The mixture was refluxed for 4 hours. After cooling, the solvents were evaporated under vacuum. The residue was taken into 7N ammonia-methanol (100 ml) and the mixture stirred at room temperature for 1.5 hours. The solvents were evaporated under vacuum. The residue was triturated with water. The resulting solid was filtered and dried under high vacuum to give 4-{[3-chloro-4-(pyridin-2-ylmethoxy)phenyl]amino}quinazolin-6-ol (25.2 g, 97%) as a solid; Mass spectrum: MH⁺ 1379.

Di-tert-butyl azadicarboxylate (485 mg, 2.11 mmol) was added portionwise to triphenylphosphine (553 mg, 2.11 mmol) in THF (10 ml) cooled at −20° C. The mixture was stirred for 15 minutes at −20° C. 1-tert-Butoxycarbonyl-4-hydroxyazetidine (219 mg, 1.26 mmol; prepared as described in Falgueyret, J. P., J. Med. Chem, 2001, 44, 94) was added portionwise and the mixture was stirred for 15 minutes at −20° C. 4-{[3-Chloro-4-(pyridin-2-ylmethoxy)phenyl]amino}quinazolin-6-ol (400 mg, 1.05 mmol) was added and the mixture was heated to 70° C. for 24 hours. After cooling, the solvents were evaporated under vacuum and the residue was purified by chromatography on silica gel (eluant: 2 to 5% 7N ammonia-methanol in dichloromethane) to give tert-butyl 3-[(4-{[3-chloro-4-(pyridin-2-ylmethoxy)phenyl]amino}quinazolin-6-yl)oxy]azetidine-1-carboxylate (413 mg, 73%) as a solid; Mass spectrum: MH⁺ 534.

Tert-Butyl 3-[(4-{[3-chloro-4-(pyridin-2-ylmethoxy)phenyl]amino}quinazolin-6-yl)oxy]azetidine-1-carboxylate (413 mg, 0.77 mmol) in DCM (5 ml)-trifluoroacetic acid (5 ml) was stirred at room temperature for 75 minutes. The solvents were evaporated under vacuum. The residue was dissolved in DCM. This was washed with aqueous ammonia, dried over magnesium sulfate and concentrated to dryness to give 6-(azetidin-3-yloxy)-N-[3-chloro-4-(pyridin-2-ylmethoxy)phenyl]quinazolin-4-amine (270 mg, 80%); Mass spectrum: MH⁺ 434.

### EXAMPLE 32

### 2-(3-{[4-({3-methyl-4-[(6-methylpyridin-3-yl)oxy]phenyl}amino)quinazolin-6-yl]oxy}azetidin-1-yl)-2-oxoethanol

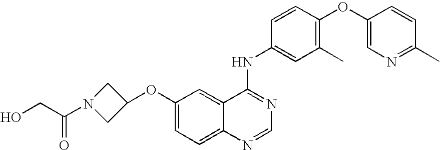

The procedure described in Example 30 was repeated using 6-(azetidin-3-yloxy)-N-{3-methyl-4-[(6-methylpyridin-3-yl)oxy]phenyl}quinazolin-4-amine (300 mg, 0.72 mmol) and acetoxyacetyl chloride to give the title compound (94 mg, 27%) as a pale solid, except that diisopropylethylamine was used instead of triethylamine and that the acetoxy deprotection step was performed at 60° C. for 2 hours rather than 65° C.; NMR Spectrum: (DMSOd₆) 2.23 (s, 3H), 2.44 (s, 3H), 3.92 (m, 1H), 3.97 (d, 2H), 4.23 (m, 1H), 4.50 (m, 1H), 4.77 (m, 1H), 5.05 (t, 1H), 5.24 (m, 1H), 6.99 (d, 1H), 7.24 (m, 2H), 7.50 (m, 1H), 7.78-7.65 (m, 4H), 8.18 (s, 1H), 8.50 (s, 1H), 9.60 (s br, 1H); Mass spectrum: MH⁺ 472.

The 6-(azetidin-3-yloxy)-N-{3-methyl-4-[(6-methylpyridin-3-yl)oxy]phenyl}quinazolin-4-amine used as starting material was made as follows:

3-Methyl-4-[(6-methylpyridin-3-yl)oxy]aniline was coupled to 4-chloroquinazolin-6-yl acetate using the procedure described in Example 31 starting material to give 4-({3-methyl-4-[(6-methylpyridin-3-yl)oxy]phenyl}amino)quinazolin-6-ol (8.4 g, quantitative); Mass spectrum: MH⁺ 359.

4-({3-Methyl-4-[(6-methylpyridin-3-yl)oxy]phenyl}amino)quinazolin-6-ol and 1-tert-butoxycarbonyl-4-hydroxyazetidine were coupled under Mitsunobu conditions (using procedure described in Example 31 starting material) to give tert-butyl 3-{[4-({3-methyl-4-[(6-methylpyridin-3-yl)oxy]phenyl}amino)quinazolin-6-yl]oxy}azetidine-1-carboxylate (946 mg, 67%); Mass spectrum MH⁺ 514.

Tert-Butyl 3-{[4-({3-methyl-4-[(6-methylpyridin-3-yl)oxy]phenyl}amino)quinazolin-6-yl]oxy}azetidine-1-carboxylate was deprotected (using procedure described in Example 31 starting material) to give 6-(azetidin-3-yloxy)-N-{3-methyl-4-[(6-methylpyridin-3-yl)oxy]phenyl}quinazolin-4-amine (653 mg, 86%); Mass spectrum MH⁺ 414.

1. A quinazoline derivative of the Formula I: 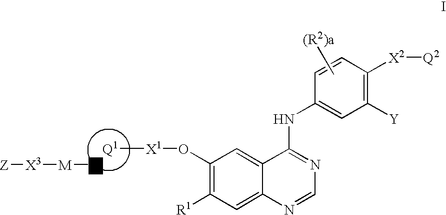
wherein:
R¹ is selected from hydrogen, hydroxy, (1-6C)alkoxy, (3-7C)cycloalkyl-oxy and (3-7C)cycloalkyl-(1-6C)alkoxy,
and wherein adjacent carbon atoms in any (2-6C)alkylene chain within a R¹ substituent are optionally separated by the insertion into the chain of a group selected from O, S, SO, SO₂, N(R³), CO, CON(R³), N(R³)CO, SO₂N(R³) and N(R³)SO₂, wherein R³ is hydrogen or (1-6C)alkyl,
and wherein any CH₂ or CH₃ group within a R¹ substituent optionally bears on each said CH₂ or CH₃ group one or more halogeno or (1-6C)alkyl substituents, or a substituent selected from hydroxy, cyano, amino, carboxy, carbamoyl, sulfamoyl, oxo, thioxo, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino;
Y is selected from hydrogen, halogeno, (1-4C)alkyl, (1-4C)alkoxy, (2-4C)alkenyl and (2-4C)alkynyl;
a is 0, 1, 2 or 3 or 4;
each R², which may be the same or different, is selected from halogeno, (1-4C)alkyl, (1-4C)alkoxy, (2-4C)alkenyl and (2-4C)alkynyl;
X² is a direct bond or is selected from O, S, OC(R⁴)₂, SC(R⁴)₂, SO, SO₂, N(R⁴), CO and N(R⁴)C(R⁴)₂ wherein each R⁴ is, which may be the same or different, is selected from hydrogen or (1-6C)alkyl, and Q² is aryl or heteroaryl,
and wherein Q² optionally bears one or more substituents (for example 1, 2 or 3), which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, and a group of the formula:
—X⁴—R⁵
wherein X⁴ is a direct bond or is selected from O, CO and N(R⁶), wherein R⁶ is hydrogen or (1-6C)alkyl, and R⁵ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N-di-[(1-6C)alkyl]carbamoyl-(1-6C)alkyl, sulfamoyl(1-6C)alkyl, N-(1-6C)alkylsulfamoyl(1-6C)alkyl, N,N di-(1-6C)alkylsulfamoyl(1-6C)alkyl, (2-6C)alkanoyl-(1-6C)alkyl, (2-6C)alkanoyloxy-(1-6C)alkyl or (1-6C)alkoxycarbonyl-(1-6C)alkyl,
and wherein any CH₂ or CH₃ group within —X²-Q² optionally bears on each said CH₂ or CH₃ one or more (for example 1, 2, or 3) halogeno or (1-6C)alkyl substituents or a substituent selected from hydroxy, cyano, amino, (1-4C)alkoxy, (1-4C)alkylamino and di-[(1-4C)alkylamino];
X¹ is a direct bond or C(R⁷)₂, wherein each R⁷, which may be the same or different, is selected from hydrogen and (1-4C)alkyl;
ring Q¹ is a 4, 5, 6 or 7 membered saturated or partially unsaturated heterocyclyl group containing 1 nitrogen heteroatom and optionally 1 or 2 additional heteroatoms selected from O, S and N, and which ring is linked to the group X¹ by a ring carbon;
M is selected from CO and SO₂;
X³ is a group of the formula:
—(CR⁸R⁹)ₚ-(Q³)ₘ-(CR¹⁰R¹¹)_{q}—
wherein m is 0 or 1, p is 0, 1, 2, 3 or 4 and q is 0, 1, 2, 3 or 4,
each of R⁸, R⁹, R¹⁰ and R¹¹, which may be the same or different, is selected from hydrogen and (1-6C)alkyl, and
Q³ is selected from (3-7C)cycloalkylene and (3-7C)cycloalkenylene;
Z is selected from hydrogen, hydroxy, amino, (1-6C)alkylamino, di-[(1-6C)alkyl]amino, (1-6C)alkoxy, (1-6C)alkylsulfonyl, (1-6C)alkanesulfonylamino, N-(1-6C)alkyl-(1-6C)alkanesulfonylamino, and a group of the formula:
Q⁴-X⁵—
wherein X⁵ is a direct bond or is selected from O, N(R²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,
provided that when X⁵ is a direct bond, Q⁴ is heterocyclyl,
and provided that when m, p and q are all 0, then Z is heterocyclyl,
and wherein adjacent carbon atoms in any (2-6C)alkylene chain within a Z substituent are optionally separated by the insertion into the chain of a group selected from O, S, SO, SO₂, N(R¹³), CO, —C═C— and —C≡C— wherein R¹³ is hydrogen or (1-6C)alkyl,
and wherein any CH₂ or CH₃ group within any Z, X¹ or X³ group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
and wherein any heterocyclyl group represented by Q¹ or within a Z substituent optionally bears one or more (for example 1, 2 or 3) substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴
wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,
and wherein any heterocyclyl group represented by Q¹ or within a Z substituent optionally bears 1 or 2 oxo or thioxo substituents;
or a pharmaceutically acceptable salt thereof.

2. A quinazoline derivative of the Formula I as defined in claim 1, wherein R¹ is selected from hydrogen, hydroxy and (1-6C)alkoxy,
and wherein adjacent carbon atoms in any (2-6C)alkylene chain within a R¹ substituent are optionally separated by the insertion into the chain of a group selected from O, S, SO, SO₂, N(R³), CO, CON(R³), N(R³)CO, SO₂N(R³) and N(R³)SO₂, wherein R³ is hydrogen or (1-6C)alkyl,
and wherein any CH₂ or CH₃ group within a R¹ substituent optionally bears on each said CH₂ or CH₃ group one or more halogeno or (1-6C)alkyl substituents, or a substituent selected from hydroxy, cyano, amino, carboxy, carbamoyl, sulfamoyl, oxo, thioxo, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-1-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino.

3. A quinazoline derivative of the Formula I as defined in claim 1, wherein when X² is CO or SO, then M is not CO.

4. A quinazoline derivative of the Formula I as defined in claim 1 or 3, wherein R¹ is selected from hydrogen, (1-6C)alkoxy, cyclopropyl-(1-4C)alkoxy, cyclobutyl-(1-4C)alkoxy, cyclopentyl-(1-4C)alkoxy and cyclohexyl-(1-6C)alkoxy,
and wherein any CH₂ or CH₃ group within a R¹ substituent optionally bears on each said CH₂ or CH₃ group one or more fluoro or chloro substituents, or a substituent selected from hydroxy, methoxy and ethoxy.

5. A quinazoline derivative of the Formula I as defined in claim 4, wherein R¹ is selected from hydrogen, methoxy, ethoxy, propyloxy, isopropyloxy, cyclopropylmethoxy, 2-hydroxyethoxy, 2-fluoroethoxy, 2-methoxyethoxy, 2-ethoxyethoxy, 2,2-difluoroethoxy and 2,2,2-trifluoroethoxy.

6. A quinazoline derivative of the Formula I as defined in claim 4, wherein R¹ is selected from hydrogen and (1-3C)alkoxy;

7. A quinazoline derivative of the Formula I as defined in claim 6, wherein R¹ is hydrogen.

8. A quinazoline derivative of the Formula I as defined in claim 6, wherein R¹ is methoxy.

9. A quinazoline derivative of the Formula I as defined in any one of the preceding claims, wherein Y is selected from hydrogen, halogeno, (1-4C)alkyl, (1-4C)alkoxy and (2-4C)alkynyl.

10. A quinazoline derivative of the Formula I as defined in claim 9, wherein Y is selected from hydrogen, fluoro, chloro, methyl, methoxy and ethynyl.

11. A quinazoline derivative of the Formula I as defined in claim 9, wherein Y is halogeno.

12. A quinazoline derivative of the Formula I as defined in any one of the preceding claims, wherein a is 0.

13. A quinazoline derivative of the Formula I as defined in any one of the preceding claims, wherein X² is selected from O, S and OC(R⁴)₂ wherein each R⁴ is, independently, hydrogen or (1-4C)alkyl.

14. A quinazoline derivative of the Formula I as defined in any one of the preceding claims, wherein X² is selected from O, S and OCH₂.

15. A quinazoline derivative of the Formula I as defined in claim 14, wherein X² is O.

16. A quinazoline derivative of the Formula I as defined in claim 14, wherein X² is S.

17. A quinazoline derivative of the Formula I as defined in claim 14, wherein X² is OCH₂;

18. A quinazoline derivative of the Formula I as defined in any one of the preceding claims, wherein Q² is selected from phenyl and a 5- or 6-membered monocyclic heteroaryl ring, which ring contains 1, 2 or 3 heteroatoms independently selected from oxygen, nitrogen and sulfur,
and wherein Q² optionally bears one or more substituents, which may be the same or different, selected from halogeno, cyano, nitro, hydroxy, amino, carboxy, carbamoyl, sulfamoyl, formyl, mercapto, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-1-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, and a group of the formula:
—X⁴—R⁵—
wherein X⁴ is a direct bond or is selected from O, CO and N(R⁶), wherein R⁶ is hydrogen or (1-6C)alkyl, and R⁵ is halogeno-(1-6C)alkyl, hydroxy-(1-6C)alkyl, carboxy-(1-6C)alkyl, (1-6C)alkoxy-(1-6C)alkyl, cyano-(1-6C)alkyl, amino-(1-6C)alkyl, N-(1-6C)alkylamino-(1-6C)alkyl, N,N-di-[(1-6C)alkyl]amino-(1-6C)alkyl, (2-6C)alkanoylamino-(1-6C)alkyl, N-(1-6C)alkyl-(2-6C)alkanoylamino-(1-6C)alkyl, (1-6C)alkoxycarbonylamino-(1-6C)alkyl, carbamoyl-(1-6C)alkyl, N-(1-6C)alkylcarbamoyl-(1-6C)alkyl, N,N di-[(1-6C)alkyl]carbamoyl-(1-6C)alkyl, sulfamoyl(1-6C)alkyl, N-(1-6C)alkylsulfamoyl(1-6C)alkyl, N,N di-(1-6C)alkylsulfamoyl(1-6C)alkyl, (2-6C)alkanoyl-(1-6C)alkyl, (2-6C)alkanoyloxy-(1-6C)alkyl or (1-6C)alkoxycarbonyl-(1-6C)alkyl,
and wherein any CH₂ or CH₃ group within Q² optionally-bears on each said CH₂ or CH₃ one or more halogeno or (1-6C)alkyl substituents or a substituent selected from hydroxy, cyano, amino, (1-4C)alkoxy, (1-4C)alkylamino and di-[(1-4C)alkylamino].

19. A quinazoline derivative of the Formula I as defined in any one of the preceding claims, wherein Q² is selected from phenyl, pyridyl, pyrazinyl, 1,3-thiazolyl, 1H-imidazolyl, 1H-pyrazolyl, 1,3-oxazolyl and isoxazolyl,
and wherein Q² optionally bears one or more substituents, which may be the same or different, as hereinbefore defined claim 18.

20. A quinazoline derivative of the Formula I as defined in any one of the preceding claims, wherein Q² is selected from phenyl, pyridyl, pyrazinyl, 1,3-thiazolyl and 1H-imidazolyl,
and wherein Q² optionally bears one or more substituents, which may be the same or different, as hereinbefore defined in claim 18.

21. A quinazoline derivative of the Formula I as defined in any one of the preceding claims, wherein Q² is selected from phenyl, 2-pyridyl and 2-pyrazinyl,
and wherein Q² optionally bears 1, 2, or 3 substituents, which may be the same or different, selected from fluoro, chloro, hydroxy, cyano, nitro, (1-4C)alkyl and (1-4C)alkoxy.

22. A quinazoline derivative of the Formula I as defined in any one of the preceding claims, wherein Q² is selected from 2-pyridyl, 6-methyl-pyrid-3yl, 3-fluorophenyl, 2-pyrazinyl, 1,3-thiazol-2-yl and 1-methyl-1H-imidazol-2-yl.

23. A quinazoline derivative of the Formula I as defined in any one of the preceding claims, wherein X¹ is selected from a direct bond and CH₂.

24. A quinazoline derivative of the Formula I as defined in any one of the preceding claims, wherein Q¹ is selected from azetidinyl, pyrrolidinyl, piperidinyl, homopiperidinyl, piperazinyl, morpholinyl and thiomorpholinyl,
and wherein Q¹ is linked to the group X¹—O by a ring carbon atom,
and wherein Q¹ optionally bears one or more substituents, which may be the same or different, selected from halogeno, trifluoromethyl, hydroxy, carbamoyl, (1-4C)alkyl, (1-4C)alkoxy, N-(1-4C)alkylcarbamoyl and N,N-di-[(1-4C)alkyl]carbamoyl,
and wherein any heterocyclyl group within Q¹ optionally bears an oxo substituent.

25. A quinazoline derivative of the Formula I as defined in any one of the preceding claims, wherein Q¹ is selected from azetidinyl, pyrrolidinyl and piperidinyl,
and wherein Q¹ is linked to the group X¹—O by a ring carbon atom,
and wherein Q¹ optionally bears one or more substituents, which may be the same or different, selected from halogeno, trifluoromethyl, hydroxy, carbamoyl, (1-4C)alkyl, (1-4C)alkoxy, N-(1-4C)alkylcarbamoyl and N,N-di-[(1-4C)alkyl]carbamoyl,
and wherein any heterocyclyl group within Q¹ optionally bears an oxo substituent.

26. A quinazoline derivative of the Formula I as defined in any one of the preceding claims, wherein Q¹ is selected from azetidin-3-yl, pyrrolidin-2-yl, pyrrolidin-3-yl, piperidin-3-yl or piperidin-4-yl,
and wherein Q¹ optionally bears one or more substituents, which may be the same or different, selected from fluoro, chloro, hydroxy, carbamoyl, (1-4C)alkyl, (1-4C)alkoxy, N-(1-4C)alkylcarbamoyl and N,N-di-[(1-4C)alkyl]carbamoyl,
and wherein any heterocyclyl group within Q¹ optionally bears an oxo substituent.

27. A quinazoline derivative of the Formula I as defined in any one of claims 1 to 26, wherein M is CO.

28. A quinazoline derivative of the Formula I as defined in any one of claims 1 to 26, wherein M is SO₂.

29. A quinazoline derivative of the Formula I as defined in any one of the preceding claims, wherein X³ is a group of the formula —(CR⁸R⁹)_{q}—, q is 1, 2, 3 or 4, each of R⁸ and R⁹, which may be the same or different, is selected from hydrogen and (1-6C)alkyl,
and wherein any CH₂ or CH₃ group within an X³ group, optionally bears on each said CH₂ or CH₃ group one or more halogeno substituents,
and wherein any CH₂ group which is attached to 2 carbon atoms or any CH₃ group which is attached to a carbon atom within a X³ substituent optionally bears on each said CH₂ or CH₃ group a substituent selected from hydroxy and (1-6C)alkoxy.

30. A quinazoline derivative of the Formula I as defined in any one of the preceding claims, wherein X³ is selected from a group of the formula —(CR⁸R⁹)—, —(CR⁸R⁹CH₂)—, —(CR⁸R⁹CH₂CH₂)—, —(CH₂CR⁸R⁹)— and —(CH₂CH₂CR⁸R⁹)—,
each of R⁸ and R⁹, which may be the same or different, is selected from hydrogen and (1-6C)alkyl, provided that at least one of R⁸ or R⁹ group in X³ is (1-6C)alkyl,
and wherein any CH₂ or CH₃ group within an X³ group, optionally bears on each said CH₂ or CH₃ group one or more halogeno substituents,
and wherein any CH₂ group which is attached to 2 carbon atoms or any CH₃ group which is attached to a carbon atom within a X³ substituent optionally bears on each said CH₂ or CH₃ group a substituent selected from hydroxy and (1-6C)alkoxy.

31. A quinazoline derivative of the Formula I as defined in any one of the preceding claims, wherein X³ is selected from a group of the formula —CH₂)_{q}—, wherein q is 1, 2 or 3.

32. A quinazoline derivative of the Formula I as defined in any one of the preceding claims, wherein X³ is —CH₂—.

33. A quinazoline derivative of the Formula I as defined in any one of the preceding claims, wherein Z is selected from hydrogen, hydroxy, amino, (1-6C)alkylamino, di-[(1-6C)alkyl]amino, (1-6C)alkoxy, and a group of the formula:
Q⁴-X⁵—
wherein X⁵ is a direct bond or is selected from O, N(R¹²), SO₂ and SO₂N(R¹²), wherein R¹² is hydrogen or (1-6C)alkyl, and Q⁴ is (3-7C)cycloalkyl, (3-7C)cycloalkyl-(1-4C)alkyl, (3-7C)cycloalkenyl, (3-7C)cycloalkenyl-(1-4C)alkyl, heterocyclyl or heterocyclyl-(1-4C)alkyl,
provided that when X⁵ is a direct bond, Q⁴ is heterocyclyl,
and provided that when m, p and q are all 0, then Z is heterocyclyl,
and wherein adjacent carbon atoms in any (2-6C)alkylene chain within a Z substituent are optionally separated by the insertion into the chain of a group selected from O, S, SO, SO₂, N(R¹³), CO, —C═C— and —C≡C— wherein R¹³ is hydrogen or (1-6C)alkyl,
and wherein any CH₂ or CH₃ group within a Z group, other than a CH₂ group within a heterocyclyl ring, optionally bears on each said CH₂ or CH₃ group one or more halogeno or (1-6C)alkyl substituents or a substituent selected from hydroxy, cyano, amino, carboxy, carbamoyl, sulfamoyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
and wherein any heterocyclyl group within a Z substituent optionally bears one or more substitutents which may be the same or different, selected from halogeno, trifluoromethyl, cyano, nitro, hydroxy, amino, formyl, mercapto, (1-6C)alkyl, (2-6C)alkenyl, (2-6C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (2-6C)alkanoyl, (2-6C)alkanoyloxy and from a group of the formula:
—X⁶—R¹⁴
wherein X⁶ is a direct bond or is selected from O, CO, SO₂ and N(R¹⁵), wherein R¹⁵ is hydrogen or (1-4C)alkyl, and R¹⁴ is halogeno-(1-4C)alkyl, hydroxy-(1-4C)alkyl, (1-4C)alkoxy-(1-4C)alkyl, cyano-(1-4C)alkyl, amino-(1-4C)alkyl, N-(1-4C)alkylamino-(1-4C)alkyl and N,N-di-[(1-4C)alkyl]amino-(1-4C)alkyl,
and wherein any heterocyclyl group within a Z substituent optionally bears 1 or 2 oxo r thioxo substituents.

34. A quinazoline derivative of the Formula I as defined in any one of the preceding claims, wherein Z is selected from hydrogen, hydroxy, amino, (1-6C)alkylamino, hydroxy-(2-6C)alkylamino, (1-4C)alkoxy-(2-6C)alkylamino, di-[(1-6C)alkyl]amino, N-[hydroxy-(2-6C)alkyl]-N-(1-6C)alkylamino, N-[(1-4C)alkoxy-(2-6C)alkyl]-N-(1-6C)alkylamino, di-[hydroxy-(2-6C)alkyl]-amino, di-[(1-4C)alkoxy-(2-6C)alkyl]amino, N-[(1-4C)alkoxy-(2-6C)alkyl]-N-[hydroxy-(2-6C)alkyl]-amino, (1-6C)alkoxy, hydroxy-(2-6C)alkoxy and (1-4C)alkoxy-(2-6C)alkoxy.

35. A quinazoline derivative of the Formula I as defined in any one of the preceding claims, wherein Z is selected from hydrogen, hydroxy, methoxy, ethoxy, 2-hydroxyethoxy, 2-methoxyethoxy, amino, methylamino, ethylamino, N-(2-hydroxyethyl)amino, N-(2-methoxyethyl)amino, dimethylamino, N-methyl-N-ethylamino, di-ethylamino, N-(2-hydroxyethyl)-N-methylamino, N-(2-hydroxyethyl)-N-ethylamino, N,N-di-(2-hydroxyethyl)amino, N-(2-methoxyethyl)-N-methylamino, N-(2-methoxyethyl)-N-ethylamino, pyrrolidin-1-yl, piperidino, piperazin-1-yl, morpholino, tetrahydrofuranyl and tetrahydropyranyl,
and wherein any heterocyclyl group within Z optionally bears 1 or 2 substituents, which may be the same or different, selected from fluoro, chloro, hydroxy, (1-4C)alkyl and (1-4C)alkoxy.

36. A quinazoline derivative of the Formula I as defined in any one of the preceding claims, Z is selected from hydrogen, hydroxy and dimethylamino.

37. A quinazoline derivative selected from one or more of the following:
2-{4-[(4-{[3-chloro-4-(pyridin-2-ylmethoxy)phenyl]amino}quinazolin-6-yl)oxy]piperidin-1-yl}-2-oxoethanol;
2-((2S)-2-{[(4-{[3-chloro-4-(pyridin-2-ylmethoxy)phenyl]amino}quinazolin-6-yl)oxy]methyl}pyrrolidin-1-yl)-2-oxoethanol;
N-[3-chloro-4-(pyridin-2-ylmethoxy)phenyl]-6-({(2S)-1-[(dimethylamino)acetyl]pyrrolidin-2-yl}methoxy)quinazolin-4-amine;
N-[3-chloro-4-(pyridin-2-ylmethoxy)phenyl]-6-({(3S)-1-[(dimethylamino)acetyl]piperidin-3-yl}oxy)quinazolin-4-amine;
2-{(3S)-3-[(4-{[3-chloro-4-(pyridin-2-ylmethoxy)phenyl]amino}quinazolin-6-yl)oxy]pyrrolidin-1-yl}-2-oxoethanol;
2-{(3S)-3-[(4-{[3-chloro-4-(pyridin-2-ylmethoxy)phenyl]amino}quinazolin-6-yl)oxy]piperidin-1-yl}-2-oxoethanol;
N-{3-chloro-4-[(3-fluorobenzyl)oxy]phenyl}-6-({1-[(dimethylamino)acetyl]piperidin-4-yl}oxy)quinazolin-4-amine;
N-[3-chloro-4-(pyridin-2-ylmethoxy)phenyl]-6-({1-[(dimethylamino)acetyl]piperidin-4-yl}oxy)quinazolin-4-amine;
N-[3-chloro-4-(pyrazin-2-ylmethoxy)phenyl]-6-({1-[(dimethylamino)acetyl]piperidin-4-yl}oxy)quinazolin-4-amine;
6-({1-[(dimethylamino)acetyl]piperidin-4-yl}oxy)-N-{4-[(3-fluorobenzyl)oxy]-3-methoxyphenyl}quinazolin-4-amine;
6-({1-[(dimethylamino)acetyl]piperidin-4-yl}oxy)-N-[4-(pyridin-2-ylmethoxy) phenyl]quinazolin-4-amine;
6-({1-[(dimethylamino)acetyl]piperidin-4-yl}oxy)-N-[3-methoxy-4-(pyridin-2-ylmethoxy)phenyl]quinazolin-4-amine;
6-({1-[(dimethylamino)acetyl]piperidin-4-yl}oxy)-N-{4-[(3-fluorobenzyl)oxy]phenyl}-7-methoxyquinazolin-4-amine;
6-({1-[(dimethylamino)acetyl]piperidin-4-yl}oxy)-N-{4-[(3-fluorobenzyl)oxy]-3-methoxyphenyl}-7-methoxyquinazolin-4-amine;
N-{3-chloro-4-[(3-fluorobenzyl)oxy]phenyl}-6-({1-[(dimethylamino)acetyl]piperidin-4-yl}oxy)-7-methoxyquinazolin-4-amine;
6-({1-[(dimethylamino)acetyl]piperidin-4-yl}oxy)-7-methoxy-N-[4-(pyridin-2-ylmethoxy)phenyl]quinazolin-4-amine;
6-({1-[(dimethylamino)acetyl]piperidin-4-yl}oxy)-7-methoxy-N-[3-methoxy-4-(pyridin-2-ylmethoxy)phenyl]quinazolin-4-amine;
N-[3-chloro-4-(pyridin-2-ylmethoxy)phenyl]-6-({1-[(dimethylamino)acetyl]piperidin-4-yl}oxy)-7-methoxyquinazolin-4-amine;
N-[3-chloro-4-(pyrazin-2-ylmethoxy)phenyl]-6-({1-[(dimethylamino)acetyl]piperidin-4-yl}oxy)-7-methoxyquinazolin-4-amine;
6-({1-[(dimethylamino)acetyl]piperidin-4-yl}oxy)-7-methoxy-N-[4-(pyrazin-2-ylmethoxy)phenyl]quinazolin-4-amine;
6-({1-[(dimethylamino)acetyl]piperidin-4-yl}oxy)-7-methoxy-N-[3-methoxy-4-(pyrazin-2-ylmethoxy)phenyl]quinazolin-4-amine;
6-({1-[(dimethylamino)acetyl]piperidin-4-yl}oxy)-N-[4-(3-fluorobenzyloxy)phenyl]quinazolin-4-amine;
6-({1-[(dimethylamino)acetyl]piperidin-4-yl}oxy)-N-[3-methoxy-4-(pyrazin-2-ylmethoxy)phenyl]quinazolin-4-amine;
6-({1-[(dimethylamino)acetyl]piperidin-4-yl}oxy)-N-[4-(pyrazin-2-ylmethoxy)phenyl]quinazolin-4-amine;
N-[3-chloro-4-(pyridin-2-ylmethoxy)phenyl]-6-{[1-(methylsulfonyl)pyrrolidin-3-yl]methoxy}quinazolin-4-amine;
2-{4-[(4-{[3-chloro-4-(pyridin-2-ylmethoxy)phenyl]amino}-7-methoxyquinazolin-6-yl)oxy]piperidin-1-yl}-2-oxoethanol;
6-[(1-acetylpiperidin-4-yl)oxy]-N-[3-chloro-4-(pyridin-2-ylmethoxy)phenyl]-7-methoxyquinazolin-4-amine;
2-{4-[(4-{[3-chloro-4-(pyrazin-2-ylmethoxy)phenyl]amino}-7-methoxyquinazolin-6-yl)oxy]piperidin-1-yl}-2-oxoethanol;
6-[(1-acetylpiperidin-4-yl)oxy]-N-[3-chloro-4-(pyrazin-2-ylmethoxy)phenyl]-7-methoxyquinazolin-4-amine;
6-[(1-acetylpiperidin-4-yl)oxy]-N-[3-chloro-4-(pyridin-2-ylmethoxy)phenyl]quinazolin-4-amine;
2-{4-[(4-{[3-chloro-4-(pyrazin-2-ylmethoxy)phenyl]amino}quinazolin-6-yl)oxy]piperidin-1-yl}-2-oxoethanol;
6-[(1-acetylpiperidin-4-yl)oxy]-N-[3-chloro-4-(pyrazin-2-ylmethoxy)phenyl]quinazolin-4-amine;
N-[3-chloro-4-(pyridin-2-ylmethoxy)phenyl]-6-{[1-(methylsulfonyl)piperidin-4-yl]oxy}quinazolin-4-amine;
N-{3-ethynyl-4-[(3-fluorobenzyl)oxy]phenyl}-7-methoxy-6-{[1-(methylsulfonyl)piperidin-4-yl]oxy}quinazolin-4-amine;
7-methoxy-6-{[1-(methylsulfonyl)piperidin-4-yl]oxy}-N-[4-(1,3-thiazol-2-ylthio)phenyl]quinazolin-4-amine;
N-[3-chloro-4-(pyrazin-2-ylmethoxy)phenyl]-6-{[1-(methylsulfonyl)piperidin-4-yl]oxy}quinazolin-4-amine;
N-{3-fluoro-4-[(1-methyl-1H-imidazol-2-yl)thio]phenyl}-6-{[1-(methylsulfonyl)piperidin-4-yl]oxy}quinazolin-4-amine;
N-{3-chloro-4-[(1-methyl-1H-imidazol-2-yl)thio]phenyl}-6-{[1-(methylsulfonyl)piperidin-4-yl]oxy}quinazolin-4-amine;
6-{[1-(methylsulfonyl)piperidin-4-yl]oxy}-N-[4-(1,3-thiazol-2-ylthio)phenyl]quinazolin-4-amine;
N-{3-fluoro-4-[(1-methyl-1H-imidazol-2-yl)thio]phenyl}-7-methoxy-6-{[1-(methylsulfonyl)piperidin-4-yl]oxy}quinazolin-4-amine;
2-(4-{[4-({3-methyl-4-[(6-methylpyridin-3-yl)oxy]phenyl}amino)quinazolin-6-yl]oxy}piperidin-1-yl)-2-oxoethanol;
2-{3-[(4-{[3-chloro-4-(pyridin-2-ylmethoxy)phenyl]amino}quinazolin-6-yl)oxy]azetidin-1-yl}-2-oxoethanol; and
2-(3-{[4-({3-methyl-4-[(6-methylpyridin-3-yl)oxy]phenyl}amino)quinazolin-6-yl]oxy}azetidin-1-yl)-2-oxoethanol;
or a pharmaceutically acceptable salt thereof.

38. A pharmaceutical composition which comprises a quinazoline derivative of the Formula I, or a pharmaceutically acceptable salt thereof, as defined in any one of claims 1 to 37 in association with a pharmaceutically-acceptable diluent or carrier.

39. A quinazoline derivative of the Formula I, or a pharmaceutically acceptable salt thereof, as defined in any one of claims 1 to 37 for use as a medicament.

40. A quinazoline derivative of the Formula I, or a pharmaceutically acceptable salt thereof, as defined in any one of claims 1 to 37 for use in the production of an anti-proliferative effect which effect is produced alone or in part by inhibiting erbB2 receptor tyrosine kinase in a warm-blooded animal such as man.

41. A quinazoline derivative of the Formula I, or a pharmaceutically acceptable salt thereof, as defined in any one of claims 1 to 37 for use in the production of an erbB2 receptor tyrosine kinase inhibitory effect in a warm-blooded animal such as man.

42. A quinazoline derivative of the Formula I, or a pharmaceutically acceptable salt thereof, as defined in any one of claims 1 to 37 for use in the production of a selective erbB2 receptor tyrosine kinase inhibitory effect in a warm-blooded animal such as man.

43. A process for the preparation of a quinazoline derivative of the Formula I, or a pharmaceutically acceptable salt thereof, as defined in claim 1 which comprises:
Process (a) for the preparation of compounds of the Formula I wherein M is CO, the coupling, conveniently in the presence of a suitable base, of a quinazoline of the formula II: 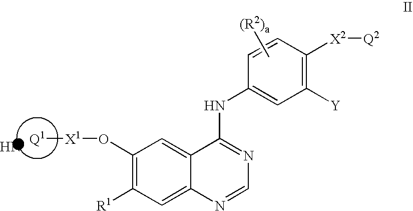
wherein R¹, R², X¹, X² Y, a, Q¹ and Q² have any of the meanings defined in claim 1 except that any functional group is protected if necessary, with a carboxylic acid of the formula III, or a reactive derivative thereof:
Z-X³—COOH III
wherein Z and X³ have any of the meanings defined in claim 1 except that any functional group is protected if necessary;
or
Process (b) the reaction, conveniently in the presence of a suitable base, of a quinazoline of the formula II as hereinbefore defined in relation to Process (a), with a compound of the formula IV:
Z-X³-M-L¹ IV
wherein L¹ is a displaceable group and Z, X³ and M have any of the meanings defined in claim 1 except that any functional group is protected if necessary; or
Process (c) for the preparation of those compounds of the Formula I wherein Z is linked to X³ by nitrogen, the reaction, conveniently in the presence of a suitable base, of a compound of the formula V: 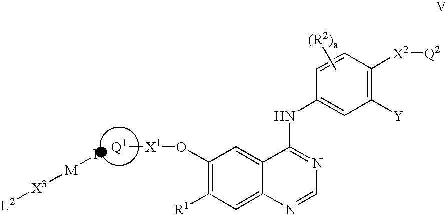
wherein L² is a displaceable group and R¹, R², X¹, X², X³, Y, M, a, Q¹ and Q² have any of the meanings defined in claim 1 except that any functional group is protected if necessary, with a compound of the formula ZH, wherein Z is as defined in claim 1, except that any functional group is protected if necessary; or
Process (d) the reaction, conveniently in the presence of a suitable base, of a quinazoline of the formula VI: 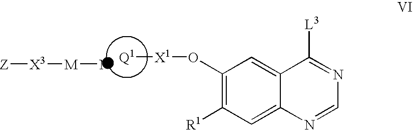
wherein, L³ is a displaceable group and R¹, X¹, X³, Z, and Q¹ have any of the meanings defined in claim 1 except that any functional group is protected if necessary, with a compound of the formula VII: 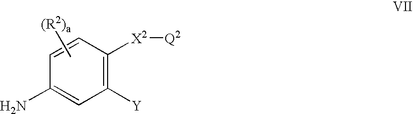
wherein R², a, X², Q² and Y have any of the meanings defined in claim 1 except that any functional group is protected if necessary; or
Process (e) for the preparation of those compounds of the Formula I wherein X² is OC(R⁴)₂, SC(R⁴)₂ or N(R⁴)C(R⁴)₂, the reaction, conveniently in the presence of a suitable base, of a quinazoline of the formula VIII: 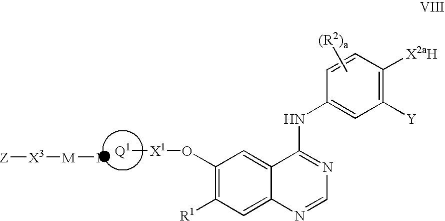
wherein X^{2a} is O, S or N(R⁴) and R¹, R², X¹, X², X³, M, Z, Y, a and Q¹ have any of the meanings defined in claim 1 except that any functional group is protected if necessary, with a compound of the formula IX:
Q²C(R⁴)₂-L⁴ IX
wherein L⁴ is a suitable displaceable group and Q² and R⁴ have any of the meanings defined in claim 1 except that any functional group is protected if necessary; or
Process (f) for the preparation of those compounds of the Formula I wherein X² is OC(R⁴)₂, the coupling of a quinazoline of the formula X: 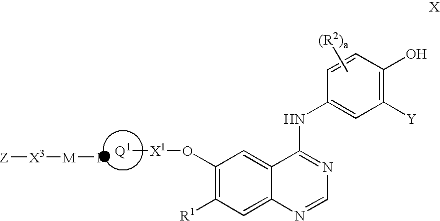
wherein R¹, R², X¹, X², X³, M, Z, Y, a and Q¹ have any of the meanings defined in claim 1 except that any functional group is protected if necessary, with an alcohol of the formula XI:
Q²-C(R⁴)₂—OH XI
wherein Q² and R⁴ have any of the meanings defined in claim 1 except that any functional group is protected if necessary; or
Process (g) the coupling of a quinazoline compound of the formula XII: 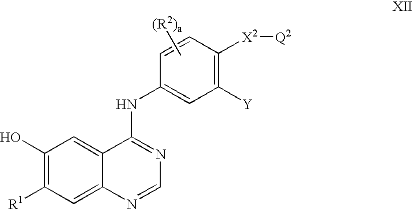
wherein R¹, R², X², a and Y have any of the meanings in claim 1 except that any functional group is protected if necessary, with an alcohol of the formula XIII: 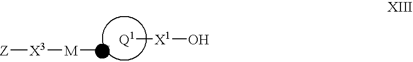
wherein X¹, X³, M, Z, and Q¹ have any of the meanings defined in claim 1 except that any functional group is protected if necessary; or
Process (A) the reaction, conveniently in the presence of a suitable base, of a quinazoline of the formula XII, as defined in relation to Process (g) with a compound of the formula MV: 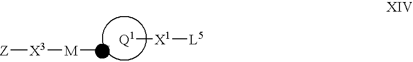
wherein L⁵ is a displaceable group and X¹, X³, M and Z, and Q¹ have any of the meanings defined in claim 1 except that any functional group is protected if necessary;
and thereafter, if necessary:
(i) converting a quinazoline derivative of the formula I into another quinazoline derivative of the formula I;
(ii) removing any protecting group that is present by conventional means;
(iii) forming a pharmaceutically acceptable salt.
